(19) United States
(12) Patent Application Publication
(10) Pub. No.: US 2010/0069399 A1
(43) Pub. Date: Mar. 18, 2010

(54) ARYLPIPERAZINE MODULATORS OF D2 RECEPTORS, 5-HT1A RECEPTORS, AND/OR 5-HT2A RECEPTORS

(75) Inventors: Thomas G. Gant, Carlsbad, CA (US); Sepehr Sarshar, Cardiff by the Sea, CA (US); Chengzhi Zhang, San Diego, CA (US)

Correspondence Address: GLOBAL PATENT GROUP - APX, 10411 Clayton Road, Suite 304, ST. LOUIS, MO, 63131, US

(73) Assignee: AUSPEX PHARMACEUTICAL, INC., Vista, CA (US)

(21) Appl. No.: 12/395,187

(22) Filed: Feb. 27, 2009

Related U.S. Application Data
(60) Provisional application No. 61/096,871, filed on Sep. 15, 2008.

(51) Int. Cl. A61K 31/496; C07D 401/12; A61P 25/18

(52) U.S. Cl. 514/253.07; 544/363

(57) ABSTRACT

The present invention relates to new arylpiperazine modulators of D2 receptors, 5-HT1A receptors, and/or 5-HT2A receptors, pharmaceutical compositions thereof, and methods of use thereof.

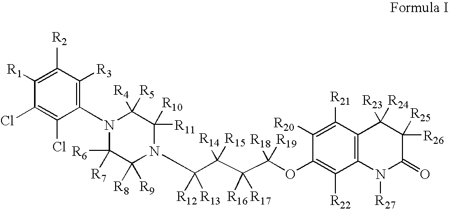

This application claims the benefit of priority of U.S. provisional application No. 61/096,871, filed Sep. 15, 2008, the disclosure of which is hereby incorporated by reference as if written herein in its entirety.

Disclosed herein are new arylpiperazine compounds and compositions and their application as pharmaceuticals for the treatment of disorders. Methods of modulation of D2 receptor, 5-HT1A receptor, and/or 5-HT2A receptor activity in a subject are also provided for the treatment of disorders such as schizophrenia, attention deficit hyperactivity disorder, autism, drug dependence, acute bipolar mania, bipolar disorder, and major depressive disorder.

Aripiprazole (ABILIFY®, ABILITAT®, OPC 14597, OPC-31, CAS #129722-12-9), 7-[4-[4-(2,3-dichlorophenyl)piperazin-1-yl]butoxy]-3,4-dihydro-1H-quinolin-2-one, is a D2 receptor partial agonist, 5-HT1A receptor partial agonist, and 5-HT2A receptor antagonist. Aripiprazole is commonly prescribed for the treatment of bipolar disorder, major depressive disorder, and schizophrenia. Drug Report for Aripiprazole, Thompson Investigational Drug Database, (Aug. 12, 2008); Fleischhacker, Expert Opin. Pharmacothe., 2005, 6(12), 2091-2101; and Sanford et al., CNS Drugs, 2008, 22(4), 335-52. Aripiprazole has also shown promise in treating attention deficit hyperactivity disorder, autism, and drug dependence. Drug Report for Aripiprazole, Thompson Investigational Drug Database, (Aug. 12, 2008).

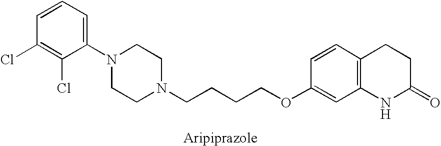

The aripiprazole chemical structure contains a number of features that we posit will produce inactive or toxic metabolites, the formation of which can be reduced by the approach described herein. Aripiprazole is subject to metabolic oxidation (by CYP3A4 and CYP2D6) at the dihydroquinolone ring to give dehydroaripiprazole, an active metabolite. Fleischhacker, Expert Opin. Pharmacother., 2005, 6(12), 2091-2101. Both aripiprazole and dehydroaripiprazole are subject to further oxidation by CYP3A4 and CYP2D6, likely resulting in N-dealkylated metabolites. Kubo et al., Drug Metab. Pharmacokinet., 2007, 22(5), 358-66. The conversion of aripiprazole to dehydroaripiprazole, as well as other metabolic transformations, occur in part through polymorphically-expressed enzymes such as CYP2D6, exacerbating interpatient variability. Kubo et al., Drug Metab. Pharmacokinet., 2007, 22(5), 358-66; Hendset et al., Eur. J. Clin. Pharmacol., 2007, 63, 1147-51. Additionally, some aripiprazole metabolites may have undesirable side effects. Adverse effects associated with the administration of aripiprazole include extrapyramidal disorder, insomnia, akathisia, anxiety, constipation, dizziness, drowsiness, dyspepsia, headache, and weight loss. In order to overcome its short half-life, the drug must be taken at least once daily, which increases the probability of patient incompliance and discontinuance. Further, abruptly stopping treatment with aripiprazole can lead to withdrawal or discontinuation syndrome. Medicines with longer half-lives will likely attenuate these deleterious effects.

### Deuterium Kinetic Isotope Effect

In order to eliminate foreign substances such as therapeutic agents, the animal body expresses various enzymes, such as the cytochrome P₄₅₀ enzymes (CYPs), esterases, proteases, reductases, dehydrogenases, and monoamine oxidases, to react with and convert these foreign substances to more polar intermediates or metabolites for renal excretion. Such metabolic reactions frequently involve the oxidation of a carbon-hydrogen (C—H) bond to either a carbon-oxygen (C—O) or a carbon-carbon (C—C) π-bond. The resultant metabolites may be stable or unstable under physiological conditions, and can have substantially different pharmacokinetic, pharmacodynamic, and acute and long-term toxicity profiles relative to the parent compounds. For most drugs, such oxidations are generally rapid and ultimately lead to administration of multiple or high daily doses.

The relationship between the activation energy and the rate of reaction may be quantified by the Arrhenius equation, k=Ae^{-Eact/RT}. The Arrhenius equation states that, at a given temperature, the rate of a chemical reaction depends exponentially on the activation energy (E_{act}).

The transition state in a reaction is a short lived state along the reaction pathway during which the original bonds have stretched to their limit. By definition, the activation energy E_{act} for a reaction is the energy required to reach the transition state of that reaction. Once the transition state is reached, the molecules can either revert to the original reactants, or form new bonds giving rise to reaction products. A catalyst facilitates a reaction process by lowering the activation energy leading to a transition state. Enzymes are examples of biological catalysts.

Carbon-hydrogen bond strength is directly proportional to the absolute value of the ground-state vibrational energy of the bond. This vibrational energy depends on the mass of the atoms that form the bond, and increases as the mass of one or both of the atoms making the bond increases. Since deuterium (D) has twice the mass of protium (¹H), a C-D bond is stronger than the corresponding C—¹H bond. If a C—¹H bond is broken during a rate-determining step in a chemical reaction (i.e. the step with the highest transition state energy), then substituting a deuterium for that protium will cause a decrease in the reaction rate. This phenomenon is known as the Deuterium Kinetic Isotope Effect (DKIE). The magnitude of the DKIE can be expressed as the ratio between the rates of a given reaction in which a C—¹H bond is broken, and the same reaction where deuterium is substituted for protium. The DKIE can range from about 1 (no isotope effect) to very large numbers, such as 50 or more. Substitution of tritium for hydrogen results in yet a stronger bond than deuterium and gives numerically larger isotope effects

Deuterium (²H or D) is a stable and non-radioactive isotope of hydrogen which has approximately twice the mass of protium (¹H), the most common isotope of hydrogen. Deuterium oxide (D₂O or “heavy water”) looks and tastes like H₂O, but has different physical properties.

When pure D₂O is given to rodents, it is readily absorbed. The quantity of deuterium required to induce toxicity is extremely high. When about 0-15% of the body water has been replaced by D₂O, animals are healthy but are unable to gain weight as fast as the control (untreated) group. When about 15-20% of the body water has been replaced with D₂O, the animals become excitable. When about 20-25% of the body water has been replaced with D₂O, the animals become so excitable that they go into frequent convulsions when stimulated. Skin lesions, ulcers on the paws and muzzles, and necrosis of the tails appear. The animals also become very aggressive. When about 30% of the body water has been replaced with D₂O, the animals refuse to eat and become comatose. Their body weight drops sharply and their metabolic rates drop far below normal, with death occurring at about 30 to about 35% replacement with D₂O. The effects are reversible unless more than thirty percent of the previous body weight has been lost due to D₂O. Studies have also shown that the use of D₂O can delay the growth of cancer cells and enhance the cytotoxicity of certain antineoplastic agents.

Deuteration of pharmaceuticals to improve pharmacokinetics (PK), pharmacodynamics (PD), and toxicity profiles has been demonstrated previously with some classes of drugs. For example, the DKIE was used to decrease the hepatotoxicity of halothane, presumably by limiting the production of reactive species such as trifluoroacetyl chloride. However, this method may not be applicable to all drug classes. For example, deuterium incorporation can lead to metabolic switching. Metabolic switching occurs when xenogens, sequestered by Phase I enzymes, bind transiently and re-bind in a variety of conformations prior to the chemical reaction (e.g., oxidation). Metabolic switching is enabled by the relatively vast size of binding pockets in many Phase I enzymes and the promiscuous nature of many metabolic reactions. Metabolic switching can lead to different proportions of known metabolites as well as altogether new metabolites. This new metabolic profile may impart more or less toxicity. Such pitfalls are non-obvious and are not predictable a priori for any drug class.

Aripiprazole is a D2 receptor partial agonist, 5-HT1A receptor partial agonist, and 5-HT2A receptor antagonist. The carbon-hydrogen bonds of aripiprazole contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms). Increased levels of deuterium incorporation may produce a detectable Deuterium Kinetic Isotope Effect (DKIE) that could effect the pharmacokinetic, pharmacologic and/or toxicologic profiles of such aripiprazole in comparison with the compound having naturally occurring levels of deuterium.

Based on discoveries made in our laboratory, as well as considering the literature, aripiprazole is metabolized in humans at the dihydroquinolone ring and the methylene group alpha to the piperazine group. The current approach has the potential to prevent metabolism at these sites. Other sites on the molecule may also undergo transformations leading to metabolites with as-yet-unknown pharmacology/toxicology. Limiting the production of these metabolites has the potential to decrease the danger of the administration of such drugs and may even allow increased dosage and/or increased efficacy. All of these transformations can occur through polymorphically-expressed enzymes, exacerbating interpatient variability. Further, some disorders are best treated when the subject is medicated around the clock or for an extended period of time. For all of the foregoing reasons, a medicine with a longer half-life may result in greater efficacy and cost savings. Various deuteration patterns can be used to (a) reduce or eliminate unwanted metabolites, (b) increase the half-life of the parent drug, (c) decrease the number of doses needed to achieve a desired effect, (d) decrease the amount of a dose needed to achieve a desired effect, (e) increase the formation of active metabolites, if any are formed, (f) decrease the production of deleterious metabolites in specific tissues, and/or (g) create a more effective drug and/or a safer drug for polypharmacy, whether the polypharmacy be intentional or not. The deuteration approach has the strong potential to slow the metabolism of aripiprazole and attenuate interpatient variability.

Novel compounds and pharmaceutical compositions, certain of which have been found to modulate D2 receptors, 5-HT1A receptors, and/or 5-HT2A receptors have been discovered, together with methods of synthesizing and using the compounds, including methods for the treatment of D2 receptor-mediated disorders, 5-HT1A receptor-mediated disorders, and/or 5-HT2A receptor-mediated disorders in a patient by administering the compounds.

In certain embodiments of the present invention, compounds have structural Formula I:

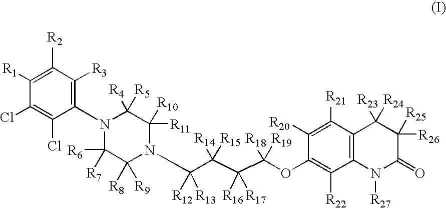

or a salt, solvate, or prodrug thereof, wherein:

R₁-R₂₇ are independently selected from the group consisting of hydrogen and deuterium; and

at least one of R₁-R₂₇ is deuterium.

Certain compounds disclosed herein may possess useful D2 receptor, 5-HT1A receptor, and/or 5-HT2A receptor modulating activity, and may be used in the treatment or prophylaxis of a disorder in which D2 receptors, 5-HT1A receptors, and/or 5-HT2A receptors play an active role. Thus, certain embodiments also provide pharmaceutical compositions comprising one or more compounds disclosed herein together with a pharmaceutically acceptable carrier, as well as methods of making and using the compounds and compositions. Certain embodiments provide methods for modulating D2 receptors, 5-HT1A receptors, and/or 5-HT2A receptors. Other embodiments provide methods for treating a D2 receptor-mediated disorder, a 5-HT1A receptor-mediated disorder, and/or a 5-HT2A receptor-mediated disorder in a patient in need of such treatment, comprising administering to said patient a therapeutically effective amount of a compound or composition according to the present invention. Also provided is the use of certain compounds disclosed herein for use in the manufacture of a medicament for the prevention or treatment of a disorder ameliorated by the modulation of D2 receptors, 5-HT1A receptors, and/or 5-HT2A receptors.

The compounds as disclosed herein may also contain less prevalent isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen.

In certain embodiments, the compound disclosed herein may expose a patient to a maximum of about 0.000005% D₂O or about 0.00001% DHO, assuming that all of the C-D bonds in the compound as disclosed herein are metabolized and released as D₂O or DHO. In certain embodiments, the levels of D₂O shown to cause toxicity in animals is much greater than even the maximum limit of exposure caused by administration of the deuterium enriched compound as disclosed herein. Thus, in certain embodiments, the deuterium-enriched compound disclosed herein should not cause any additional toxicity due to the formation of D₂O or DHO upon drug metabolism.

In certain embodiments, the deuterated compounds disclosed herein maintain the beneficial aspects of the corresponding non-isotopically enriched molecules while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions.

In certain embodiments, if R₁₂-R₁₃ and R₂₃-R₂₆ are deuterium, then at least one of R₁-R₁₁, R₁₄-R₂₂ and R₂₇ is deuterium.

In other embodiments, if R₂₃-R₂₆ are deuterium, then at least one of R₁-R₂₂ and R₂₇ is deuterium.

In other embodiments, if R₂₃-R₂₄ are deuterium, then at least one of R₁-R₂₂ and R₂₅-R₂₇ is deuterium.

In other embodiments, if R₁₂-R₁₃ and R₂₃-R₂₄ are deuterium, then at least one of R₁-R₁₁, R₁₄-R₂₂ and R₂₅-R₂₇ is deuterium.

In certain embodiments of the present invention, compounds have structural Formula II:

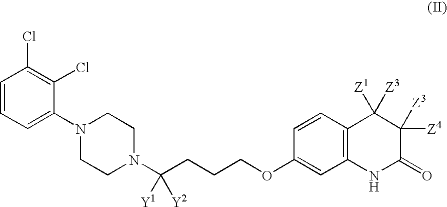

or a pharmaceutically acceptable salt, solvate, or hydrate thereof, wherein:

each Y is independently selected from hydrogen and deuterium; and

each Z is independently selected from hydrogen and deuterium, wherein:

at least one Y or Z is deuterium.

In further embodiments, each of Y¹ and Y² is the same.

In further embodiments, Y¹ and Y² are simultaneously deuterium.

In further embodiments, each of Z¹ and Z² is the same.

In yet further embodiments, Z¹ and Z² are simultaneously deuterium.

In certain embodiments, each of Z³ and Z⁴ is the same.

In certain embodiments, Z³ and Z⁴ are simultaneously deuterium.

In further embodiments, at least two of Y¹, Y², Z¹, Z², Z³, and Z⁴ are deuterium.

In further embodiments, at least three of Y¹, Y², Z¹, Z², Z³, and Z⁴ are deuterium.

In certain embodiments of the present invention, compounds have structural Formula III:

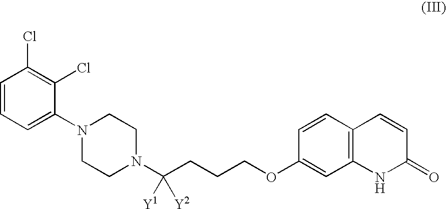

or a pharmaceutically acceptable salt, solvate, or hydrate thereof, wherein:

Y³ is deuterium; and

Y⁴ is selected from hydrogen or deuterium.

In further embodiments, Y⁴ is deuterium.

In yet further embodiments, a compound of this invention is selected from the group consisting of:

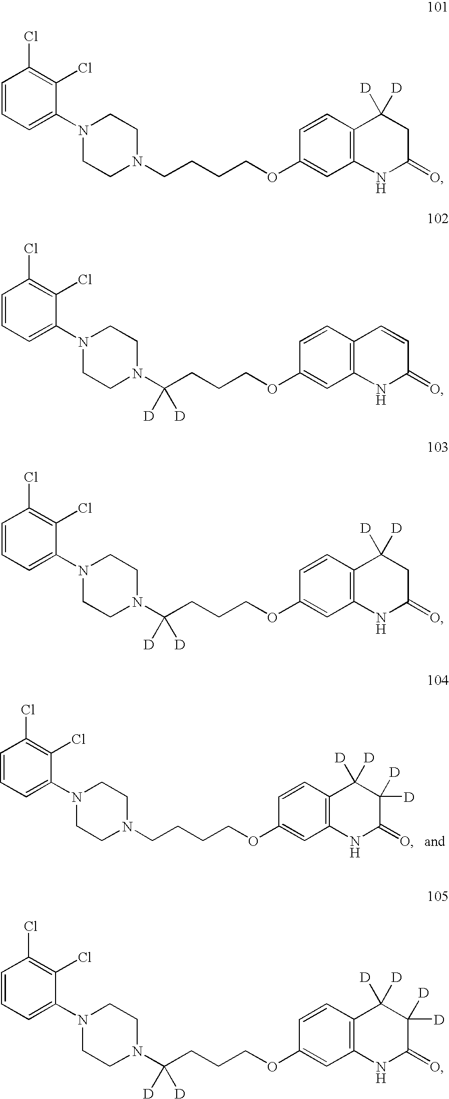

or a HCl salt thereof.

In certain embodiments, each atom not designated as deuterium is present at its naturally abundant isotopic state.

In certain embodiments, a composition comprises:
a. an effective amount of a compound as described herein; and
b. an acceptable carrier.

In further embodiments, the composition as described herein is formulated for pharmaceutical use; and the carrier is a pharmaceutically acceptable carrier.

In further embodiments, the composition as described herein is formulated for oral administration.

In yet further embodiments, the composition as described herein is in the form of a pill, capsule or tablet.

In yet further embodiments, the composition as described herein is in dosage unit form, comprising from 0.1 to 250 mg of a compound as described herein.

In other embodiments, the composition as described herein comprises from 2 to 50 mg of a compound as described herein.

In certain embodiments, the composition described herein further comprises an effective amount of a second therapeutic agent, wherein said second therapeutic agent is useful for treating or preventing a disease or a condition selected from schizophrenia, depression, bipolar depression, depressive disorder, refractive bipolar disorder, autism, alcoholism, cocaine dependency, attention deficit hyperactivity disorder, mood disorders, post traumatic stress disorder, premenstrual dysphoric disorder, nausea, psychotic disorder, tardive dyskinesia, epilepsy, compulsivity, impulsivity, cognition enhancement, weight management, sexual disorders including Hypoactive Sexual Desire Disorder, loss of sexual desire, lack of sexual desire, decreased sexual desire, inhibited sexual desire, loss of libido, libido disturbance, and frigidity.

In certain embodiments, said second therapeutic agent is selected from a NK3 receptor antagonist; a Gly Transporter Type I inhibitor; memantine; an AMPA receptor potentiator; a GABA modulator, anticonvulsant, or benzodiazepine; an antidepressant; a nicotinic receptor agonist or antagonist; a serotonin reuptake inhibitor; sabcomeline, a M1/M4 receptor agonist; an opioid antagonist; D-cycloserine; lamotrigine; methylphenidate; divalproex; clozapine; H1-receptor; an adenosine A2a receptor antagonist; COX-2 inhibitor; an azabicyclo compound for treating CNS disorders; flibanserin; lithium; a pharmaceutically acceptable salt of any of the foregoing additional therapeutic agents; and combinations of the foregoing therapeutic agents.

In certain embodiments, a compound as described herein is a substantially isolated isomer.

In certain embodiments, an article of manufacture comprises separate dosage forms of:
a. a compound as described herein; and
b. a second therapeutic agent, wherein both dosage forms are in a single container.

In certain embodiments, a method for treating a patient suffering from or susceptible to a disorder beneficially treated by an atypical antipsychotic agent, comprises the step of administering to the patient in need thereof a composition as described herein.

In further embodiments, the disorder is selected from schizophrenia, mania or bipolar disorder.

In further embodiments, the disorder is selected from major depressive disorder, ADHD, autism, conduct disorder, anxiety disorder, social anxiety disorder, substance abuse, prodromal psychosis, Tourette's disorder, Asperger's disorder, pervasive developmental disorder, or alcoholism.

In further embodiments, the method as described herein comprises the additional step of coadministering to the patient in need thereof a second therapeutic agent selected from a NK3 receptor antagonist; a Gly Transporter Type I inhibitor; memantine; an AMPA receptor potentiator; a GABA modulator, anticonvulsant, or benzodiazepine; an antidepressant; a nicotinic receptor agonist or antagonist; a serotonin reuptake inhibitor; sabcomeline, a M1/M4 receptor agonist; an opioid antagonist; D-cycloserine; lamotrigine; methylphenidate; divalproex; clozapine; H1-receptor; an adenosine A2a receptor antagonist; COX-2 inhibitor; an azabicyclo compound for treating CNS disorders; flibanserin; lithium; a pharmaceutically acceptable salt of any of the foregoing second therapeutic agents; or combinations of the foregoing second therapeutic agents.

In certain embodiments:
a. the patient suffers from or is susceptible to schizophrenia or bipolar disorder; and the second therapeutic agent is selected from clozapine, depakote ER, or lamotrigine;
b. the patient suffers from or is susceptible to ADHD; and the second therapeutic agent is methylphenidate;
c. the patient suffers from or is susceptible to autism; and the second therapeutic agent is D-cycloserine; or
d. the patient suffers from or is susceptible to alcoholism; and the second therapeutic agent is an opioid antagonist.

In certain embodiments, the pharmaceutical composition is for use in the treatment of a condition selected from schizophrenia, bipolar disorder, bipolar mania, autism, alcoholism, agitation, attention deficit/hyperactivity disorder, anxiety, behavioral disorder, dementia, Alzheimer's dementia, Asperger's disorder, conduct disorder, depression, drug dependency, insulin resistance, mania, obsessive-compulsive disorder, Parkinson's disease, psychosis associated with dementia, drug-induced psychosis, pervasive developmental disorder, prodromal schizophrenia, prodromal psychoses, schizoaffective disorder, social anxiety, tic, and Tourette's disorder, said composition comprising a compound as described herein; and a pharmaceutically acceptable carrier.

In further embodiments, the use is the treatment of schizophrenia, mania or bipolar disorder.

In yet further embodiments, a composition as described herein additionally comprises a second therapeutic agent selected from clozapine, depakote ER, and lamotrigine.

In certain embodiments, a deuterium-enriched compound of formula IV or a pharmaceutically acceptable salt thereof:

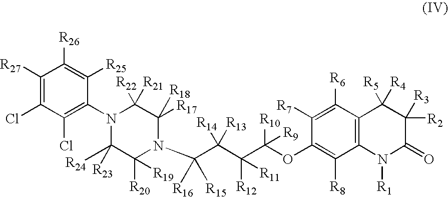

wherein R₁-R₂₇ are independently selected from H and D; and the abundance of deuterium in R₁-R₂₇ is at least 4%.

In further embodiments, a deuterium-enriched compound of structural formula IV, wherein the abundance of deuterium in R₁-R₂₇ is selected from at least 4%, at least 7%, at least 15%, at least 22%, at least 30%, at least 37%, at least 44%, at least 52%, at least 59%, at least 67%, at least 74%, at least 81%, at least 89%, at least 96%, and 100%.

In further embodiments, a deuterium-enriched compound of structural formula IV, wherein the abundance of deuterium in R₁ is 100%.

In further embodiments, a deuterium-enriched compound of structural formula IV, wherein the abundance of deuterium in R₂-R₃ is selected from at least 50% and 100%.

In further embodiments, deuterium-enriched compound of structural formula IV, wherein the abundance of deuterium in R₆-R₈ and R₂₅-R₂₇ is selected from at least 17%, at least 33%, at least 50%, at least 67%, at least 83%, and 100%.

In further embodiments, a deuterium-enriched compound of structural formula IV, wherein the abundance of deuterium in R₁ and R₂-R₃ is selected from at least 33%, at least 67%, and 100%.

In further embodiments, a deuterium-enriched compound of structural formula IV, wherein the abundance of deuterium in R₁, R₆-R₈, and R₂₅-R₂₇ is selected from at least 14%, at least 29%, at least 43%, at least 57%, at least 71%, at least 86%, and 100%.

In further embodiments, a deuterium-enriched compound of structural formula IV, wherein the abundance of deuterium in R₂-R₃, R₆-R₈, and R₂₅-R₂₇ is selected from at least 13%, at least 25%, at least 38%, at least 50%, at least 63%, at least 75%, at least 88%, and 100%.

In further embodiments, a deuterium-enriched compound of structural formula IV, wherein the abundance of deuterium in R₁, R₂-R₃, R₆-R₈, and R₂₅-R₂₇ is selected from at least 11%, at least 22%, at least 33%, at least 44%, at least 56%, at least 67%, at least 78%, and 100%.

In further embodiments, a deuterium-enriched compound of structural formula IV, wherein the abundance of deuterium in R₆-R₂₇ is selected from at least 5%, at least 9%, at least 14%, at least 18%, at least 23%, at least 27%, at least 32%, at least 36%, at least 41%, at least 45%, at least 50%, at least 55%, at least 59%, at least 64%, at least 68%, at least 73%, at least 77%, at least 82%, at least 86%, at least 91%, at least 95%, and 100%.

In further embodiments, a deuterium-enriched compound of structural formula IV, wherein the abundance of deuterium in R₂-R₅ is selected from at least 25%, at least 50%, at least 75%, and 100%.

In yet further embodiments, a deuterium-enriched compound of structural formula IV, wherein the abundance of deuterium in R₉-R₁₆ is selected from at least 13%, at least 25%, at least 38%, at least 50%, at least 63%, at least 75%, at least 88%, and 100%.

In yet even further embodiments, a deuterium-enriched compound of structural formula IV, wherein the abundance of deuterium in R₁₇-R₂₄ is selected from at least 13%, at least 25%, at least 38%, at least 50%, at least 63%, at least 75%, at least 88%, and 100%.

In certain embodiments, a deuterium-enriched compound of structural formula IV, wherein said compound is selected from the group consisting of:

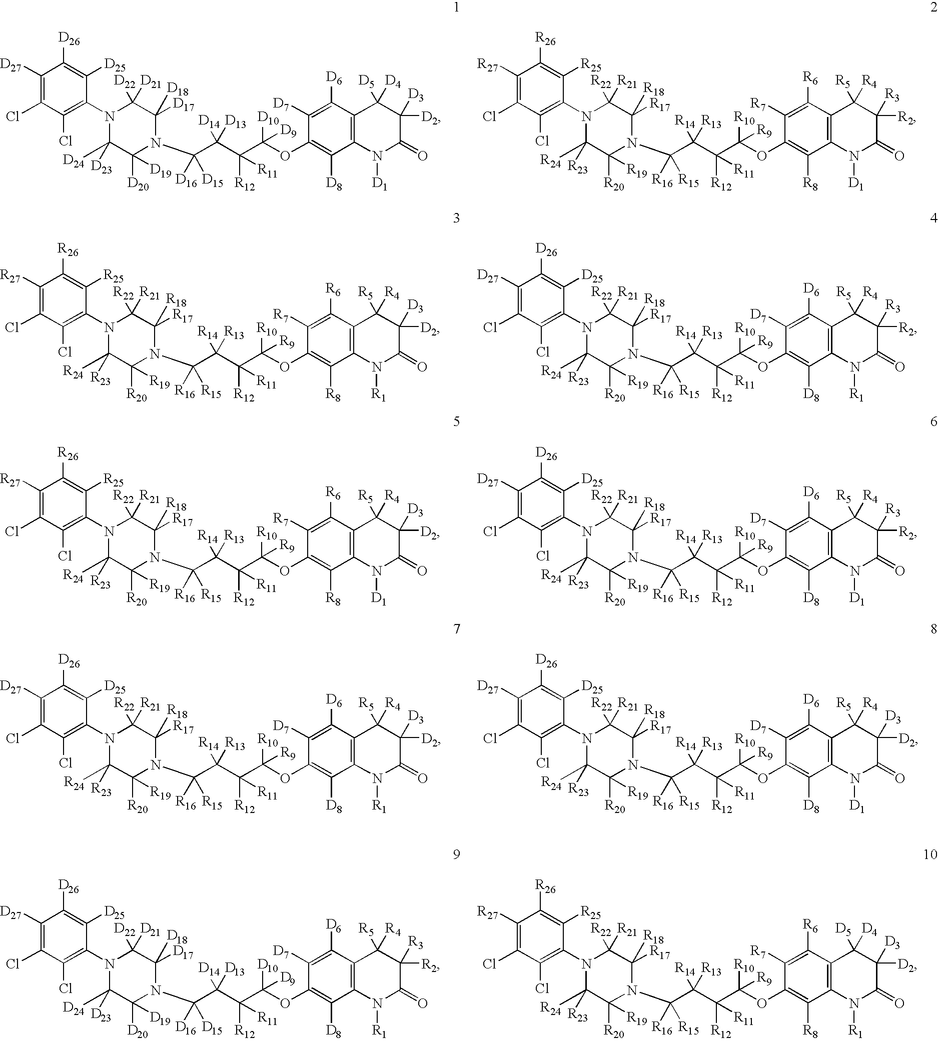

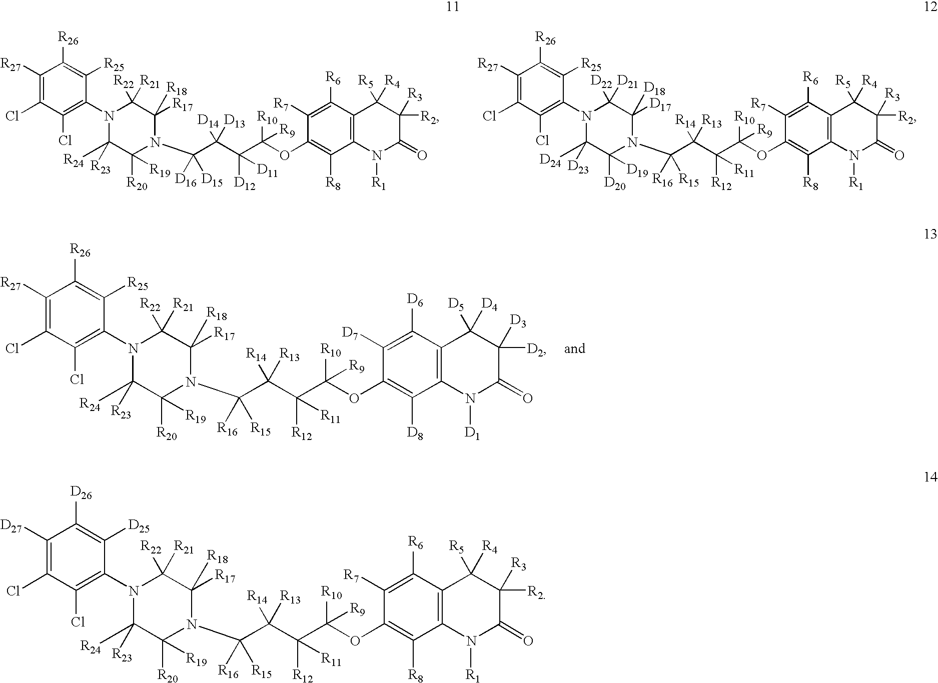

In certain embodiments, a deuterium-enriched compound of structural formula IV, wherein said compound is selected from the group consisting of:

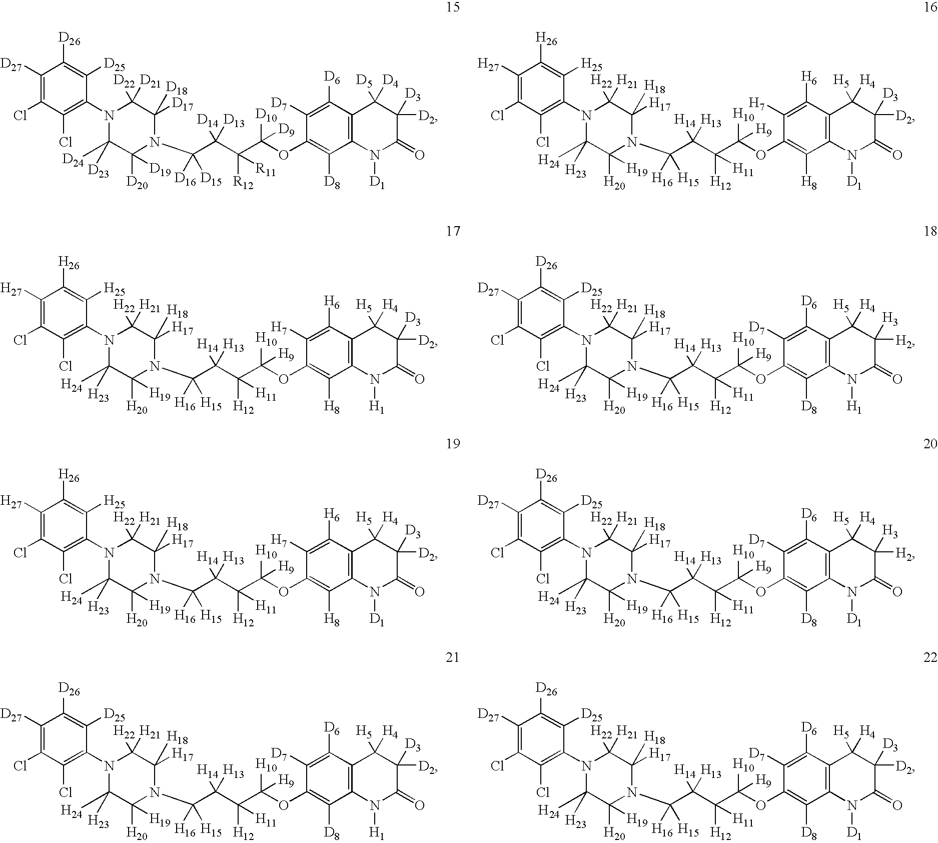

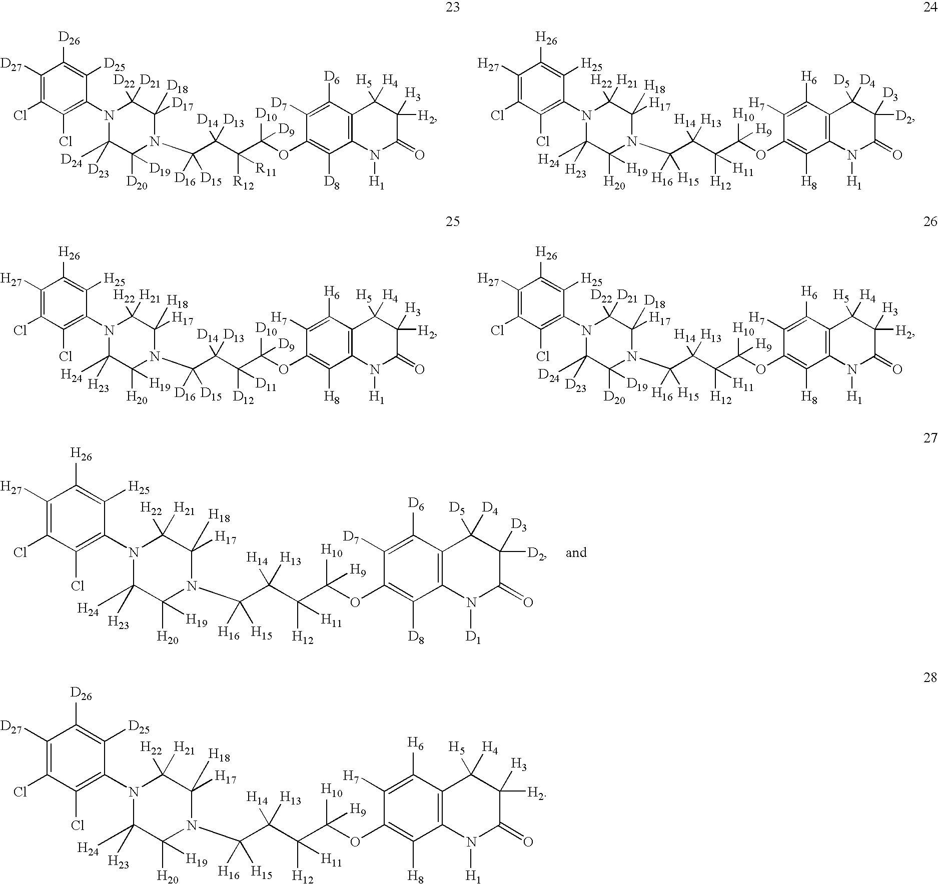

In certain embodiments, an isolated deuterium-enriched compound of structural formula IV or a pharmaceutically acceptable salt thereof:

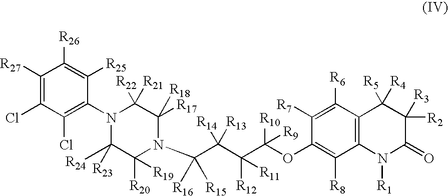

wherein R₁-R₂₇ are independently selected from H and D;

and the abundance of deuterium in R₁-R₂₇ is at least 4%.

In further embodiments, an isolated deuterium-enriched compound of structural formula IV, wherein the abundance of deuterium in R₁-R₂₇ is selected from at least 4%, at least 7%, at least 15%, at least 22%, at least 30%, at least 37%, at least 44%, at least 52%, at least 59%, at least 67%, at least 74%, at least 81%, at least 89%, at least 96%, and 100%.

In further embodiments, an isolated deuterium-enriched compound of structural formula IV, wherein the abundance of deuterium in R₁ is 100%.

In further embodiments, an isolated deuterium-enriched compound of structural formula IV, wherein the abundance of deuterium in R₂-R₃ is selected from at least 50% and 100%.

In further embodiments, an isolated deuterium-enriched compound of structural formula IV, wherein the abundance of deuterium in R₆-R₈ and R₂₅-R₂₇ is selected from at least 17%, at least 33%, at least 50%, at least 67%, at least 83%, and 100%.

In further embodiments, an isolated deuterium-enriched compound of structural formula IV, wherein the abundance of deuterium in R₁ and R₂-R₃ is selected from at least 33%, at least 67%, and 100%.

In further embodiments, an isolated deuterium-enriched compound of structural formula IV, wherein the abundance of deuterium in R₁, R₆-R₈, and R₂₅-R₂₇ is selected from at least 14%, at least 29%, at least 43%, at least 57%, at least 71%, at least 86%, and 100%.

In further embodiments, an isolated deuterium-enriched compound of structural formula IV, wherein the abundance of deuterium in R₂-R₃, R₆-R₈, and R₂₅-R₂₇ is selected from at least 13%, at least 25%, at least 38%, at least 50%, at least 63%, at least 75%, at least 88%, and 100%.

In further embodiments, an isolated deuterium-enriched compound of structural formula IV, wherein the abundance of deuterium in R₁, R₂-R₃, R₆-R₈, and R₂₅-R₂₇ is selected from at least 11%, at least 22%, at least 33%, at least 44%, at least 56%, at least 67%, at least 78%, and 100%.

In further embodiments, an isolated deuterium-enriched compound of structural formula IV, wherein the abundance of deuterium in R₆-R₂₇ is selected from at least 5%, at least 9%, at least 14%, at least 18%, at least 23%, at least 27%, at least 32%, at least 36%, at least 41%, at least 45%, at least 50%, at least 55%, at least 59%, at least 64%, at least 68%, at least 73%, at least 77%, at least 82%, at least 86%, at least 91%, at least 95%, and 100%.

In further embodiments, an isolated deuterium-enriched compound of structural formula IV, wherein the abundance of deuterium in R₂-R₅ is selected from at least 25%, at least 50%, at least 75%, and 100%.

In further embodiments, an isolated deuterium-enriched compound of structural formula IV, wherein the abundance of deuterium in R₂-R₃ is selected from at least 50% and 100%.

In further embodiments, an isolated deuterium-enriched compound of structural formula IV, wherein the abundance of deuterium in R₆-R₈ and R₂₅-R₂₇ is selected from at least 17%, at least 33%, at least 50%, at least 67%, at least 83%, and 100%.

In further embodiments, an isolated deuterium-enriched compound of structural formula IV, wherein the abundance of deuterium in R₁ and R₂-R₃ is selected from at least 33%, at least 67%, and 100%.

In further embodiments, an isolated deuterium-enriched compound of structural formula IV, wherein the abundance of deuterium in R₁, R₆-R₈, and R₂₅-R₂₇ is selected from at least 14%, at least 29%, at least 43%, at least 57%, at least 71%, at least 86%, and 100%.

In further embodiments, an isolated deuterium-enriched compound of structural formula IV, wherein the abundance of deuterium in R₂-R₃, R₆-R₈, and R₂₅-R₂₇ is selected from at least 13%, at least 25%, at least 38%, at least 50%, at least 63%, at least 75%, at least 88%, and 100%.

In further embodiments, an isolated deuterium-enriched compound of structural formula IV, wherein the abundance of deuterium in R₁, R₂-R₃, R₆-R₈, and R₂₅-R₂₇ is selected from at least 11%, at least 22%, at least 33%, at least 44%, at least 56%, at least 67%, at least 78%, and 100%.

In further embodiments, an isolated deuterium-enriched compound of structural formula IV, wherein the abundance of deuterium in R₆-R₂₇ is selected from at least 5%, at least 9%, at least 14%, at least 18%, at least 23%, at least 27%, at least 32%, at least 36%, at least 41%, at least 45%, at least 50%, at least 55%, at least 59%, at least 64%, at least 68%, at least 73%, at least 77%, at least 82%, at least 86%, at least 91%, at least 95%, and 100%.

In further embodiments, an isolated deuterium-enriched compound of structural formula IV, wherein the abundance of deuterium in R₂-R₅ is selected from at least 25%, at least 50%, at least 75%, and 100%.

In yet further embodiments, an isolated deuterium-enriched compound of structural formula IV, wherein the abundance of deuterium in R₉-R₁₆ is selected from at least 13%, at least 25%, at least 38%, at least 50%, at least 63%, at least 75%, at least 88%, and 100%.

In yet even further embodiments, an isolated deuterium-enriched compound of structural formula IV, wherein the abundance of deuterium in R₁₇-R₂₄ is selected from at least 13%, at least 25%, at least 38%, at least 50%, at least 63%, at least 75%, at least 88%, and 100%.

In certain embodiments, an isolated deuterium-enriched compound of structural formula IV, wherein the compound is selected from the group consisting of:

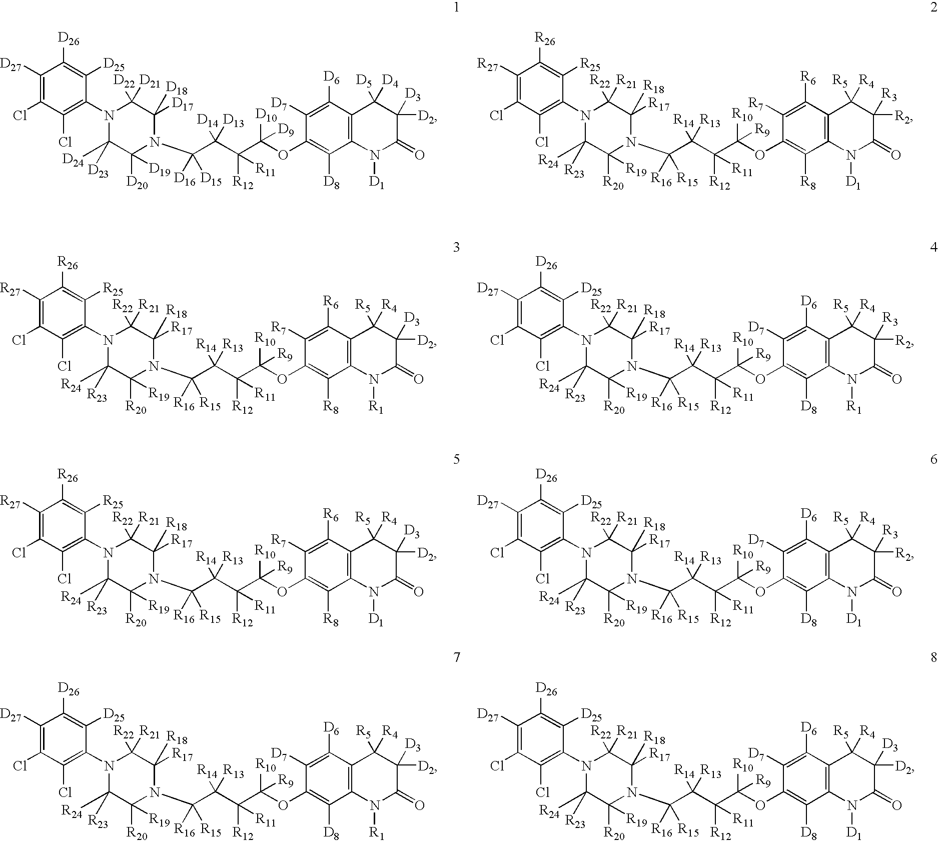

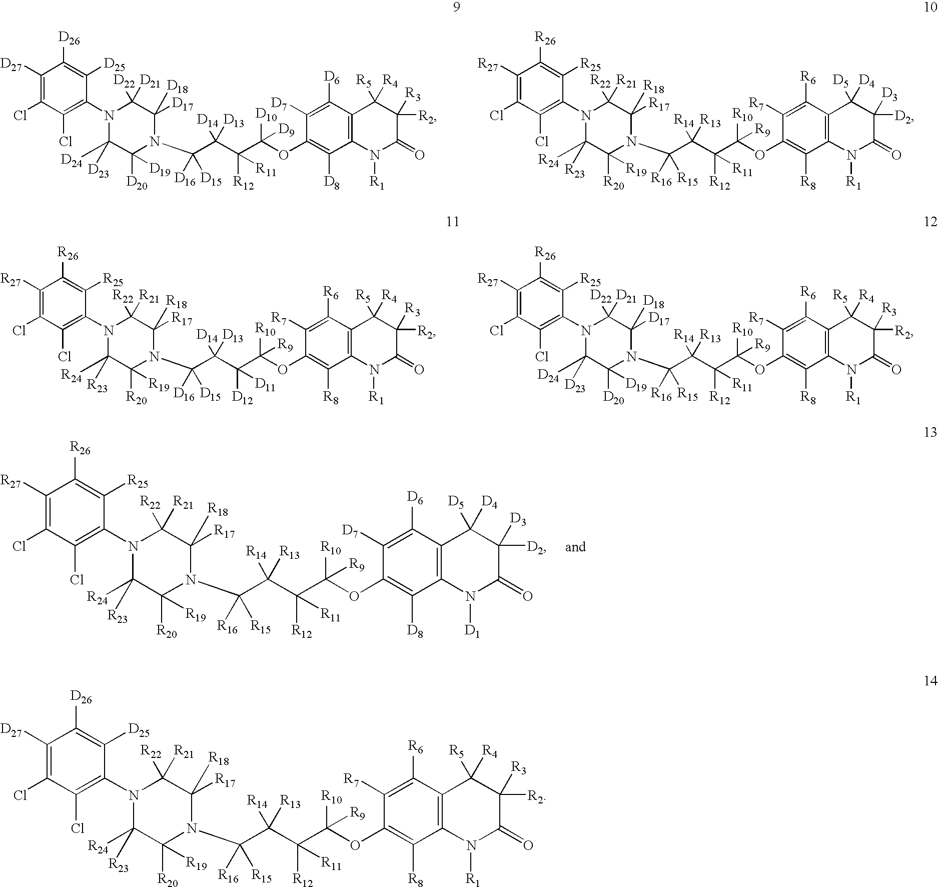

In certain embodiments, an isolated deuterium-enriched compound of structural formula IV, wherein the compound is selected from the group consisting of:

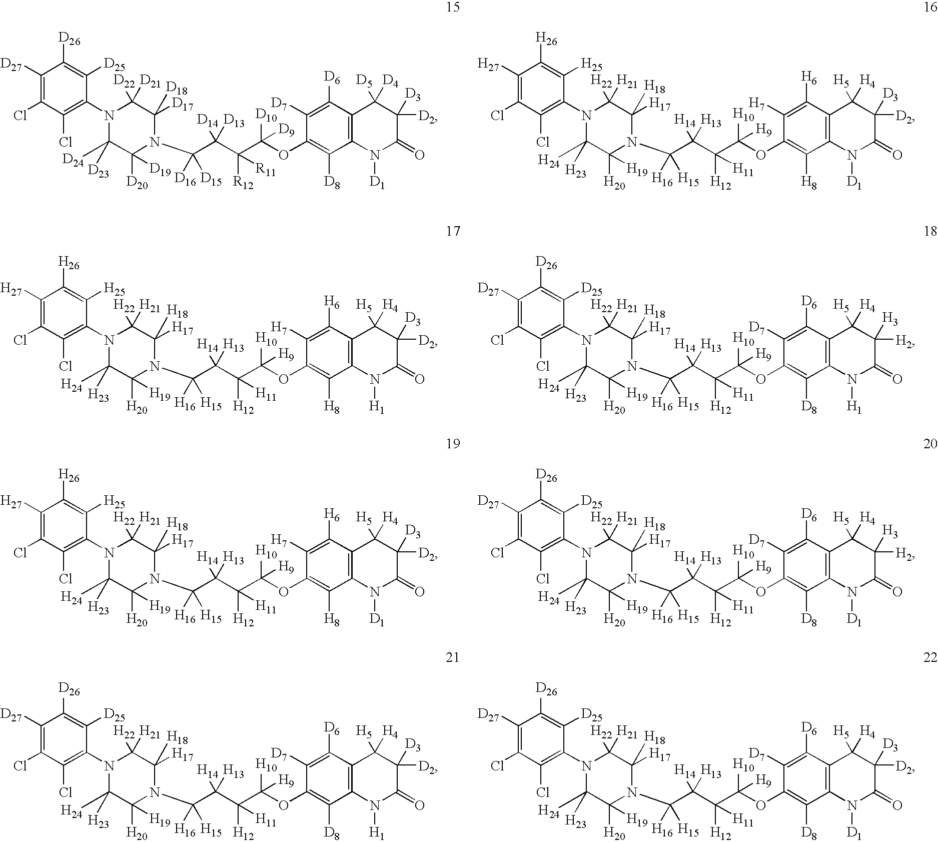

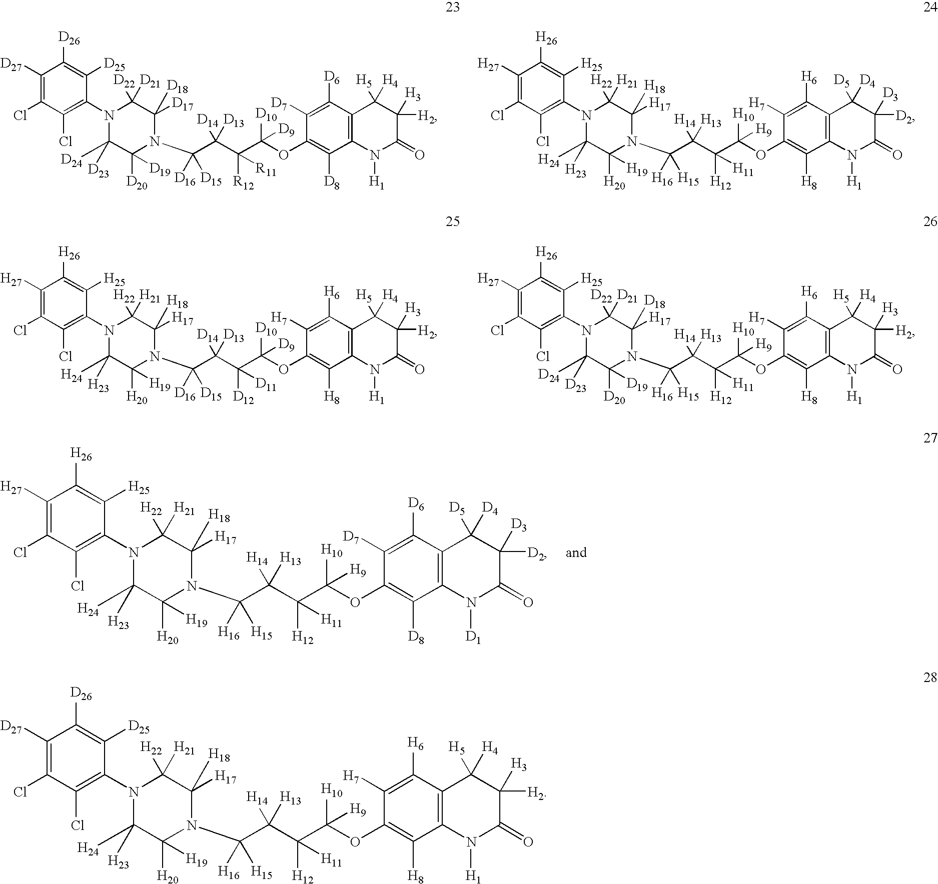

In certain embodiments, a mixture of deuterium-enriched compounds of formula IV or a pharmaceutically acceptable salt thereof:

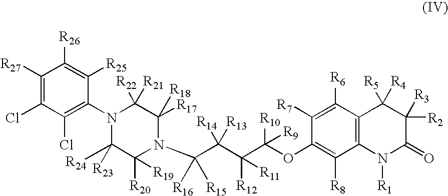

wherein R₁-R₂₇ are independently selected from H and D;

and the abundance of deuterium in R₁-R₂₇ is at least 4%.

In further embodiments, a mixture of deuterium-enriched compounds of structural formula IV, wherein the abundance of deuterium in R₁-R₂₇ is selected from at least 4%, at least 7%, at least 15%, at least 22%, at least 30%, at least 37%, at least 44%, at least 52%, at least 59%, at least 67%, at least 74%, at least 81%, at least 89%, at least 96%, and 100%.

In further embodiments, a mixture of deuterium-enriched compounds of structural formula IV, wherein the abundance of deuterium in R₁ is 100%.

In further embodiments, a mixture of deuterium-enriched compounds of structural formula IV, wherein the abundance of deuterium in R₂-R₃ is selected from at least 50% and 100%.

In further embodiments, a mixture of deuterium-enriched compounds of structural formula IV, wherein the abundance of deuterium in R₆-R₈ and R₂₅-R₂₇ is selected from at least 17%, at least 33%, at least 50%, at least 67%, at least 83%, and 100%.

In further embodiments, a mixture of deuterium-enriched compounds of structural formula IV, wherein the abundance of deuterium in R₁ and R₂-R₃ is selected from at least 33%, at least 67%, and 100%.

In further embodiments, a mixture of deuterium-enriched compounds of structural formula IV, wherein the abundance of deuterium in R₁, R₆-R₈, and R₂₅-R₂₇ is selected from at least 14%, at least 29%, at least 43%, at least 57%, at least 71%, at least 86%, and 100%.

In further embodiments, a mixture of deuterium-enriched compounds of structural formula IV, wherein the abundance of deuterium in R₂-R₃, R₆-R₈, and R₂₅-R₂₇ is selected from at least 13%, at least 25%, at least 38%, at least 50%, at least 63%, at least 75%, at least 88%, and 100%.

In further embodiments, a mixture of deuterium-enriched compounds of structural formula IV, wherein the abundance of deuterium in R₁, R₂-R₃, R₆-R₈, and R₂₅-R₂₇ is selected from at least 11%, at least 22%, at least 33%, at least 44%, at least 56%, at least 67%, at least 78%, and 100%.

In further embodiments, a mixture of deuterium-enriched compounds of structural formula IV, wherein the abundance of deuterium in R₆-R₂₇ is selected from at least 5%, at least 9%, at least 14%, at least 18%, at least 23%, at least 27%, at least 32%, at least 36%, at least 41%, at least 45%, at least 50%, at least 55%, at least 59%, at least 64%, at least 68%, at least 73%, at least 77%, at least 82%, at least 86%, at least 91%, at least 95%, and 100%.

In further embodiments, a mixture of deuterium-enriched compounds of structural formula IV, wherein the abundance of deuterium in R₂-R₅ is selected from at least 25%, at least 50%, at least 75%, and 100%.

In yet further embodiments, a mixture of deuterium-enriched compounds of structural formula IV, wherein the abundance of deuterium in R₉-R₁₆ is selected from at least 13%, at least 25%, at least 38%, at least 50%, at least 63%, at least 75%, at least 88%, and 100%.

In yet even further embodiments, a mixture of deuterium-enriched compounds of structural formula IV, wherein the abundance of deuterium in R₁₇-R₂₄ is selected from at least 13%, at least 25%, at least 38%, at least 50%, at least 63%, at least 75%, at least 88%, and 100%.

In certain embodiments, a mixture of deuterium-enriched compounds of structural formula IV, wherein the compounds are selected from the group consisting of:

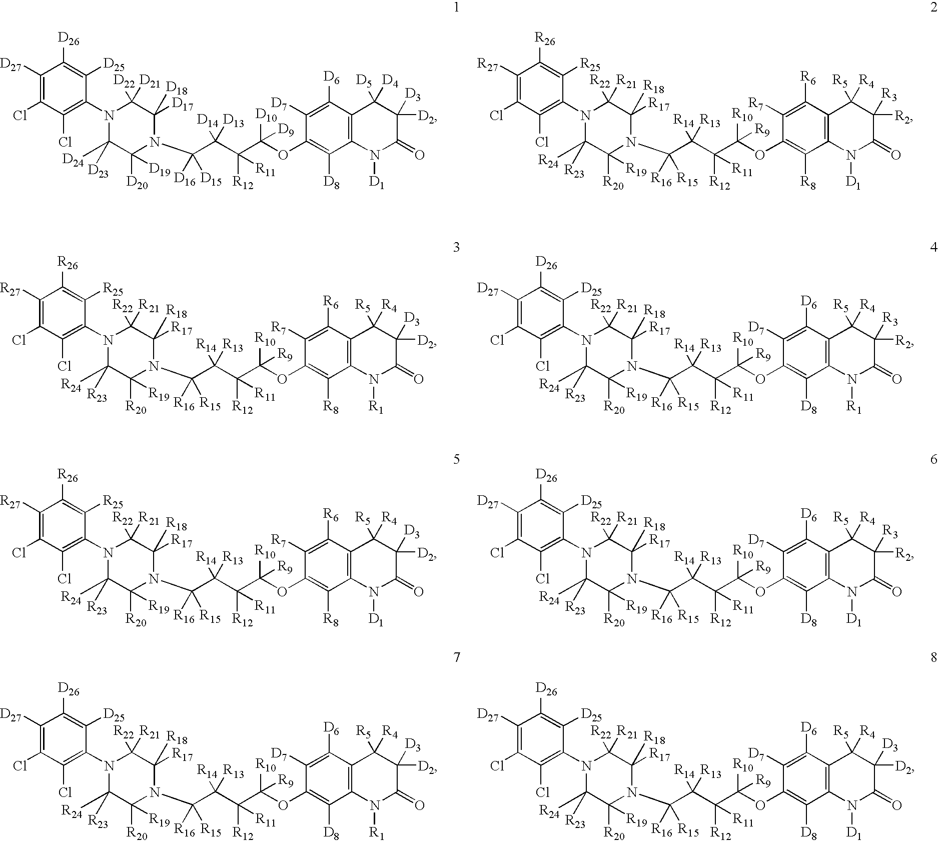

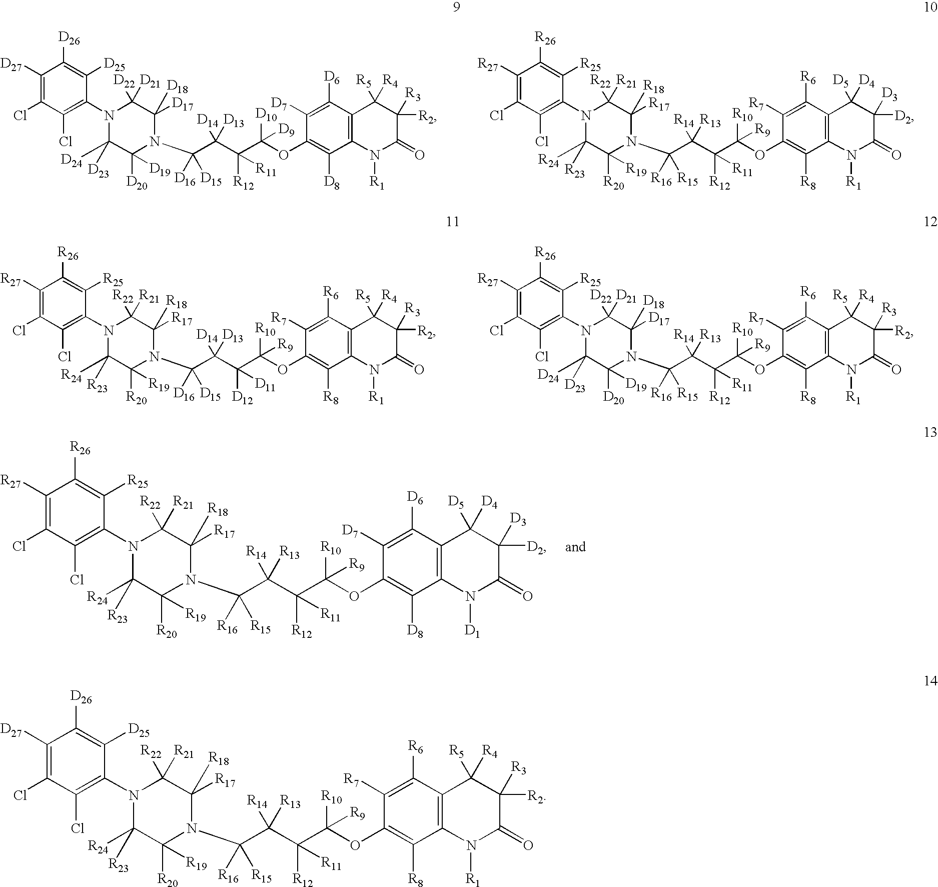

In certain embodiments, mixture of deuterium-enriched compounds of structural formula IV, wherein the compounds are selected from the group consisting of:

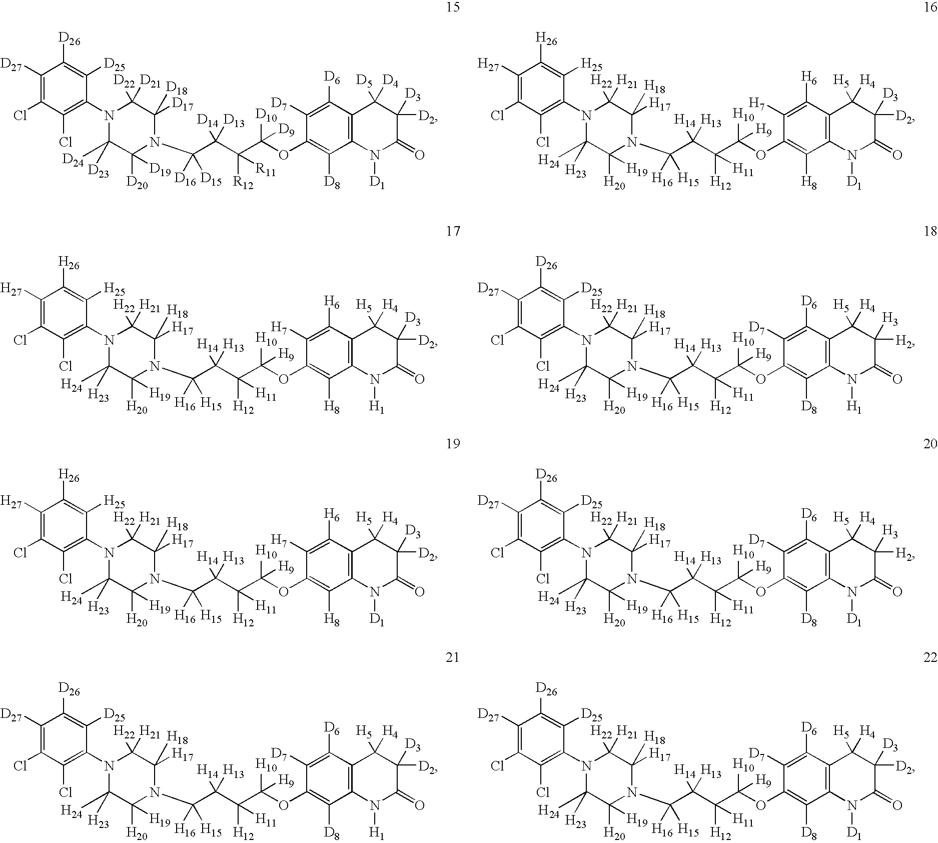

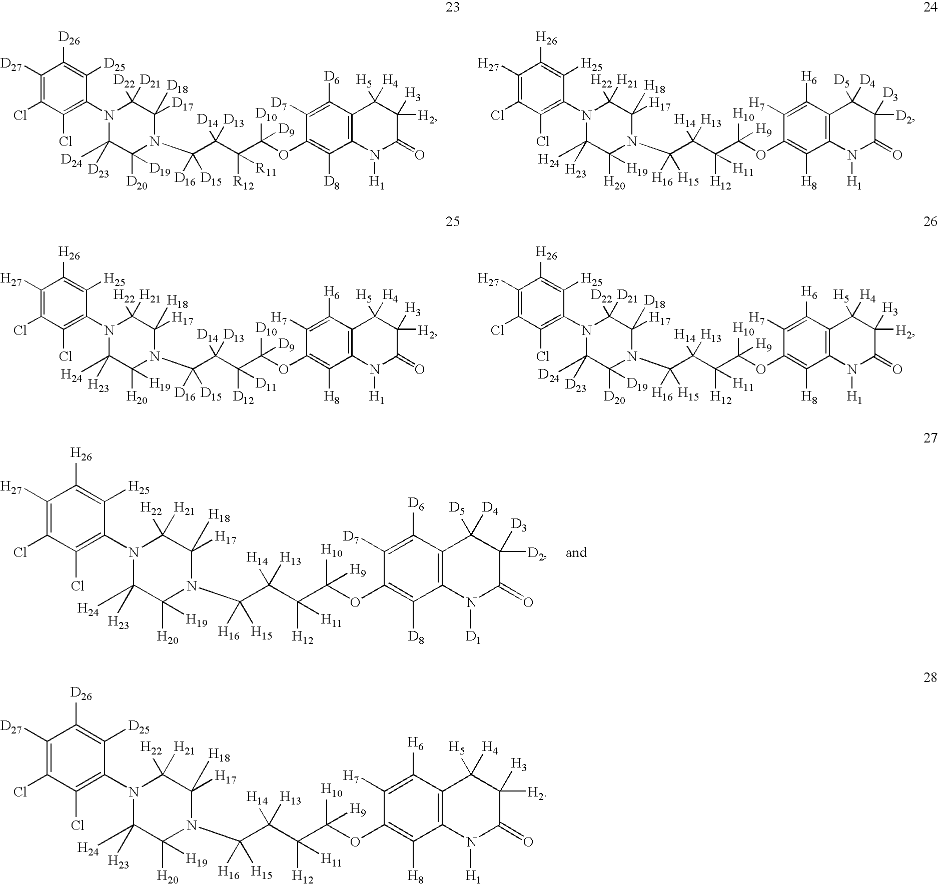

In certain embodiments, a pharmaceutical composition, comprising: a pharmaceutically acceptable carrier and a therapeutically effective amount of a compound of structural formula IV, or a pharmaceutically acceptable salt form thereof. In certain embodiments, a method for treating schizophrenia comprising: administering, to a patient in need thereof, a therapeutically effective amount of a compound of structural formula IV, or a pharmaceutically acceptable salt form thereof.

All publications and references cited herein are expressly incorporated herein by reference in their entirety. However, with respect to any similar or identical terms found in both the incorporated publications or references and those explicitly put forth or defined in this document, then those terms definitions or meanings explicitly put forth in this document shall control in all respects.

As used herein, the terms below have the meanings indicated.

The singular forms “a,” “an,” and “the” may refer to plural articles unless specifically stated otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

When ranges of values are disclosed, and the notation “from n₁ . . . to n₂” or “n₁-n₂” is used, where n₁ and n₂ are the numbers, then unless otherwise specified, this notation is intended to include the numbers themselves and the range between them. This range may be integral or continuous between and including the end values.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

The term “is/are deuterium,” when used to describe a given position in a molecule such as R₁-R₂₇ or the symbol “D,” when used to represent a given position in a drawing of a molecular structure, means that the specified position is enriched with deuterium above the naturally occurring distribution of deuterium. In one embodiment deuterium enrichment is no less than about 1%, in another no less than about 5%, in another no less than about 10%, in another no less than about 20%, in another no less than about 50%, in another no less than about 70%, in another no less than about 80%, in another no less than about 90%, or in another no less than about 98% of deuterium at the specified position.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disorder” as used herein is intended to be generally synonymous, and is used interchangeably with, the terms “disease” and “condition” (as in medical condition), in that all reflect an abnormal condition of the human or animal body or of one of its parts that impairs normal functioning, is typically manifested by distinguishing signs and symptoms.

The terms “treat,” “treating,” and “treatment” are meant to include alleviating or abrogating a disorder or one or more of the symptoms associated with a disorder; or alleviating or eradicating the cause(s) of the disorder itself. As used herein, reference to “treatment” of a disorder is intended to include prevention. The terms “prevent,” “preventing,” and “prevention” refer to a method of delaying or precluding the onset of a disorder; and/or its attendant symptoms, barring a subject from acquiring a disorder or reducing a subject's risk of acquiring a disorder.

The term “therapeutically effective amount” refers to the amount of a compound that, when administered, is sufficient to prevent development of, or alleviate to some extent, one or more of the symptoms of the disorder being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein in reference, for example, to a mammalian subject, such as a human patient.

The term “combination therapy” means the administration of two or more therapeutic agents to treat a therapeutic disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the disorders described herein.

The term “D2 receptor,” refers to a subclass of metabotropic G-protein-coupled receptors and/or transporters found extensively in the central nervous system, for which the neurotransmitter dopamine is the primary endogenous ligand. At least five different subtypes (D1, D2, D3, D4, and D5) of dopamine receptors are known. D2 receptor activation is coupled to the G protein G_{αi}, which directly inhibits the formation of cAMP by inhibiting the enzyme adenylate cyclase. Decreased cAMP in neurons is typically inhibitory. Dysfunction of dopaminergic neurotransmission in the central nervous system has been implicated in a variety of neuropsychiatric disorders, including social phobia, Tourette's syndrome, Parkinson's disease, schizophrenia, neuroleptic malignant syndrome, attention-deficit hyperactivity disorder (ADHD), and drug and alcohol dependence. D2-receptor ligands may exhibit functional selectivity, i.e., modulation of D2 receptors by different ligands may activate different signal transduction pathways.

The term “5HT1A receptor,” refers to a subclass of a family of receptors for the neurotransmitter and peripheral signal mediator serotonin. The 5-HT1A receptor is coupled to an intracellular G-protein (Gᵢ/Gₒ) which inhibits the formation of cAMP by inhibiting the enzyme adenylate cyclase. 5-HT1A acts on the central nervous system, where it induces neuronal inhibition and regulates various behaviours, including sleep, feeding, thermoregulation, aggression, and anxiety.

The term “5HT2A receptor,” refers to a subclass of a family of receptors for the neurotransmitter and peripheral signal mediator serotonin. 5-HT2 receptors mediate many of the central and peripheral physiologic functions of serotonin. Cardiovascular effects include vasoconstriction and platelet aggregation. Central nervous system effects include neuronal sensitization to tactile stimuli and mediation of hallucinogenic effects of phenylisopropylamine hallucinogens. Members of the 5-HT2 subclass include 5-HT2A, 5-HT2B, and 5-HT2C. The 5-HT2A receptor is known primarily to couple to the G_{αq} signal transduction pathway. Upon receptor stimulation with agonist, G_{αq} and beta-gamma subunits dissociate to initiate downstream effector pathways. G_{αq} stimulates phospholipase C (PLC) activity, which subsequently promotes the release of diacylglycerol (DAG) and inositol triphosphate (IP3), which in turn stimulate protein kinase C (PKC) activity and Ca²⁺ release. Effects of 5-HT2A activation in the central nervous system include neuronal excitation, which mediates a number of behavioural effects, including anxiety. 5-HT2A-receptor ligands may exhibit functional selectivity, i.e., modulation of 5-HT2A receptors by different ligands may activate different signal transduction pathways.

The term “D2 receptor -mediated disorder,” refers to a disorder that is characterized by abnormal D2 receptor activity. A D2 receptor-mediated disorder may be completely or partially mediated by modulating D2 receptors. In particular, a D2 receptor-mediated disorder is one in which modulation of D2 receptors results in some effect on the underlying disorder e.g., administration of a D2 receptor modulator results in some improvement in at least some of the patients being treated.

The term “5-HT1A receptor -mediated disorder,” refers to a disorder that is characterized by abnormal 5-HT1A receptor activity. A 5-HT1A receptor-mediated disorder may be completely or partially mediated by modulating 5-HT1A receptors. In particular, a 5-HT1A receptor-mediated disorder is one in which modulation of 5-HT1A receptors results in some effect on the underlying disorder e.g., administration of a 5-HT1A receptor modulator results in some improvement in at least some of the patients being treated.

The term “5-HT2A receptor-mediated disorder,” refers to a disorder that is characterized by abnormal 5-HT2A receptor activity. A 5-HT2A receptor-mediated disorder may be completely or partially mediated by modulating 5-HT2A receptors. In particular, a 5-HT2A receptor-mediated disorder is one in which modulation of 5-HT2A receptors results in some effect on the underlying disorder e.g., administration of a 5-HT2A receptor modulator results in some improvement in at least some of the patients being treated.

The term “D2 receptor modulator,” refers to the ability of a compound disclosed herein to alter the function of D2 receptors. A modulator may activate the activity of a D2 receptor, may activate or inhibit the activity of a D2 receptor depending on the concentration of the compound exposed to the D1 receptor, or may inhibit the activity of a D2 receptor. Such activation or inhibition may be contingent on the occurrence of a specific event, such as activation of a signal transduction pathway, and/or may be manifest only in particular cell types. The term “modulate” or “modulation” also refers to altering the function of a D2 receptor by increasing or decreasing the probability that a complex forms between a D2 receptor and a natural binding partner. A modulator may increase the probability that such a complex forms between the D2 receptor and the natural binding partner, may increase or decrease the probability that a complex forms between the D2 receptor and the natural binding partner depending on the concentration of the compound exposed to the D2 receptor, and or may decrease the probability that a complex forms between the D2 receptor and the natural binding partner.

The term “5-HT1A receptor modulator,” refers to the ability of a compound disclosed herein to alter the function of 5-HT1A receptors. A modulator may activate the activity of a 5-HT1A receptor, may activate or inhibit the activity of a 5-HT1A receptor depending on the concentration of the compound exposed to the 5-HT1A receptor, or may inhibit the activity of a 5-HT1A receptor. Such activation or inhibition may be contingent on the occurrence of a specific event, such as activation of a signal transduction pathway, and/or may be manifest only in particular cell types. The term “modulate” or “modulation” also refers to altering the function of a 5-HT1A receptor by increasing or decreasing the probability that a complex forms between a 5-HT1A receptors and a natural binding partner. A modulator may increase the probability that such a complex forms between the 5-HT1A receptor and the natural binding partner, may increase or decrease the probability that a complex forms between the 5-HT1A receptor and the natural binding partner depending on the concentration of the compound exposed to the 5-HT1A receptor, and or may decrease the probability that a complex forms between the 5-HT1A receptor and the natural binding partner.

The term “5-HT2A receptor modulator,” refers to the ability of a compound disclosed herein to alter the function of 5-HT2A receptors. A modulator may activate the activity of a 5-HT2A receptor, may activate or inhibit the activity of a 5-HT2A receptor depending on the concentration of the compound exposed to the 5-HT2A receptor, or may inhibit the activity of a 5-HT2A receptor. Such activation or inhibition may be contingent on the occurrence of a specific event, such as activation of a signal transduction pathway, and/or may be manifest only in particular cell types. The term “modulate” or “modulation” also refers to altering the function of a 5-HT2A receptor by increasing or decreasing the probability that a complex forms between a 5-HT2A receptor and a natural binding partner. A modulator may increase the probability that such a complex forms between the 5-HT2A receptor and the natural binding partner, may increase or decrease the probability that a complex forms between the 5-HT2A receptor and the natural binding partner depending on the concentration of the compound exposed to the 5-HT2A receptor, and or may decrease the probability that a complex forms between the 5-HT2A receptor and the natural binding partner.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenecity, are commensurate with a reasonable benefit/risk ratio, and are effective for their intended use.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenecity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The terms “active ingredient,” “active compound,” and “active substance” refer to a compound, which is administered, alone or in combination with one or more pharmaceutically acceptable excipients or carriers, to a subject for treating, preventing, or ameliorating one or more symptoms of a disorder.

The terms “drug,” “therapeutic agent,” and “chemotherapeutic agent” refer to a compound, or a pharmaceutical composition thereof, which is administered to a subject for treating, preventing, or ameliorating one or more symptoms of a disorder.

The term “release controlling excipient” refers to an excipient whose primary function is to modify the duration or place of release of the active substance from a dosage form as compared with a conventional immediate release dosage form.

The term “nonrelease controlling excipient” refers to an excipient whose primary function do not include modifying the duration or place of release of the active substance from a dosage form as compared with a conventional immediate release dosage form.

The term “prodrug” refers to a compound functional derivative of the compound as disclosed herein and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev.1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

The compounds disclosed herein can exist as therapeutically acceptable salts. The term “therapeutically acceptable salt,” as used herein, represents salts or zwitterionic forms of the compounds disclosed herein which are therapeutically acceptable as defined herein. The salts can be prepared during the final isolation and purification of the compounds or separately by reacting the appropriate compound with a suitable acid or base. Therapeutically acceptable salts include acid and basic addition salts. For a more complete discussion of the preparation and selection of salts, refer to “Handbook of Pharmaceutical Salts, Properties, and Use,” Stah and Wermuth, Ed.; (Wiley-VCH and VHCA, Zurich, 2002) and Berge et al., J. Pharm. Sci. 1977, 66, 1-19.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

While it may be possible for the compounds of the subject invention to be administered as the raw chemical, it is also possible to present them as a pharmaceutical composition. Accordingly, provided herein are pharmaceutical compositions which comprise one or more of certain compounds disclosed herein, or one or more pharmaceutically acceptable salts, prodrugs, or solvates thereof, together with one or more pharmaceutically acceptable carriers thereof and optionally one or more other therapeutic ingredients. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The compositions include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The compositions may conveniently be presented in unit dosage form and may be prepared by any of the methods well known in the art of pharmacy. Typically, these methods include the step of bringing into association a compound of the subject invention or a pharmaceutically salt, prodrug, or solvate thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

Disclosed herein are methods of treating a D2 receptor-mediated disorder, a 5-HT1A receptor-mediated disorder, and/or a 5-HT2A receptor-mediated disorder comprising administering to a subject having or suspected to have such a disorder, a therapeutically effective amount of a compound as disclosed herein or a pharmaceutically acceptable salt, solvate, or prodrug thereof.

D2 receptor, 5-HT1A receptor, and/or 5-HT2A receptor-mediated disorders, include, but are not limited to, attention deficit hyperactivity disorder, autism, drug dependence, acute bipolar mania, bipolar disorder, and major depressive disorder, and/or any disorder which can lessened, alleviated, or prevented by administering a D2 receptor modulator, 5-HT1A receptor modulator, and/or a 5-HT2A receptor modulator.

In certain embodiments, a method of treating a D2 receptor-mediated disorder, a 5-HT1A receptor-mediated disorder, and/or a 5-HT2A receptor-mediated disorder comprises administering to the subject a therapeutically effective amount of a compound of as disclosed herein, or a pharmaceutically acceptable salt, solvate, or prodrug thereof, so as to affect: (1) decreased inter-individual variation in plasma levels of the compound or a metabolite thereof; (2) increased average plasma levels of the compound or decreased average plasma levels of at least one metabolite of the compound per dosage unit; (3) decreased inhibition of, and/or metabolism by at least one cytochrome P₄₅₀ or monoamine oxidase isoform in the subject; (4) decreased metabolism via at least one polymorphically-expressed cytochrome P₄₅₀ isoform in the subject; (5) at least one statistically-significantly improved disorder-control and/or disorder-eradication endpoint; (6) an improved clinical effect during the treatment of the disorder, (7) prevention of recurrence, or delay of decline or appearance, of abnormal alimentary or hepatic parameters as the primary clinical benefit, or (8) reduction or elimination of deleterious changes in any diagnostic hepatobiliary function endpoints, as compared to the corresponding non-isotopically enriched compound.

In certain embodiments, inter-individual variation in plasma levels of the compounds as disclosed herein, or metabolites thereof, is decreased; average plasma levels of the compound as disclosed herein are increased; average plasma levels of a metabolite of the compound as disclosed herein are decreased; inhibition of a cytochrome P₄₅₀ or monoamine oxidase isoform by a compound as disclosed herein is decreased; or metabolism of the compound as disclosed herein by at least one polymorphically-expressed cytochrome P₄₅₀ isoform is decreased; by greater than about 5%, greater than about 10%, greater than about 20%, greater than about 30%, greater than about 40%, or by greater than about 50% as compared to the corresponding non-isotopically enriched compound.

Plasma levels of the compound as disclosed herein, or metabolites thereof, may be measured using the methods described by Li et al. Rapid Communications in Mass Spectrometry 2005, 19, 1943-1950, Hughes et al, Xenobiotica 1992, 22(7), 859-69, Varma et al, Journal of Pharmaceutical and Biomedical Analysis 2004, 36(3), 669-674, Massoud et al, Journal of Chromatography, B: Biomedical Sciences and Applications 1999, 734(1), 163-167, Kim et al, Journal of Pharmaceutical and Biomedical Analysis 2003, 31(2), 341-349, and Lindeke et al, Acta Pharmaceutica Suecica 1981, 18(1), 25-34.

Examples of cytochrome P₄₅₀ isoforms in a mammalian subject include, but are not limited to, CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

Examples of monoamine oxidase isoforms in a mammalian subject include, but are not limited to, MAO_{A}, and MAO_{B}.

The inhibition of the cytochrome P₄₅₀ isoform is measured by the method of Ko et al. (British Journal of Clinical Pharmacology, 2000, 49, 343-35 1). The inhibition of the MAO_{A} isoform is measured by the method of Weyler et al. (J. Biol Chem. 1985, 260, 13199-13207). The inhibition of the MAO_{B} isoform is measured by the method of Uebelhack et al. (Pharmacopsychiatry, 1998, 31, 187-192).

Examples of polymorphically-expressed cytochrome P₄₅₀ isoforms in a mammalian subject include, but are not limited to, CYP2C8, CYP2C9, CYP2C19, and CYP2D6.

The metabolic activities of liver microsomes, cytochrome P₄₅₀ isoforms, and monoamine oxidase isoforms are measured by the methods described herein.

Examples of improved disorder-control and/or disorder-eradication endpoints, or improved clinical effects include, but are not limited to, improved positive and negative syndrome scale (PANSS) total scores, reduced incidence of extrapyrimidal disorder, reduced incidence of somnolence, reduced incidence of tremor, delayed time to relapse in bipolar disorder, improved investigator's assessment questionnaire (IAQ) scores, improved Montgomery-Asberg depression rating scale (MADRS) scores, and improved self-rating depression (SDS) scores. Drug Report for Aripiprazole, Thompson Investigational Drug Database, (Aug. 12, 2008); Fleischhacker, Expert Opin. Pharmacother. 6(12) 2091-2101 (2005); and Sanford et al., CNS Drugs 22(4) 335-52 (2008).

Examples of diagnostic hepatobiliary function endpoints include, but are not limited to, alanine aminotransferase (“ALT”), serum glutamic-pyruvic transaminase (“SGPT”), aspartate aminotransferase (“AST” or “SGOT”), ALT/AST ratios, serum aldolase, alkaline phosphatase (“ALP”), ammonia levels, bilirubin, gamma-glutamyl transpeptidase (“GGTP,” “γ-GTP,” or “GGT”), leucine aminopeptidase (“LAP”), liver biopsy, liver ultrasonography, liver nuclear scan, 5′-nucleotidase, and blood protein. Hepatobiliary endpoints are compared to the stated normal levels as given in “Diagnostic and Laboratory Test Reference”, 4th edition, Mosby, 1999. These assays are run by accredited laboratories according to standard protocol.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### Combination Therapy

The compounds disclosed herein may also be combined or used in combination with other agents useful in the treatment of D2 receptor-mediated disorders, 5-HT1A receptor-mediated disorders, and/or 5-HT2A receptor-mediated disorders. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required.

In certain embodiments, the compounds disclosed herein can be combined with one or more antidepressants and antipsychotics.

In further embodiments, the compounds disclosed herein can be combined with an antidepressant selected from the group consisting of citalopram, escitalopram, paroxetine, fluotexine, fluvoxamine, sertraline, isocarboxazid, moclobemide, phenelzine, tranylcypromine, amitriptyline, clomipramine, desipramine, dosulepin, imipramine, nortriptyline, protriptyline, trimipramine, lofepramine, maprotiline, amoxapine, mianserin, mirtazapine, duloxetine, nefazodone, reboxetine, trazodone, venlafaxine, tianeptine, and milnacipran.

In further embodiments, the compounds disclosed herein can be combined with an antipsychotic selected from the group consisting of haloperidol, chlorpromazine, fluphenazine, perphenazine, prochlorperazine, thioridazine, trifluoperazine, mesoridazine, promazine, triflupromazine, levomepromazine, promethazine, chlorprothixene, flupenthixol, thiothixene, zuclopenthixol, clozapine, olanzapine, quetiapine, ziprasidone, amisulpride, paliperidone, bifeprunox, norclozapine, risperidone, tetrabenazine, and cannabidiol.

In further embodiments, the compounds disclosed herein can be combined with lithium or valproate.

The compounds disclosed herein can also be administered in combination with other classes of compounds, including, but not limited to, norepinephrine reuptake inhibitors (NRIs) such as atomoxetine; dopamine reuptake inhibitors (DARIs), such as methylphenidate; serotonin-norepinephrine reuptake inhibitors (SNRIs), such as milnacipran; sedatives, such as diazepham; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; opioids, such as tramadol; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-muscarinic agents; beta-muscarinic agents, such as carvedilol and metoprolol; antiarrhythmic agents; diuretics, such as chlorothlazide, hydrochiorothiazide, flumethiazide, hydroflumethiazide, bendroflumethiazide, methylchlorothiazide, trichioromethiazide, polythiazide, benzothlazide, ethacrynic acid, tricrynafen, chlorthalidone, furosenilde, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stablizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

Thus, in another aspect, certain embodiments provide methods for treating D2 receptor-mediated disorders, 5-HT1A receptor-mediated disorders, and/or 5-HT2A receptor-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of D2 receptor-mediated disorders, 5-HT1A receptor-mediated disorders, and/or 5-HT2A receptor-mediated disorders.

### General Synthetic Methods for Preparing Compounds

Isotopic hydrogen can be introduced into a compound as disclosed herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. Exchange techniques, on the other hand, may yield lower tritium or deuterium incorporation, often with the isotope being distributed over many sites on the molecule.

The compounds as disclosed herein can be prepared by methods known to one of skill in the art and routine modifications thereof, and/or following procedures similar to those described in the Example section herein and routine modifications thereof, and/or procedures found in EP 0367141, US 20060079690, US 20060258869, US 20070213535, US 20070238876, WO 2003/026659, WO 2004/063162, WO 2004/105682, WO 2007/094009, WO 2007/118923, WO 2007/148191, and WO 2008/001188, which are hereby incorporated in their entirety, and references cited therein and routine modifications thereof. Compounds as disclosed herein can also be prepared as shown in any of the following schemes and routine modifications thereof.

The following schemes can be used to practice the present invention. Any position shown as deuterium may be optionally substituted with deuterium.

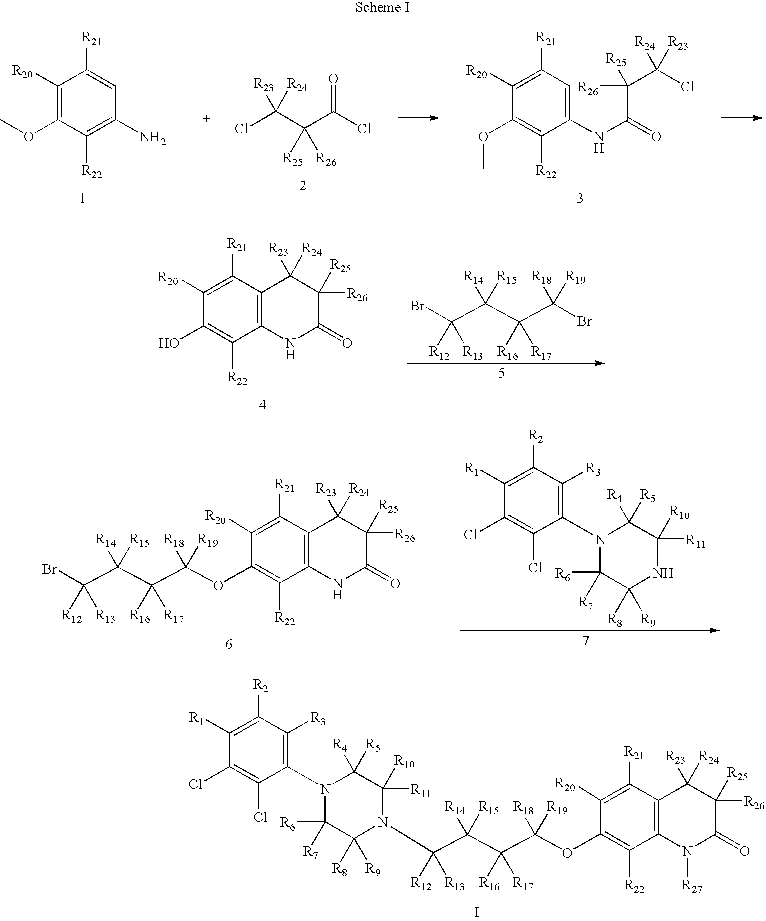

Compound 1 is reacted with compound 2 in the presence of an appropriate base, such as sodium bicarbonate, in an appropriate solvent, such as toluene, to give compound 3. Compound 3 is reacted with an appropriate Lewis acid, such as aluminum chloride, at elevated temperature, in an appropriate solvent, such as N,N-dimethylacetamide, to give compound 4. Compound 4 is reacted with compound 5 in the presence of an appropriate base, such as potassium carbonate, in an appropriate solvent, such as N,N-dimethylformamide, to give compound 6. Compound 6 is reacted with compound 7 in the presence of an appropriate base, such as triethylamine, in an appropriate solvent, such as acetonitrile, to give a compound of Formula I.

Deuterium can be incorporated to different positions synthetically, according to the synthetic procedures as shown in Scheme I, by using appropriate deuterated intermediates. For example, to introduce deuterium at one or more positions of R₂₀-R₂₂, compound 1 with the corresponding deuterium substitutions can be used. To introduce deuterium at one or more positions of R₂₃-R₂₆, compound 2 with the corresponding deuterium substitutions can be used. To introduce deuterium at one or more positions of R₁₂-R₁₉, compound 5 with the corresponding deuterium substitutions can be used. To introduce deuterium at one or more positions of R₁-R₁₁, compound 7 with the corresponding deuterium substitutions can be used.

Deuterium can also be incorporated to various positions having an exchangeable proton, such as the dihydroquinolone N—H, via proton-deuterium equilibrium exchange. For example, to introduce deuterium at R₂₇, this proton may be replaced with deuterium selectively or non-selectively through a proton-deuterium exchange method known in the art.

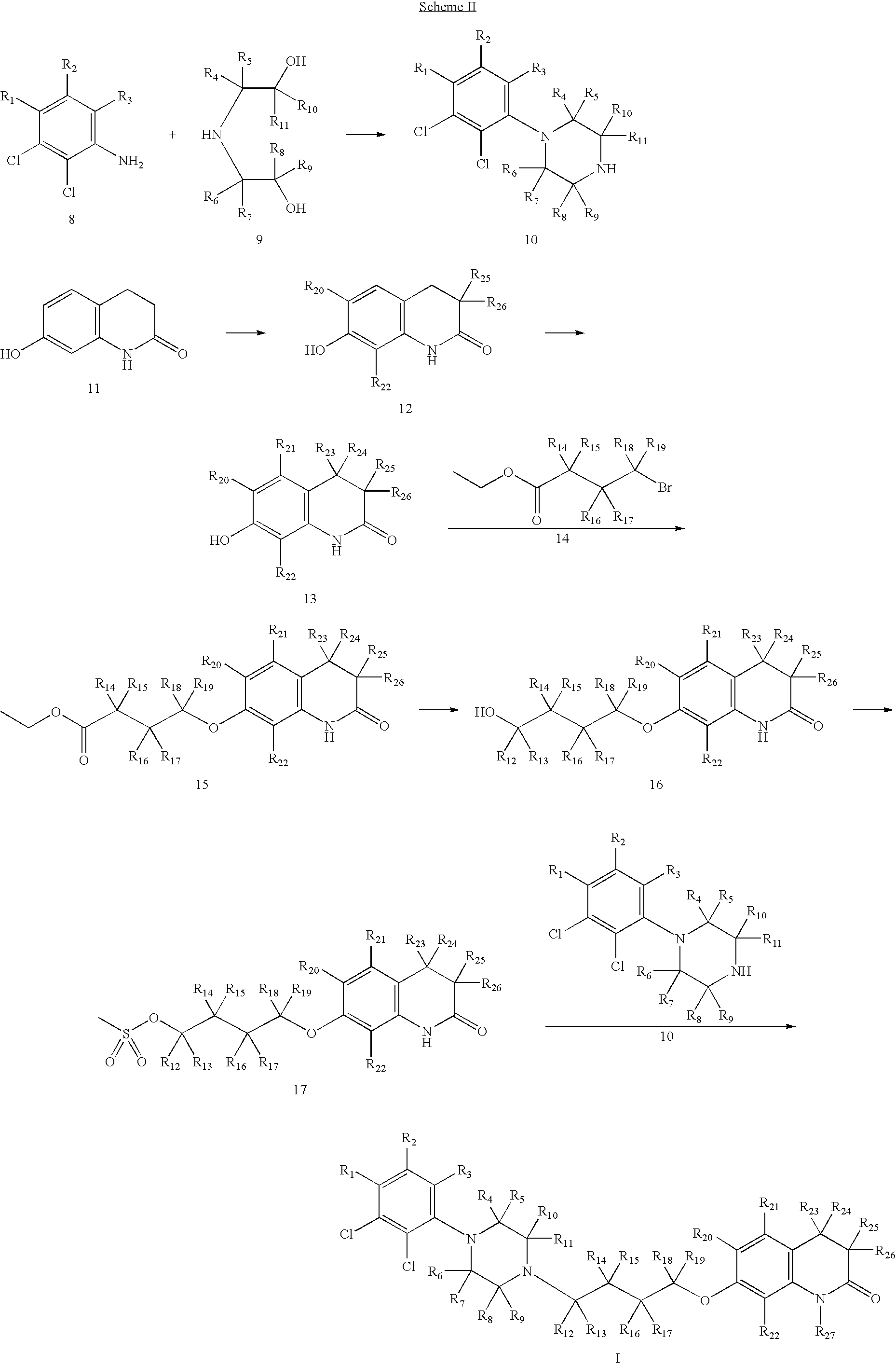

Compound 8 is reacted with compound 9 in the presence of an appropriate dehydrating agent, such as a mixture of phosphorus pentoxide and triethylamine hydrochloride, at elevated temperature, to give compound 10. Compound 11 is reacted with an appropriate base, such as potassium carbonate, at elevated temperature, in an appropriate solvent, such as deuterium oxide, to give compound 12. Compound 12 is reacted with an appropriate deuterium source, such as deuterium oxide, in the presence of an appropriate catalyst, such as a mixture of hydrogen gas and palladium on carbon, to give compound 13. Compound 13 is reacted with compound 14 in the presence of an appropriate base, such as sodium hydride, in an appropriate solvent, such as N,N-dimethylformamide, to give compound 15. Compound 15 is reacted with an reducing agent, such as lithium aluminum hydride, in an appropriate solvent, such as a tetrahydrofuran, to give compound 16. Compound 16 is reacted with an appropriate activating agent, such as methanesulfonyl chloride, in the presence of an appropriate base, such as triethylamine, in an appropriate solvent, such as dichloromethane, to give compound 17. Compound 17 is reacted with compound 10, in the presence of an appropriate base, such as triethylamine, in an appropriate solvent, such as acetonitrile, to give a compound of Formula I.

Deuterium can be incorporated to different positions synthetically, according to the synthetic procedures as shown in Scheme I, by using appropriate deuterated intermediates. For example, to introduce deuterium at one or more positions of R₂₀, R₂₂, and R₂₅-R₂₆, deuterium oxide can be used. To introduce deuterium at one or more positions of R₂₁, and R₂₃-R₂₄, deuterium oxide can be used. To introduce deuterium at one or more positions of R₁₄-R₁₉, compound 14 with the corresponding deuterium substitutions can be used. To introduce deuterium at one or more positions of R₁-R₃, compound 8 with the corresponding deuterium substitutions can be used. To introduce deuterium at one or more positions of R₄-R₁₁, compound 9 with the corresponding deuterium substitutions can be used. To introduce deuterium at one or more positions of R₁₂-R₁₃, lithium aluminum deuteride can be used. Deuterium can also be incorporated to various positions having an exchangeable proton, such as the dihydroquinolone N—H, via proton-deuterium equilibrium exchange. For example, to introduce deuterium at R₂₇, this proton may be replaced with deuterium selectively or non-selectively through a proton-deuterium exchange method known in the art.

### EXAMPLE 1

### 7-[4-(4-(2,3-Dichlorophenyl)piperazin-1-yl)butoxy]-3,4-dihydro-2(1H)-quinolinone

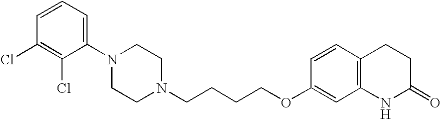

### Step 1

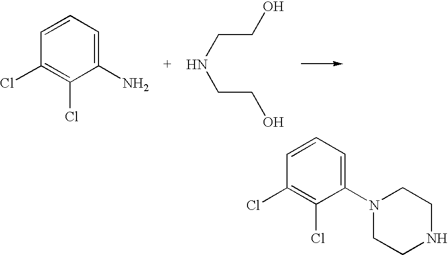

1-(2,3-Dichlorophenyl)piperazine: A mixture of phosphorus pentoxide (17.52 g, 123.43 mmol) and triethylamine hydrochloride (16.99 g, 123.43 mmol) was melted at 220° C. under argon with vigorous stirring. To this, 2,3-dichloroaniline (5.00 g, 30.86 mmol) and diethanolamine (3.24 g, 30.82 mmol) were added sequentially at the same temperature and heating continued for 4 h. The reaction mixture was cooled to 150° C. and quenched by the careful addition of boiling water (150 mL). The obtained tarry product was partitioned between ethyl acetate and water. The aqueous layer was cooled to 0° C. and basified to pH 10 with 10% sodium hydroxide. Standard extractive work up provided a crude residue which was purified by chromatography on neutral alumina (3% methanol in chloroform) to give the title compound as a brown oil (1.10 g, 15%). ¹H NMR (400 MHz, CDCl₃) δ 2.94-3.11 (m, 8H), 6.95 (dd, J=6.1, 3.5 Hz, 1H), 7.11-7.18 (m, 2H); IR (film) υ 3055, 2983, 2828, 1578, 1444, 1265 cm⁻¹; MS 231, 233 [(M+1), (M+3)].

### Step 2

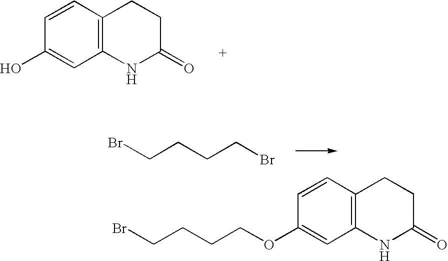

7-(4-Bromobutoxy)-3,4-dihydro-2(1H)-quinolinone: Potassium hydroxide (0.120 g, 2.139 mmol) was added to a solution of 7-hydroxy-3,4-dihydro-2(1H)-quinolinone (0.200 g, 1.226 mmol) in 2-propanol (3 mL). After a clear solution was obtained, 1,4-dibromobutane (0.44 mL, 3.685 mmol) was added and the mixture was refluxed for 16 h. It was then cooled, diluted with ethyl acetate and filtered. The filtrate was concentrated to provide a crude residue which was purified by chromatography on neutral alumina (20% acetone in dichloromethane) to give the title compound as a white solid (0.200 g, 55%). ¹H NMR (400 MHz, CDCl₃) δ 1.87-1.98 (m, 2H), 2.00-2.10 (m, 2H), 2.62 (t, J=7.7 Hz, 2H), 2.90 (t, J=7.5 Hz, 2H), 3.49 (t, J=6.8 Hz, 2H), 3.97 (t, J=6.0 Hz, 2H), 6.29 (d, J=2.1 Hz, 1H), 6.52 (dd, J=8.3, 2.5 Hz, 1H), 7.05 (d, J=8.3 Hz, 1H), 7.69 (br, exchangeable with D₂O, 1H); IR (KBr) υ 2928, 1677, 1631, 1594, 1383 cm⁻¹; MS 298, 300 [(M+1), (M+3)].

### Step 3

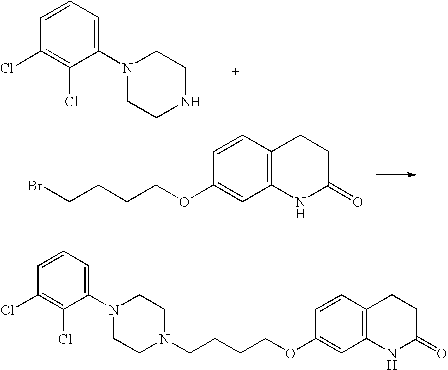

7-[4-(4-(2,3-Dichlorophenyl)piperazin-1-yl)butoxy]-3,4-dihydro-2(1H)-quinolinone: A mixture of 1-(2,3-dichlorophenyl)piperazine (0.155 g, 0.671 mmol), 7-(4-bromobutoxy)-3,4-dihydro-2(1H)-quinolinone (0.200 g, 0.671 mmol), triethylamine (0.14 mL, 1.00 mmol) and acetonitrile (5 mL) was refluxed for 4 h. The volatiles were distilled off under reduced pressure and the residue was purified by silica gel column chromatography (3% methanol in dichloromethane) to give the title compound as an off-white solid (0.160 g, 53%). m.p. 127-130° C.; ¹H NMR (400 MHz, pyridine-d₅) δ 1.62-1.71 (m, 2H), 1.79-1.88 (m, 2H), 2.37 (t, J=7.3 Hz, 2H), 2.50-2.60 (m, 4H), 2.64 (t, J=7.4 Hz, 2H), 2.82 (t, J=7.4 Hz, 2H), 2.96-3.06 (m, 4H), 4.04 (t, J=6.5 Hz, 2H), 6.7 4 (dd, J=8.1, 2.5 Hz, 1H), 6.85 (d, J=2.5 Hz, 1H), 6.97 (d, J=8.0 Hz, 1H), 7.10-7.26 (m, 3H), 11.16 (br, exchangeable with D₂O, 1H); IR (KBr) υ 3447, 2943, 2822, 1681, 1628, 1587, 1451, 1375 cm⁻¹; MS 448, 450 [(M+1), (M+3)].

### EXAMPLE 2

### 7-[4-(4-(2,3-Dichlorophenyl)piperazin-1-yl)butoxy]-3,4-dihydro-2(1H)-quinolinone-3,3,4,4,6,8-d₆

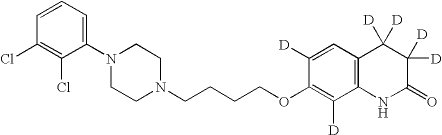

### Step 1

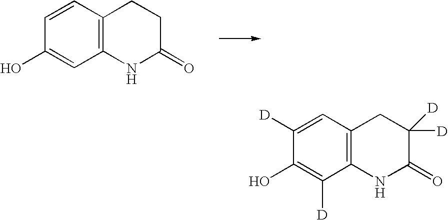

7-Hydroxy-3,4-dihydro-2(1H)-quinolinone-3,3,6,8-d₄: A mixture of 7-hydroxy-3,4-dihydro-2(1H)-quinolinone (4.00 g, 24.51 mmol), anhydrous potassium carbonate (6.78 g, 49.06 mmol) and D₂O (40 mL) was heated at 100-105° C. for 16 h. The reaction mixture was cooled to room temperature and extracted with ethyl acetate. The organic layer was dried over Na₂SO₄ and concentrated under reduced pressure. To the residue, anhydrous potassium carbonate (5.08 g, 36.76 mmol) and D₂O (25 mL) were added and the mixture was heated at 100-105° C. for 5 h. The reaction mixture was worked up as before to give the title compound as a white solid (2.70 g, 66%; deuteration 98%). ¹H NMR (400 MHz, DMSO-d₆) δ 2.71 (s, 2H), 6.90 (s, 1H), 9.88 (br, 1H); IR (KBr) υ 2438, 2330, 1650, 1609, 1469, 1381, 1233 cm⁻¹; MS 168 (M+1).

### Step 2

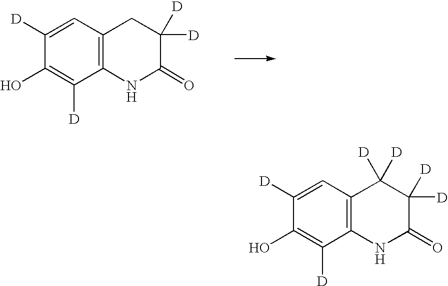

7-Hydroxy-3,4-dihydro-2(1H)-quinolinone-3,3,4,4,6,8-d₆: A mixture of 7-hydroxy-3,4-dihydro-2(1H)-quinolinone-3,3,6,8-d₄ (0.700 g, 4.19 mmol), 10% palladium on carbon (0.070 g) and D₂O (15 mL) was heated in a steel bomb at 100° C. under an atmosphere of hydrogen for 3 h. The reaction mixture was filtered through a pad of Celite and the filtrate was extracted with ethyl acetate. The organic layer was dried over Na₂SO₄ and concentrated under reduced pressure. To the residue, 10% palladium on carbon (0.070 g) and D₂O (15 mL) were added and the mixture was heated in a steel bomb at 100° C. under an atmosphere of hydrogen for 2 h. The reaction mixture was worked up as before to give the title compound as a white solid (0.600 g, 85%; deuteration 98%). ¹H NMR (400 MHz, CD₃OD, stoichiometric quantity of 4-nitroanisole) δ 6.95 (s, 1H); IR (KBr) υ 3268, 2441, 1651, 1603, 1466, 1371, 1313, 1237 cm⁻¹; MS 170 (M+1).

### Step 3

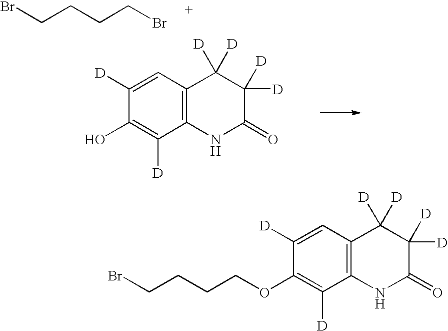

7-(4-Bromobutoxy)-3,4-dihydro-2(1H)-quinolinone-3,3,4,4,6,8-d₆: Following a similar procedure to that described in Example 1 step 2, sodium hydride (60% in mineral oil, 0.024 g, 0.600 mmol), 7-hydroxy-3,4-dihydro-2(1H)-quinolinone-3,3,4,4,6,8-d₆ (0.100 g, 0.591 mmol), 1,4-dibromobutane (0.14 mL, 1.172 mmol), and dry DMF (1 mL) were reacted to give the title compound as a white solid (0.095 g, 53%). ¹HNMR(400 MHz, CDCl₃) δ 1.87-1.97 (m, 2H), 2.01-2.10 (m, 2H), 3.49 (t, J=6.6 Hz, 2H), 3.96 (t, J=6.1 Hz, 2H), 6.52 (d, J=8.4 Hz, 0.35H), 7.05 (d, J=8.0 Hz, 1H), 7.78 (br, exchangeable with D₂O, 1H); IR (KBr) υ 3200, 3091, 2969, 2927, 2873, 1675, 1622, 1580, 1471, 1369, 1304, 1229 cm⁻¹; MS 304, 306 [(M+1), (M+3)].

### Step 4

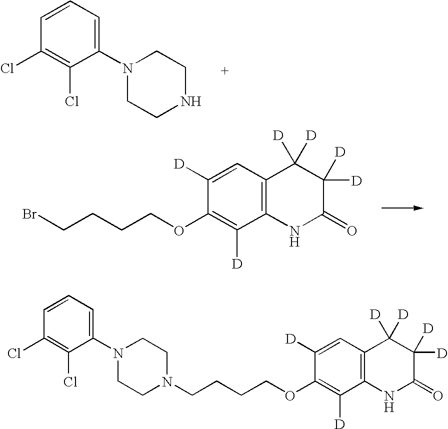

7-[4-(4-(2,3-Dichlorophenyl)piperazin-1-yl)butoxy]-3,4-dihydro-2(1H)-quinolinone-3,3,4,4,6,8-d₆: Following a similar procedure to that described in Example 1 step 3, 1-(2,3-dichlorophenyl)piperazine (0.061 g, 0.264 mmol), 7-(4-bromobutoxy)-3,4-dihydro-2(1H)-quinolinone-3,3,4,4,6,8-d₆ (0.080 g, 0.263 mmol), triethylamine (0.07 mL, 0.502 mmol) and acetonitrile (1.5 mL) were reacted to give the title compound as an off-white solid (0.075 g, 63%). m.p. 126-128° C.; ¹H NMR (400 MHz, pyridine-d₅) δ 1.61-1.70 (m, 2H), 1.78-1.87 (m, 2H), 2.37 (t, J=7.1 Hz, 2H), 2.48-2.61 (m, 4H), 2.96-3.07 (m, 4H), 4.04 (t, J=6.4 Hz, 2H), 6.75 (d, J=8.1 Hz, 0.33H); 6.97 (d, J=8.1 Hz, 1H), 7.11-7.25 (m, 3H), 11.18 (br, exchangeable with D₂O, 1H); IR (KBr) υ 2946, 2819, 1679, 1620, 1579, 1457, 1360, 1241 cm⁻¹; MS 454, 455, 456 [(M+1), (M+2), (M+3)].

### EXAMPLE 3

### 7-[4-(4-(2,3-Dichlorophenyl)piperazin-1-yl)butoxy-4,4-d₂]-3,4-dihydro-2(1H)-quinolinone-3,3,4,4,6,8-d₆

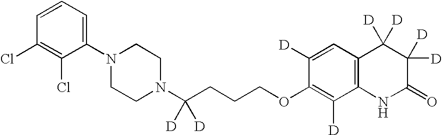

### Step 1

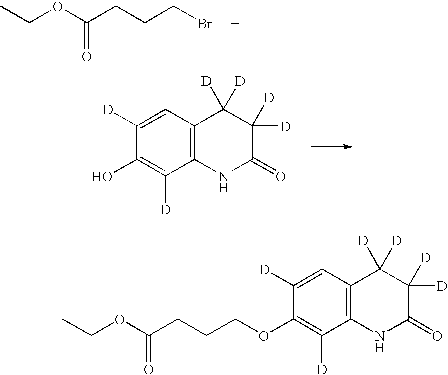

Ethyl 4-(2-oxo-1,2,3,4-tetrahydroquinolin-7-yloxy-3,3,4,4,6,8-d₆)butanoate: Sodium hydride (60% in mineral oil, 0.078 g, 1.95 mmol) was added to a solution 7-hydroxy-3,4-dihydro-2(1H)-quinolinone-3,3,4,4,6,8-d₆ (0.300 g, 1.77 mmol) in dry DMF (2 mL). After 10 min, ethyl 4-bromobutyrate (0.38 mL, 2.66 mmol) was added and the reaction mixture was stirred at 100° C. for 1 h. It was cooled to 0° C. and D₂O (0.5 mL) was added dropwise to destroy excess sodium hydride. Standard extractive work up provided a crude residue which was purified by chromatography on neutral alumina (20-25% acetone in dichloromethane) to give the title compound as a white solid (0.340 g, 68%). ¹H NMR (400 MHz, CDCl₃) δ 1.26 (t, J=7.3 Hz, 3H), 2.03-2.13 (m, 2H), 2.49 (t, J=7.3 Hz, 2H), 3.97 (t, J=6.0 Hz, 2H), 4.14 (q, J=7.2 Hz, 2H), 7.03 (s, 1H), 7.85 (br, exchangeable with D₂O, 1H); IR (KBr) υ 2972, 1724, 1686, 1619, 1580, 1472, 1359 cm⁻¹; MS 284 (M+1).

### Step 2

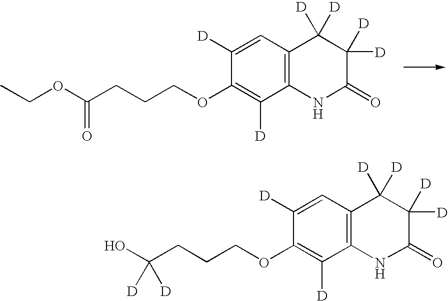

7-(4-Hydroxybutoxy-4,4-d₂)-3,4-dihydro-2(1H)-quinolinone-3,3,4,4,6,8-d₆: A solution of ethyl 4-(2-oxo-1,2,3,4-tetrahydroquinolin-7-yloxy-3,3,4,4,6,8-d₆)butanoate (0.300 g, 1.059 mmol) in dry THF (3 mL) was added dropwise to a stirred suspension of lithium aluminum deuteride (0.058 g, 1.382 mmol) in dry THF (2 mL) at −10° C. The mixture was stirred at −10° C. for 1 h, after which D₂O (0.5 mL) was added cautiously. The formed precipitate was filtered and washed with ethyl acetate. The filtrate and washings were combined, washed with 6N DCl (2 mL) and concentrated in vacuo. The obtained residue was triturated with hexane to give the title compound as a solid (0.160 g, 62%). ¹H NMR (400 MHz, CDCl₃) δ 1.68-1.77 (m, 2H), 1.80-92 (m, 2H), 3.98 (t, J=6.2 Hz, 2H), 7.05 (s, 1H), 7.95 (br, 1H); IR (KBr) υ3393, 2945, 2515, 1654, 1571, 1453, 1376 cm⁻¹; MS 244 (M+1).

### Step 3

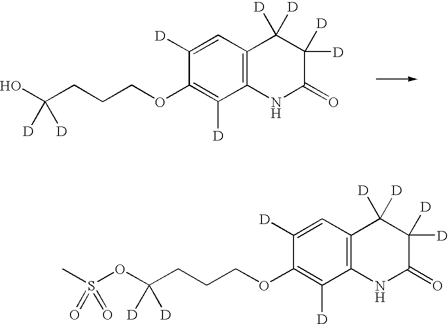

4-(2-Oxo-1,2,3,4-tetrahydroquinolin-7-yloxy-3,3,4,4,6,8-d₆)butyl-1,1-d₂ methanesulfonate: Methanesulfonyl chloride (0.07 mL, 0.904 mmol) was added dropwise to a mixture of 7-(4-hydroxybutoxy-4,4-d₂)-3,4-dihydro-2(1H)-quinolinone-3,3,4,4,6,8-d₆ (0.150 g, 0.616 mmol), triethylamine (0.26 mL, 1.865 mmol) and dichloromethane (2 mL) at 0° C. The reaction mixture was stirred for 0.5 h at the same temperature. Standard extractive work up provided a crude residue which was purified by chromatography on neutral alumina (50% acetone in dichloromethane) to give the title compound as a white solid (0.145 g, 73%). ¹H NMR (400 MHz, CDCl₃) δ 1.84-1.99 (m, 4H), 3.03 (s, 3H), 3.98 (t, J=5.6 Hz, 2H), 7.05 (s, 1H), 7.85 (br, exchangeable with D₂O, 1H); IR (KBr)υ 3199, 3093, 2963, 1675, 1619, 1579, 1470, 1353 cm⁻¹; MS 322 (M+1).

### Step 4

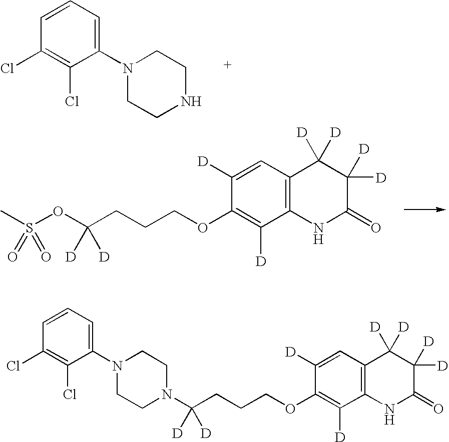

7-[4-(4-(2,3-Dichlorophenyl)piperazin-1-yl)butoxy-4,4-d₂]-3,4-dihydro-2(1H)-quinolinone-3,3,4,4,6,8-d₆: A mixture of 1-(2,3-dichlorophenyl)piperazine (0.144 g, 0.623 mmol), 4-(2-oxo-1,2,3,4-tetrahydroquinolin-7-yloxy-3,3,4,4,6,8-d₆)butyl-1,1-d₂ methanesulfonate (0.200 g, 0.622 mmol), triethylamine (0.22 mL, 1.578 mmol) and acetonitrile (2 mL) was refluxed for 4 h. The volatiles were distilled off under reduced pressure and the residue was purified by silica gel column chromatography (2-4% methanol in dichloromethane) to give the title compound as a brownish solid (0.220 g, 77%). m.p. 130-134° C.; ¹H NMR (400 MHz, pyridine-d₅) δ 1.61-1.69 (m, 2H), 1.78-1.88 (m, 2H), 2.46-2.61 (m, 4H), 2.94-3.06 (m, 4H), 4.04 (t, J=6.4 Hz, 2H), 6.74 (d, J=8.2 Hz, 0.2H), 6.97 (dd, J=8.0, 1.4 Hz, 1H), 7.12-7.25 (m, 3H), 11.13 (br, exchangeable with D₂O, 1H); IR (KBr) υ 2946, 1679, 1620, 1578, 1456, 1355, 1298, 1240 cm⁻¹; MS 456, 457, 458 [(M+1), (M+2), (M+3)].

### EXAMPLE 4

### 7-[4-(4-(2,3-Dichlorophenyl)piperazin-1-yl)butoxy-d₈]-3,4-dihydro-2(1H)-quinolinone-3,3,4,4,6,8-d₆

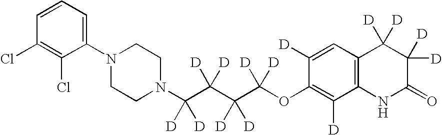

### Step 1

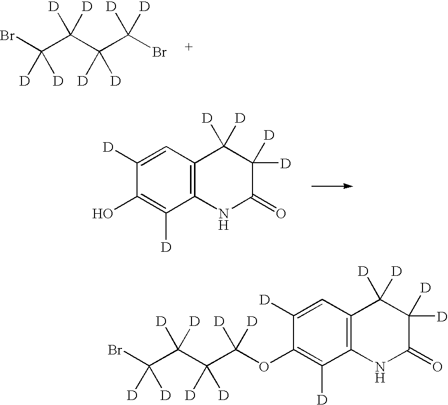

7-(4-Bromobutoxy-d₈)-3,4-dihydro-2(1H)-quinolinone-3,3,4,4,6,8-d₆: Following a similar procedure to that described in Example 1 step 2, sodium hydride (60% in mineral oil, 0.048 g, 1.200 mmol), 7-hydroxy-3,4-dihydro-2(1H)-quinolinone-3,3,4,4,6,8-d₆ (0.200 g, 1.182 mmol), 1,4-dibromobutane-d₈ (0.28 mL, 2.343 mmol), and dry DMF (2 mL), were reacted to give the title compound as a white solid (0.200 g, 54%). ¹H NMR (400 MHz, CDCl₃, stoichiometric quantity of 4-nitroanisole) δ 7.05 (s, 1H), 8.07 (br, exchangeable with D₂O, 1H); IR (KBr) υ 3198, 3091, 2969, 2877, 2206, 2091, 1676, 1624, 1578, 1470, 1373, 1304, 1229 cm⁻¹; MS 312, 314 [(M+1), (M+3)].

### Step 2

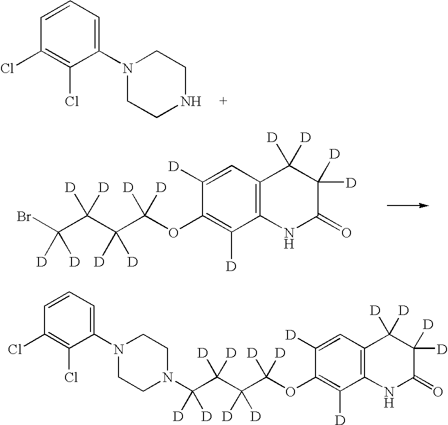

7-[4-(4-(2,3-Dichlorophenyl)piperazin-1-yl)butoxy-d₈]-3,4-dihydro-2(1H)-quinolinone-3,3,4,4,6,8-d₆: Following a similar procedure to that described in Example 1 step 3, 1-(2,3-dichlorophenyl)piperazine (0.074 g, 0.320 mmol), 7-(4-bromobutoxy-d₈)-3,4-dihydro-2(1H)-quinolinone-3,3,4,4,6,8-d₆ (0.100 g, 0.320 mmol), triethylamine (0.09 mL, 0.646 mmol), and acetonitrile (2 mL), were reacted to give the title compound as an off-white solid (0.105 g, 71%). m.p. 129-133° C.; ¹H NMR (400 MHz, pyridine-d₅) δ 2.34-2.51 (m, 4H), 2.83-2.95 (m, 4H), 6.63 (d, J=8.2 Hz, 0.2H), 6.86 (dd, J=8.1, 1.5 Hz, 1H), 7.00-7.14 (m, 3H), 11.00 (br, exchangeable with D₂O, 1H); IR (KBr) υ 1679, 1621, 1578, 1455, 1359, 1296, 1241 cm⁻¹; MS 462, 463, 464 [(M+1), (M+2), (M+3)].

### EXAMPLE 5

### 7-[4-(4-(2,3-Dichlorophenyl)piperazin-1-yl)butoxy-d₈]-3,4-dihydro-2(1H)-quinolinone

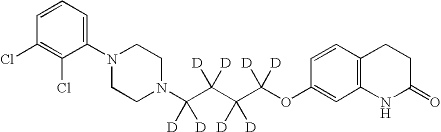

### Step 1

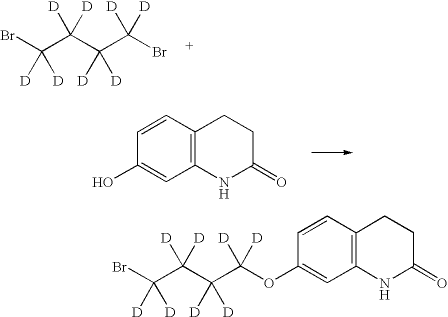

7-(4-Bromobutoxy-d₈)-3,4-dihydro-2(1H)-quinolinone: Following a similar procedure to that described in Example 1 step 2, potassium hydroxide (0.215 g, 3.832 mmol), 7-hydroxy-3,4-dihydro-2(1H)-quinolinone (0.500 g, 3.064 mmol), 1,4-dibromobutane-d₈ (1.10 mL, 9.204 mmol), and 2-propanol (7 mL), were reacted to provide a crude residue which was purified by chromatography on neutral alumina (10-15% acetone in dichloromethane) to give the title compound as an off-white solid (0.600 g, 64%). ¹H NMR (400 MHz, CDCl₃) δ 2.62 (t, J=7.5 Hz, 2H), 2.90 (t, J=7.5 Hz, 2H), 6.33 (d, J=2.3 Hz, 1H), 6.51 (dd, J=8.2, 2.3 Hz, 1H), 7.05 (d, J=8.2 Hz, 1H), 8.27 (br, exchangeable with D₂O, 1H); IR (KBr) υ 2903, 2208, 2091, 1678, 1629, 1590, 1522, 1487, 1384, 1306, 1272 cm⁻¹; MS 306, 308 [(M+1), (M+3)].

### Step 2

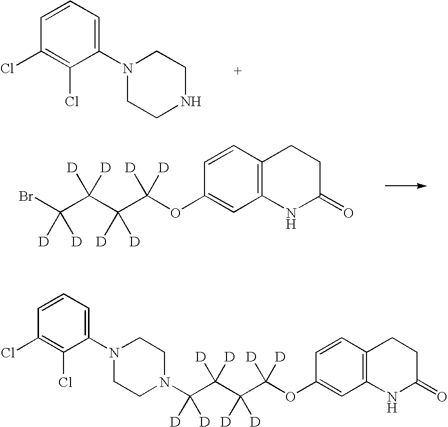

7-[4-(4-(2,3-Dichlorophenyl)piperazin-1-yl)butoxy-d₈]-3,4-dihydro-2(1H)-quinolinone: Following a similar procedure to that described in Example 1 step 3, 1-(2,3-dichlorophenyl)piperazine (0.227 g, 0.982 mmol), 7-(4-bromobutoxy-d₈)-3,4-dihydro-2(1H)-quinolinone (0.300 g, 0.980 mmol), triethylamine (0.27 mL, 1.937 mmol) and acetonitrile (3 mL), were reacted to give the title compound as an off-white solid (0.300 g, 67%). m.p. 130-132° C.; ¹H NMR (400 MHz, pyridine-d₅) δ 2.50-2.59 (m, 4H), 2.64 (t, J=7.4 Hz, 2H), 2.82 (t, J=7.4 Hz, 2H), 2.97-3.05 (m, 4H), 6.74 (dd, J=8.1, 2.5 Hz, 1H), 6.85 (d, J=2.5 Hz, 1H), 6.97 (d, J=8.1 Hz, 1H), 7.11-7.25 (m, 3H), 11.15 (br, exchangeable with D₂O, 1H); IR (KBr) υ 2826, 1682, 1625, 1588, 1519, 1449, 1376 cm⁻¹; MS 456, 458 [(M+1), (M+3)].

The invention is further illustrated by the following examples. All IUPAC names were generated using CambridgeSoft's ChemDraw 10.0.

The following compounds can generally be made using the methods described above. It is expected that these compounds when made will have activity similar to those described in the examples above.

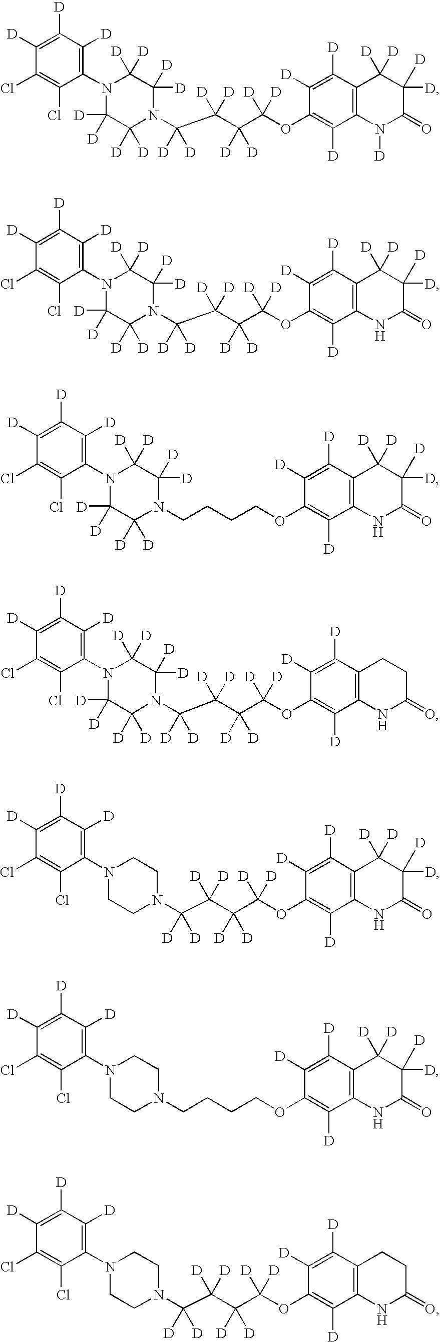

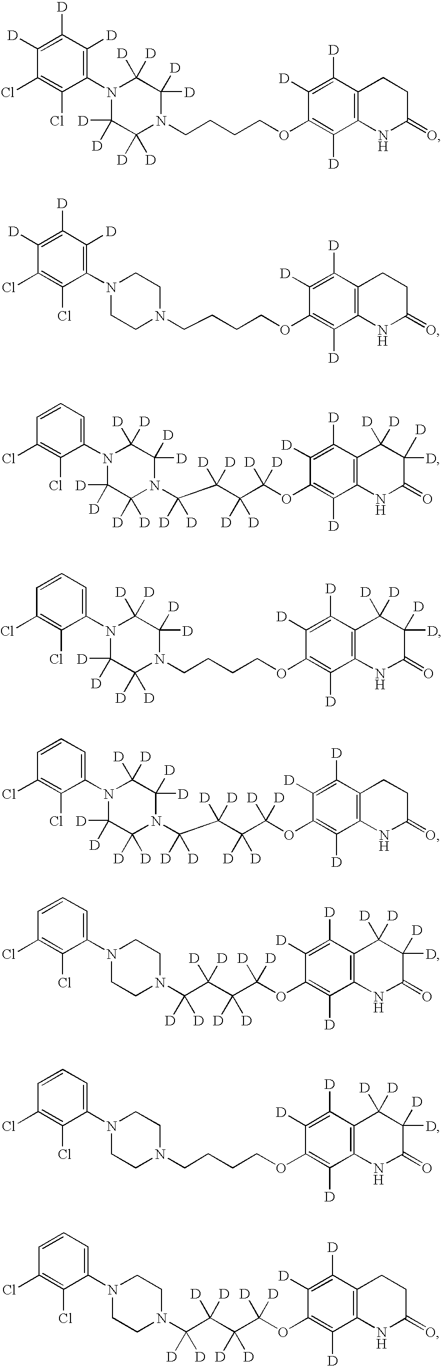

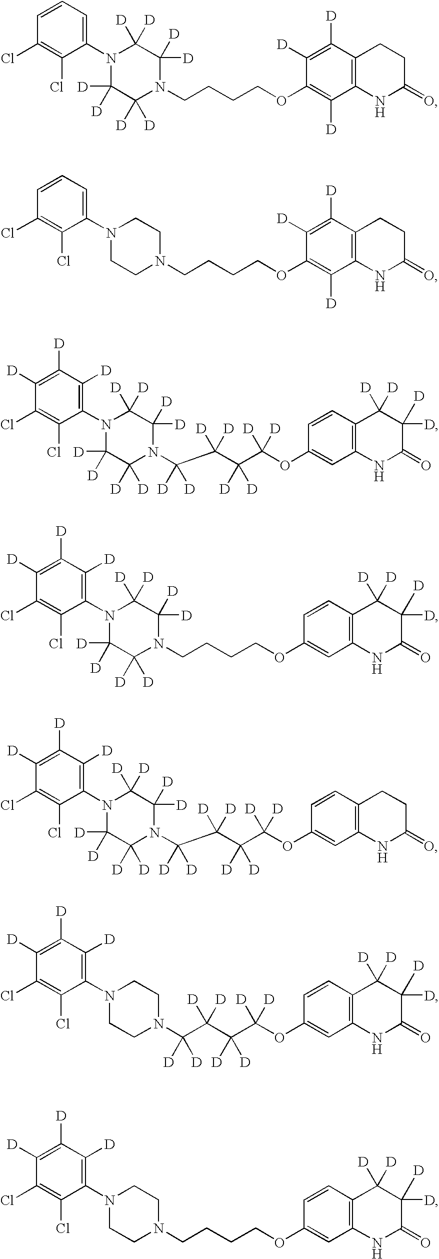

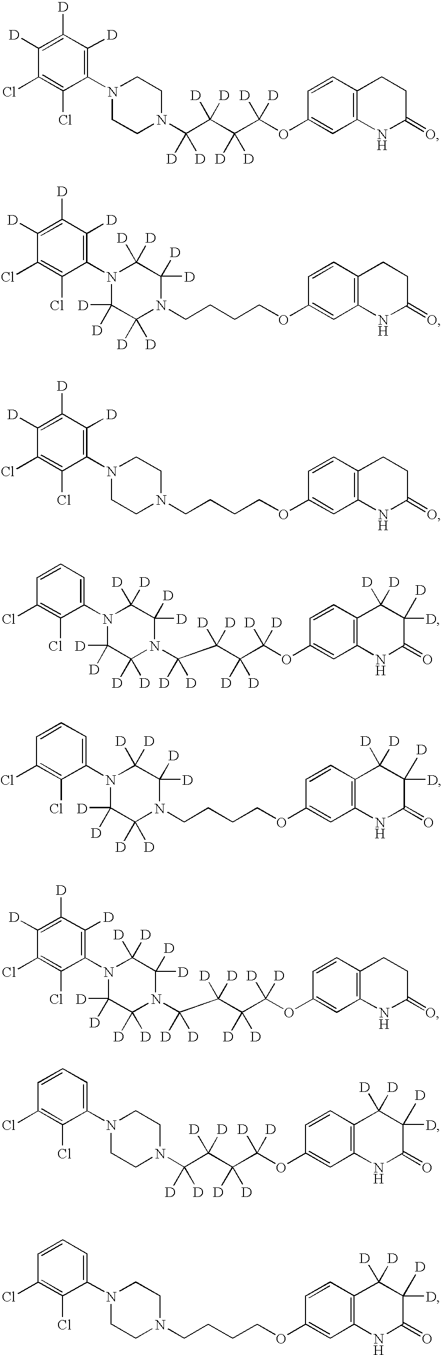

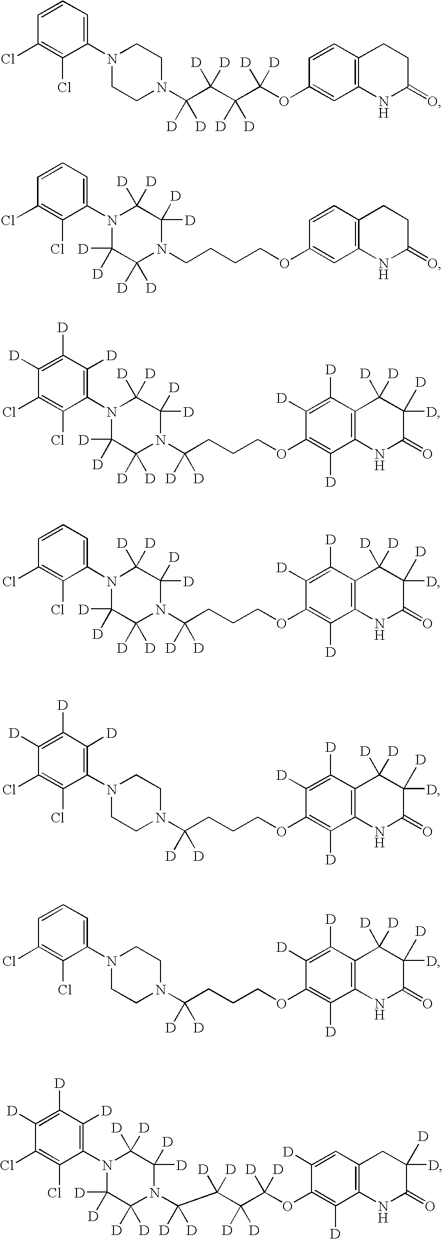

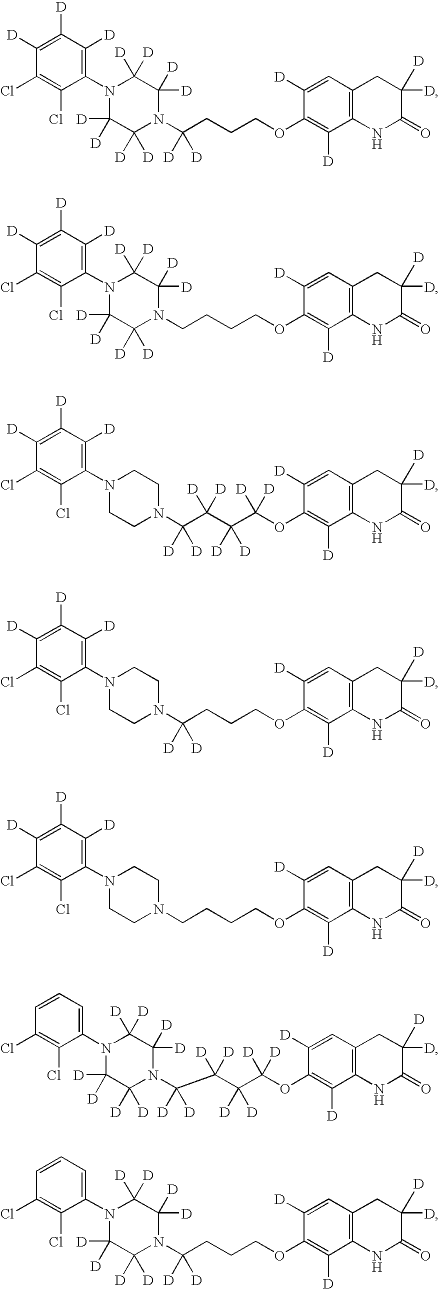

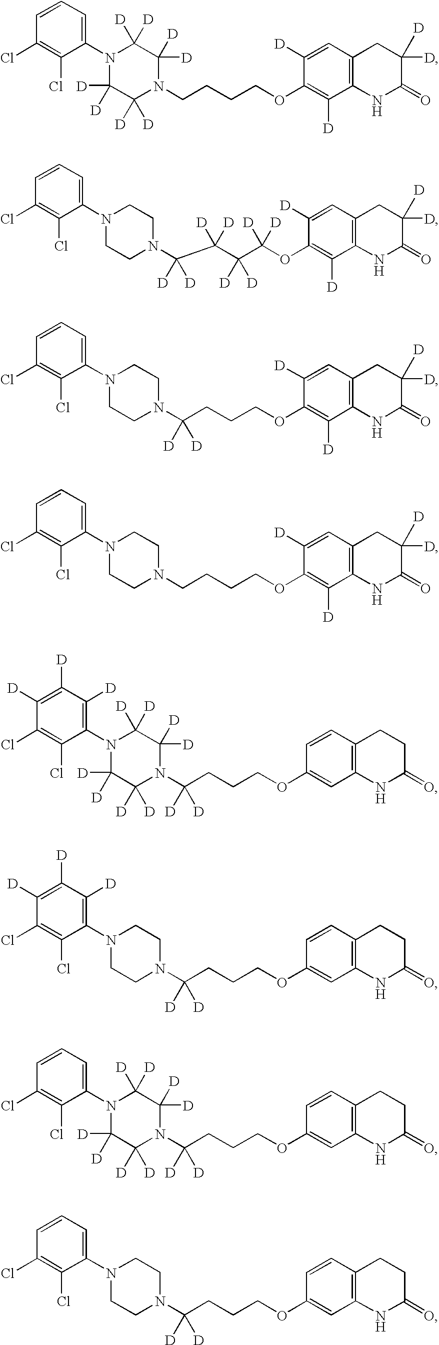

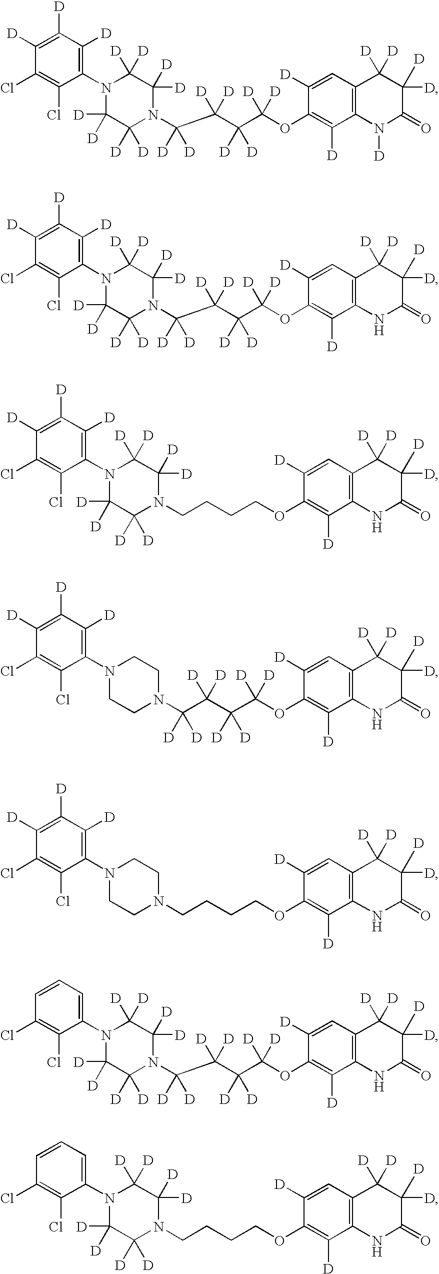

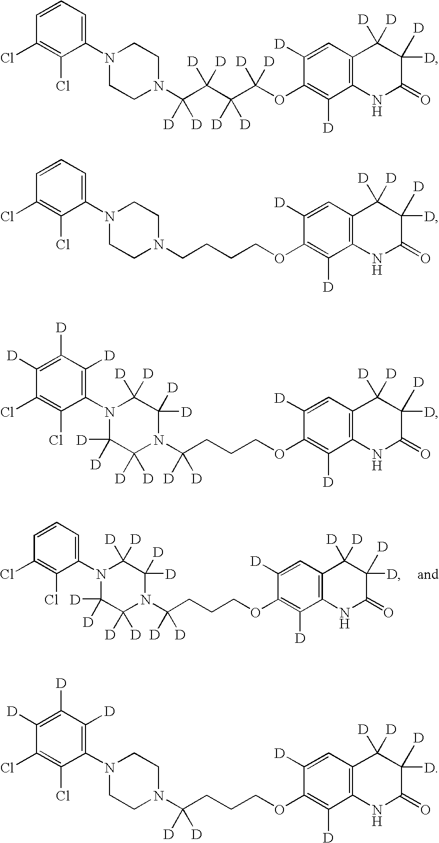

Changes in the metabolic properties of the compounds disclosed herein as compared to their non-isotopically enriched analogs can be shown using the following assays. Compounds listed above which have not yet been made and/or tested are predicted to have changed metabolic properties as shown by one or more of these assays as well.

### Biological Activity Assays

### In Vitro Liver Microsomal Stability Assay

Liver microsomal stability assays are conducted at 1 mg per mL liver microsome protein with an NADPH-generating system in 2% NaHCO₃ (2.2 mM NADPH, 25.6 mM glucose 6-phosphate, 6 units per mL glucose 6-phosphate dehydrogenase and 3.3 mM MgCl₂). Test compounds are prepared as solutions in 20% acetonitrile-water and added to the assay mixture (final assay concentration 1 μM) and incubated at 37° C. Final concentration of acetonitrile in the assay should be <1%. Aliquots (50 μL) are taken out at times 0, 0.25, 0.30, and 1 hours, and diluted with ice cold acetonitrile (200 μL) to stop the reactions. Samples are centrifuged at 12,000 RPM for 10 min to precipitate proteins. Supernatants are transferred to micro centrifuge tubes and stored for LC/MS/MS analysis of the degradation half-life of the test compounds. It has thus been found that the compounds of formula (I) that have been tested in this assay showed improved degradation half-life, as compared to the non-isotopically enriched drug. Some of the compounds showed a decrease of degradation half-life, as compared to the non-isotopically enriched drug. Additionally some of the compounds showed at least 17% increase of degradation half-life, as compared to the non-isotopically enriched drug. Additionally some of the compounds showed at least 18% increase of degradation half-life, as compared to the non-isotopically enriched drug. The degradation half-lives of Examples 2 through 5 are shown in Table 1.

TABLE 1
Results of in vitro human liver microsomal
(HLM) stability assay

% increase of HLM degradation half-life

| | | | |
|---|---|---|---|
| −25%-0% | 0%-50% | 50%-150% | >150% |

| | |
|---|---|
| Example 2 | + |
| Example 3 | + |
| Example 4 | + |
| Example 5 | + |

### In Vitro Metabolism Using Human Cytochrome P₄₅₀ Enzymes

The cytochrome P₄₅₀ enzymes are expressed from the corresponding human cDNA using a baculovirus expression system (BD Biosciences, San Jose, Calif.). A 0.25 milliliter reaction mixture containing 0.8 milligrams per milliliter protein, 1.3 millimolar NADP⁺, 3.3 millimolar glucose-6-phosphate, 0.4 U/mL glucose-6-phosphate dehydrogenase, 3.3 millimolar magnesium chloride and 0.2 millimolar of a compound of Formula I, the corresponding non-isotopically enriched compound or standard or control in 100 millimolar potassium phosphate (pH 7.4) is incubated at 37° C. for 20 min. After incubation, the reaction is stopped by the addition of an appropriate solvent (e.g., acetonitrile, 20% trichloroacetic acid, 94% acetonitrile/6% glacial acetic acid, 70% perchloric acid, 94% acetonitrile/6% glacial acetic acid) and centrifuged (10,000 g) for 3 min. The supernatant is analyzed by HPLC/MS/MS.

| Cytochrome P₄₅₀ | Standard |
|---|---|
| CYP1A2 | Phenacetin |
| CYP2A6 | Coumarin |
| CYP2B6 | [¹³C]-(S)-mephenytoin |
| CYP2C8 | Paclitaxel |
| CYP2C9 | Diclofenac |
| CYP2C19 | [¹³C]-(S)-mephenytoin |
| CYP2D6 | (+/−)-Bufuralol |
| CYP2E1 | Chlorzoxazone |
| CYP3A4 | Testosterone |
| CYP4A | [¹³C]-Lauric acid |

### CYP3A4

Solution A—NADPH-regenerating system: To a glass tube on ice were added sequentially: Water (8 mL) in NADP⁺ (20 mg), glucose-6-phosphate (20 mg), magnesium chloride hexahydrate (13.3 mg) and glucose-6-phosphate dehydrogenase (8 units).

Solution B: To a glass tube on ice were added sequentially: 0.5 M KH₂PO₄ (pH 7.4, 9.6 mL), water (2.328 mL), CYP3A4 (60 μL of 1 picomol/microliter), and dibenzylfluorescein (2 mM in 100% acetonitrile, 12 microliter).

Solution A was transferred to a 96-well black plate (80 microliter per well), followed by various concentrations of a solution of Compound in 20% acetonitrile-water (20 microliter per well). The reaction was initiated by adding 100 microliter of solution B to each well of the 96-well plate. The plate was incubated for 10 minutes at 37° C. in the dark. The reaction was stopped by adding 75 microliter of stop buffer (2 N NaOH) to each well, and incubated at 37° C. for 2 hours and the end point was measured in a fluorometer plate reader at λₑₓ=485 nm and λₑₘ=538 nm. Ketoconazole was used as positive control (IC₅₀=5.7 nanomolar). The compounds of Examples 1, 2, 3, 4, and 5 showed IC50,s of 5.9 μM, 12.9 μM, >100 μM, 7.8 μM, and 7.7 μM respectively.

### CYP2D6

Solution A—NADPH-regenerating system: To a glass tube on ice were added sequentially: Water (9 mL), 0.5 M KH2PO4 (1 mL) in NADP⁺ (0.136 mg), glucose-6-phosphate (2.72 mg), magnesium chloride hexahydrate (1.81 mg) and glucose-6-phosphate dehydrogenase (8 units).

Solution B: To a glass tube on ice were added sequentially: 0.5 M KH₂PO₄ (pH 7.4, 3 mL), water (6.725 mL), CYP2D6 (175 μL of 4 picomol/microliter), and 3-[2-(N,N diethyl-N-methylamino)ethyl]-7-methoxy-4-methylcoumarin (1 mM in 50 mMKH2PO4 pH 7.4, 100 microliter).

Solution A was transferred to a 96-well black plate (92 microliter per well), followed by various concentrations of a solution of Compound in 25% acetonitrile-water (8 microliter per well). The reaction was initiated by adding 100 microliter of solution B to each well of the 96-well plate. The plate was incubated for 40 minutes at 37° C. in the dark. The reaction was stopped by adding 75 microliter of stop buffer (4:1 acetonitrile-0.5 M Tris base) to each well, and the end point was measured in a fluorometer plate reader at λₑₓ=360 nm and λₑₘ=460 nm. Furafylline was used as positive control (IC₅₀=14.5 nanomolar). The compounds of Examples 1, 2, 3, 4, and 5 showed IC50,s of 7.5 μM, 2.1 μM, 7.1 μM, 5.1 μM, and 1.9 μM respectively.

### Monoamine Oxidase A Inhibition and Oxidative Turnover

The procedure is carried out using the methods described by Weyler, Journal of Biological Chemistry 1985, 260, 13199-13207, which is hereby incorporated by reference in its entirety. Monoamine oxidase A activity is measured spectrophotometrically by monitoring the increase in absorbance at 314 nm on oxidation of kynuramine with formation of 4-hydroxyquinoline. The measurements are carried out, at 30° C., in 50 mM NaPi buffer, pH 7.2, containing 0.2% Triton X-100 (monoamine oxidase assay buffer), plus 1 mM kynuramine, and the desired amount of enzyme in 1 mL total volume.

### Monooamine Oxidase B Inhibition and Oxidative Turnover

The procedure is carried out as described in Uebelhack, Pharmacopsychiatry 1998, 31(5), 187-192, which is hereby incorporated by reference in its entirety.

### Cloned Receptor Affinity Assay

The procedure is carried out as described in Lawler et al., Neuropsychopharmacology 20(6) 612-27 (1999), which is hereby incorporated by reference in its entirety.

### Radioligand Binding Assay

The procedure is carried out as described in Wood et al., Eur. J. Pharmacol. (546) 88-94 (2006), which is hereby incorporated by reference in its entirety.

### Amphetamine-Induced Locomotor Activity

The procedure is carried out as described in Wood et al., Eur. J. Pharmacol. (546) 88-94 (2006), which is hereby incorporated by reference in its entirety.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound of structural Formula I 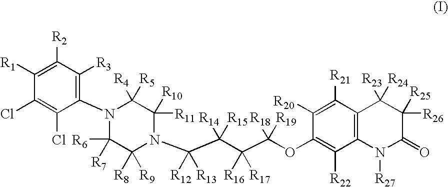
or a salt thereof, wherein:
R₁-R₂₇ are independently selected from the group consisting of hydrogen and deuterium; and
at least one of R₁-R₂₇ is deuterium.

2. The compound as recited in claim 1 wherein:
if R₁₂-R₁₃ and R₂₃-R₂₆ are deuterium, then at least one of R₁-R₁₁, R₁₄-R₂₂ and R₂₇ is deuterium;
if R₂₃-R₂₆ are deuterium, then at least one of R₁-R₂₂ and R₂₇ is deuterium;
if R₂₃-R₂₄ are deuterium, then at least one of R₁-R₂₂ and R₂₅-R₂₇ is deuterium; and
if R₁₂-R₁₃ and R₂₃-R₂₄ are deuterium, then at least one of R₁-R₁₁, R₁₄-R₂₂ and R₂₅-R₂₇ is deuterium.

3. The compound as recited in claim 1 wherein at least one of R₁-R₂₇ independently has deuterium enrichment of no less than about 10%.

4. The compound as recited in claim 1 wherein at least one of R₁-R₂₇ independently has deuterium enrichment of no less than about 50%.

5. The compound as recited in claim 1 wherein at least one of R₁-R₂₇ independently has deuterium enrichment of no less than about 90%.

6. The compound as recited in claim 1 wherein at least one of R₁-R₂₇ independently has deuterium enrichment of no less than about 98%.

7. The compound as recited in claim 1 wherein said compound has a structural formula selected from the group consisting of

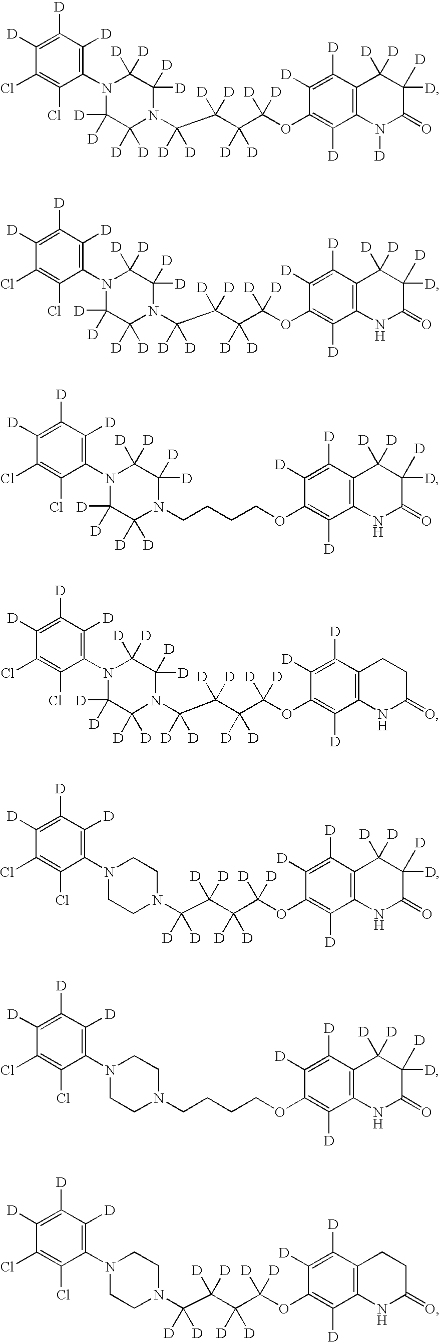

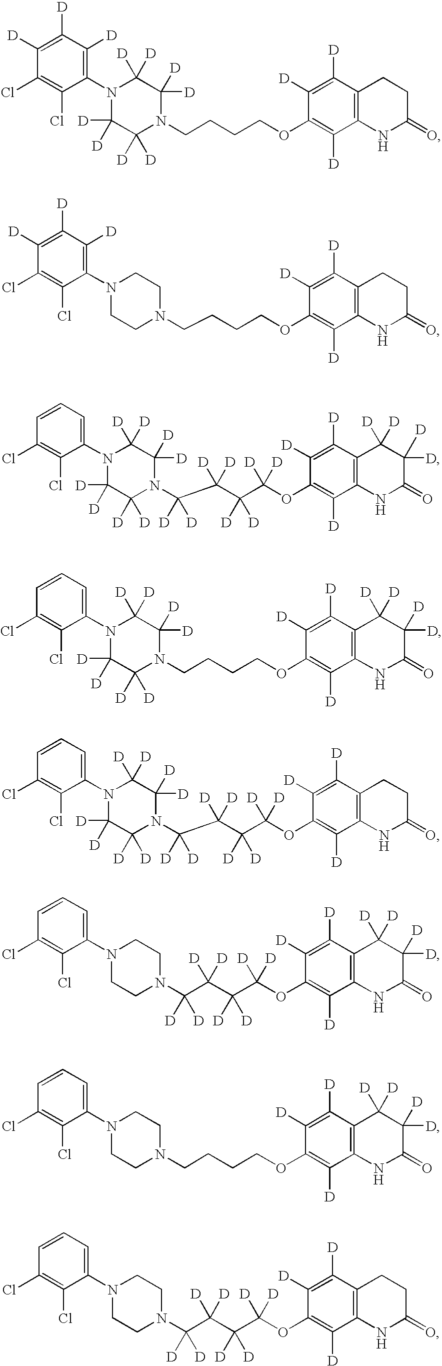

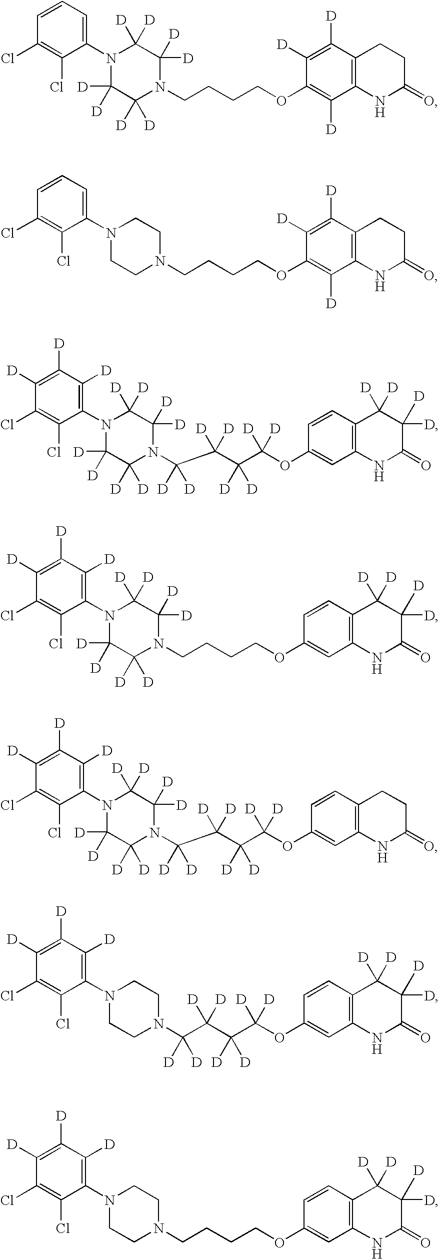

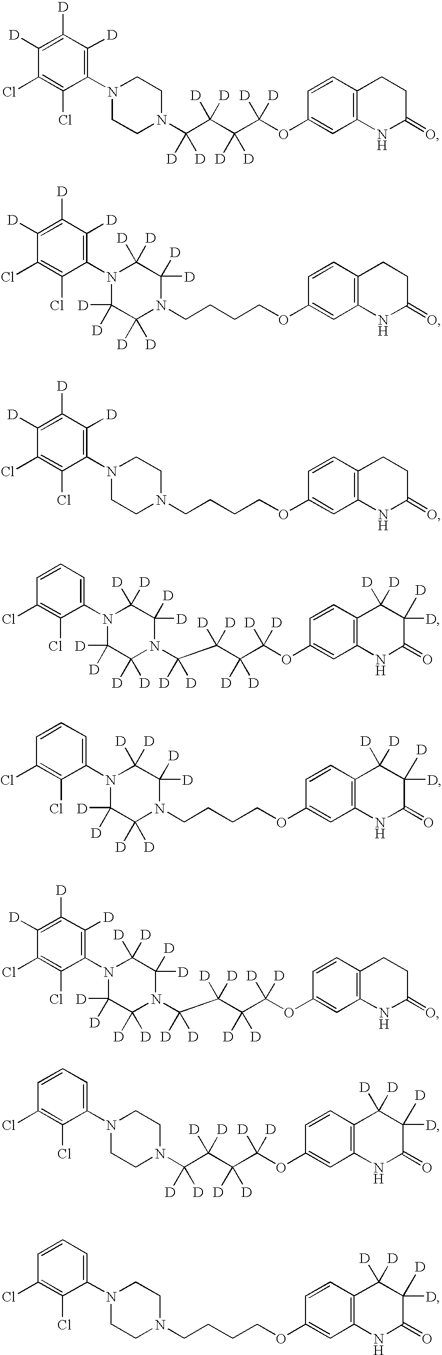

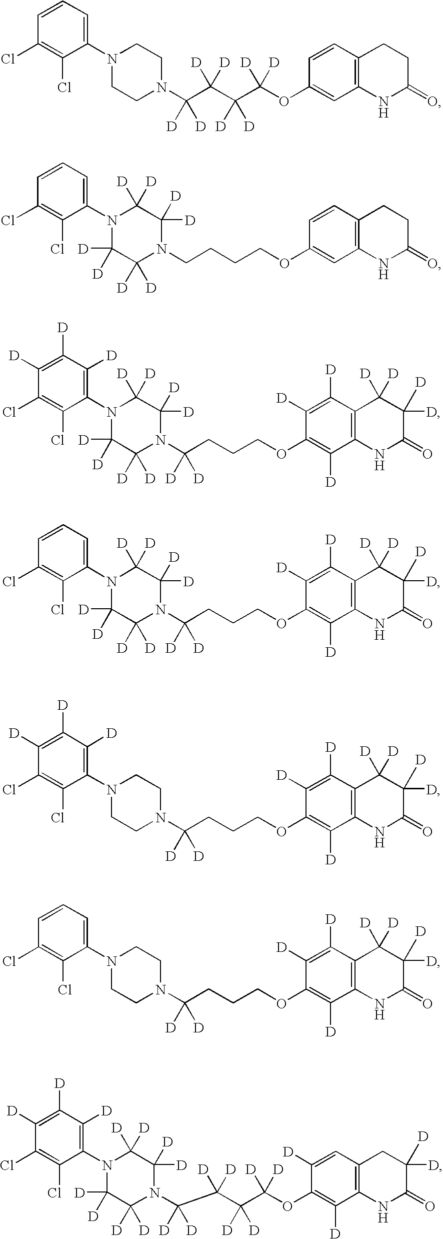

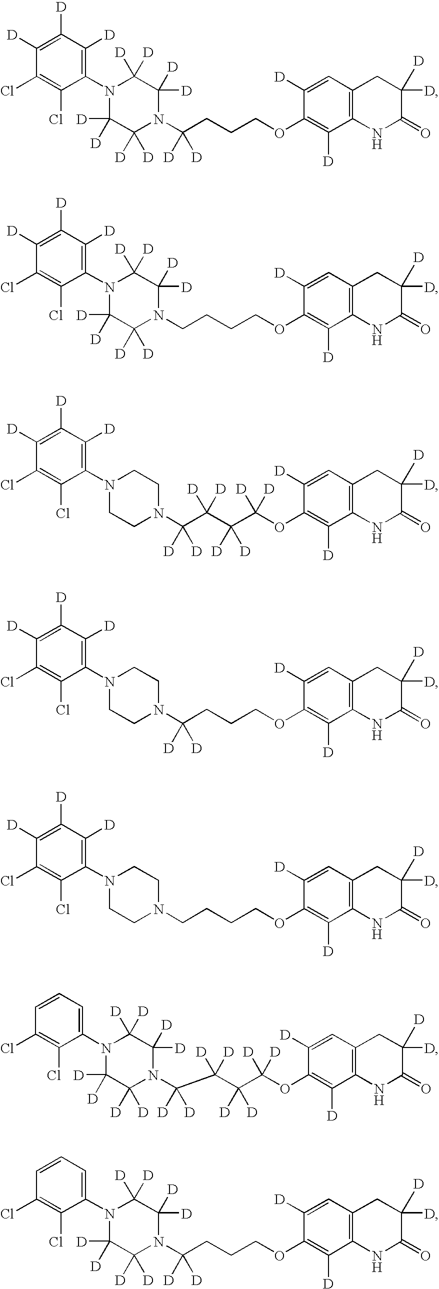

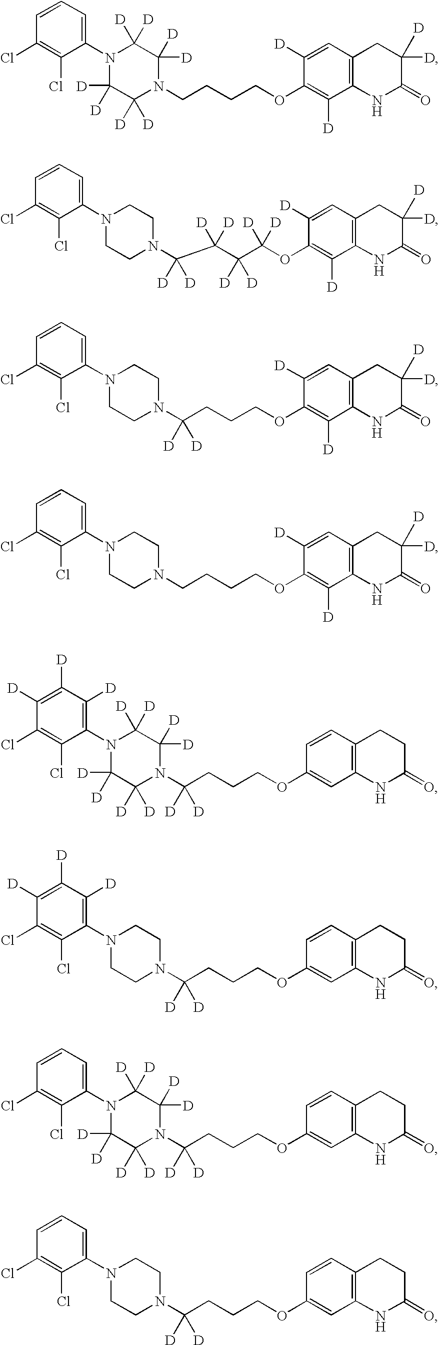

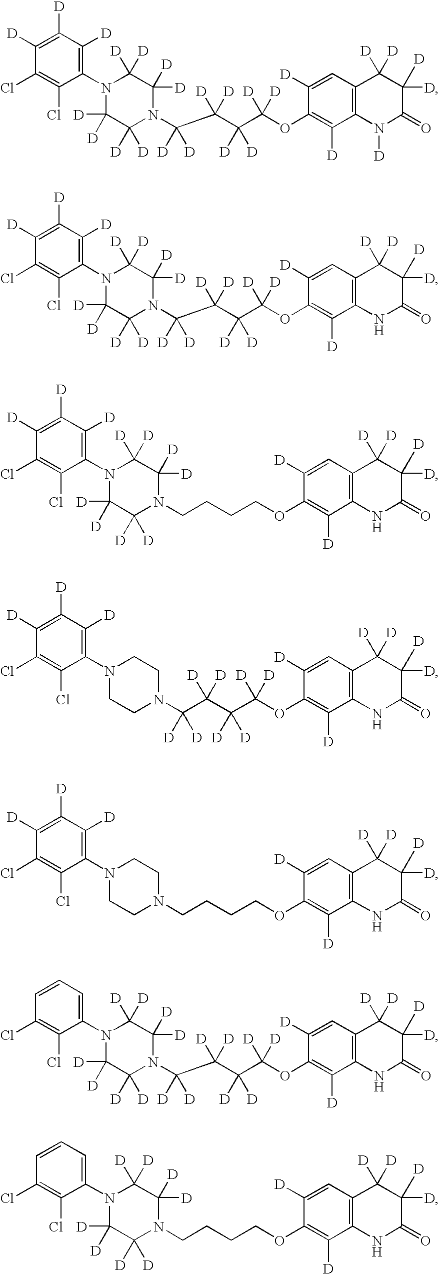

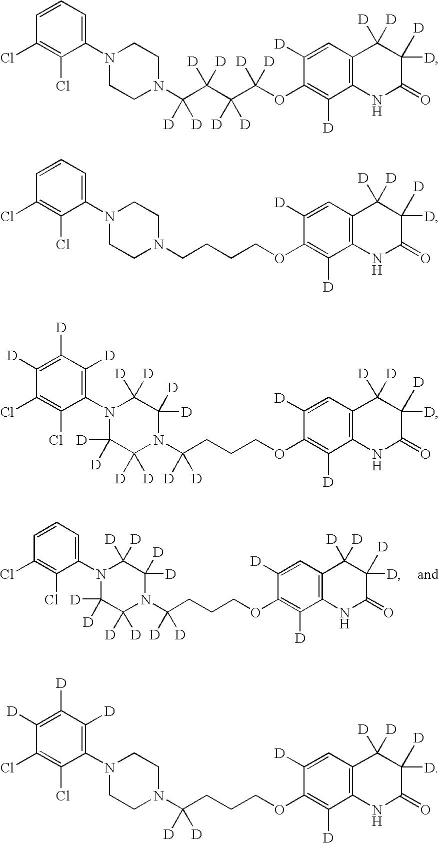

8. The compound as recited in claim 1 wherein said compound has a structural formula selected from the group consisting of

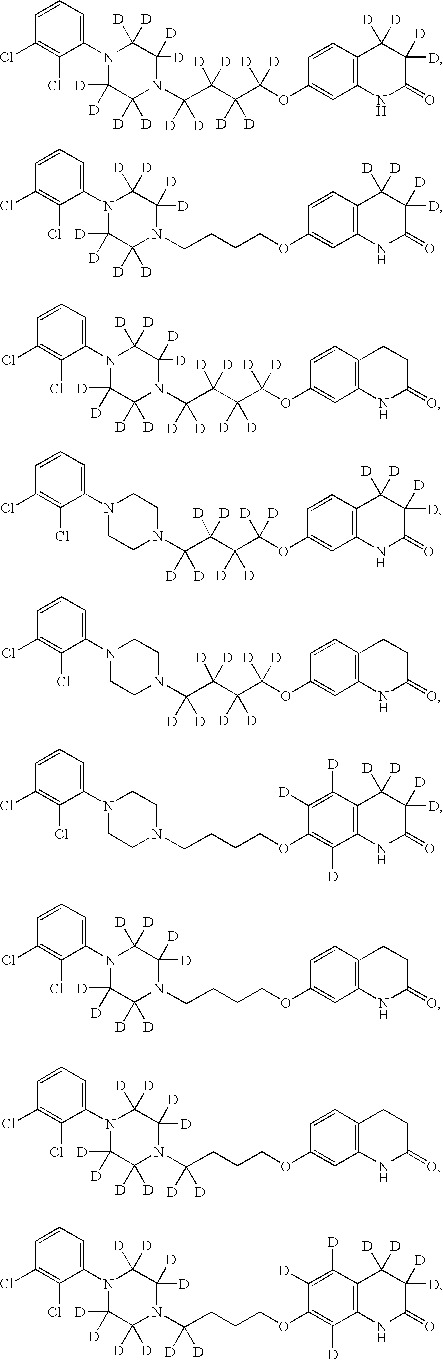

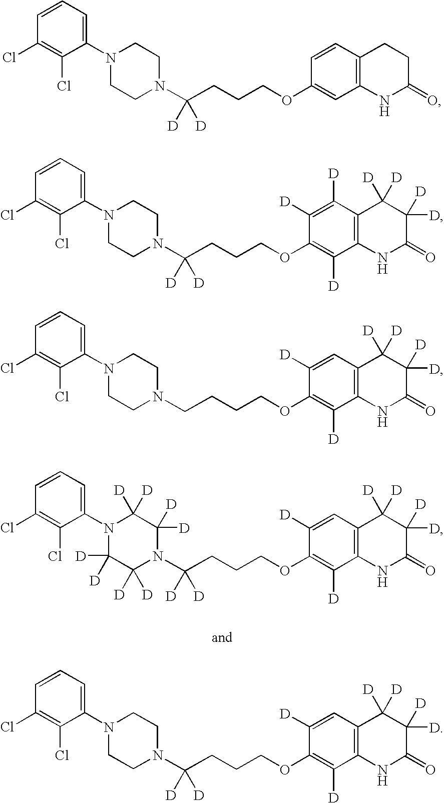

9. The compound as recited in claim 8 wherein each position represented as D has deuterium enrichment of no less than about 10%.

10. The compound as recited in claim 8 wherein each position represented as D has deuterium enrichment of no less than about 50%.

11. The compound as recited in claim 8 wherein each position represented as D has deuterium enrichment of no less than about 90%.

12. The compound as recited in claim 8 wherein each position represented as D has deuterium enrichment of no less than about 98%.

13. The compound as recited in claim 1 wherein said compound has the structural formula:

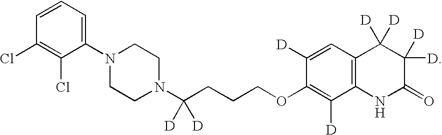

14. A pharmaceutical composition comprising a compound as recited in claim 1 together with a pharmaceutically acceptable carrier.

15. A method of treatment of a a D2 receptor-mediated disorder, a 5-HT1A receptor-mediated disorder, or a 5-HT2A receptor-mediated disorder comprising the administration of a therapeutically effective amount of a compound as recited in claim 1 to a patient in need thereof.

16. The method as recited in claim 15 wherein said disorder is selected from the group consisting of schizophrenia, attention deficit hyperactivity disorder, autism, drug dependence, acute bipolar mania, bipolar disorder, and major depressive disorder.

17. The method as recited in claim 15 further comprising the administration of an additional therapeutic agent.

18. The method as recited in claim 17 wherein said additional therapeutic agent is selected from the group consisting of antidepressants and antipsychotics.

19. The method as recited in claim 17 wherein said additional therapeutic agent is selected from the group consisting of citalopram, escitalopram, paroxetine, fluotexine, fluvoxamine, sertraline, isocarboxazid, moclobemide, phenelzine, tranylcypromine, amitriptyline, clomipramine, desipramine, dosulepin, imipramine, nortriptyline, protriptyline, trimipramine, lofepramine, maprotiline, amoxapine, mianserin, mirtazapine, duloxetine, nefazodone, reboxetine, trazodone, venlafaxine, tianeptine, and milnacipran.

20. The method as recited in claim 17 wherein said additional therapeutic agent is selected from the group consisting of haloperidol, chlorpromazine, fluphenazine, perphenazine, prochlorperazine, thioridazine, trifluoperazine, mesoridazine, promazine, triflupromazine, levomepromazine, promethazine, chlorprothixene, flupenthixol, thiothixene, zuclopenthixol, clozapine, olanzapine, quetiapine, risperidone, ziprasidone, amisulpride, paliperidone, bifeprunox, norclozapine, tetrabenazine, and cannabidiol.

21. The method as recited in claim 17 wherein said additional therapeutic agent is selected from the group consisting of lithium and valproate.

22. The method as recited in claim 15, further resulting in at least one effect selected from the group consisting of:
a. decreased inter-individual variation in plasma levels of said compound or a metabolite thereof as compared to the non-isotopically enriched compound;
b. increased average plasma levels of said compound per dosage unit thereof as compared to the non-isotopically enriched compound;
c. decreased average plasma levels of at least one metabolite of said compound per dosage unit thereof as compared to the non-isotopically enriched compound;
d. increased average plasma levels of at least one metabolite of said compound per dosage unit thereof as compared to the non-isotopically enriched compound; and
e. an improved clinical effect during the treatment in said subject per dosage unit thereof as compared to the non-isotopically enriched compound.

23. The method as recited in claim 15, further resulting in at least two effects selected from the group consisting of:
a. decreased inter-individual variation in plasma levels of said compound or a metabolite thereof as compared to the non-isotopically enriched compound;
b. increased average plasma levels of said compound per dosage unit thereof as compared to the non-isotopically enriched compound;
c. decreased average plasma levels of at least one metabolite of said compound per dosage unit thereof as compared to the non-isotopically enriched compound;
d. increased average plasma levels of at least one metabolite of said compound per dosage unit thereof as compared to the non-isotopically enriched compound; and
e. an improved clinical effect during the treatment in said subject per dosage unit thereof as compared to the non-isotopically enriched compound.

24. The method as recited in claim 15, wherein the method effects a decreased metabolism of the compound per dosage unit thereof by at least one polymorphically-expressed cytochrome P₄₅₀ isoform in the subject, as compared to the corresponding non-isotopically enriched compound.

25. The method as recited in claim 24, wherein the cytochrome P₄₅₀ isoform is selected from the group consisting of CYP2C8, CYP2C9, CYP2C19, and CYP2D6.

26. The method as recited claim 15, wherein said compound is characterized by decreased inhibition of at least one cytochrome P₄₅₀ or monoamine oxidase isoform in said subject per dosage unit thereof as compared to the non-isotopically enriched compound.

27. The method as recited in claim 26, wherein said cytochrome P₄₅₀ or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP12A1, CYP11B1, CYP11B2, CYP17, CYP19, CYP21, CYP24, CYP26A1, CYP26B1, CYP27A1, CYP27B1, CYP39, CYP46, CYP51, MAO_{A}, and MAO_{B}.

28. The method as recited in claim 15, wherein the method reduces a deleterious change in a diagnostic hepatobiliary function endpoint, as compared to the corresponding non-isotopically enriched compound.

29. The method as recited in claim 28, wherein the diagnostic hepatobiliary function endpoint is selected from the group consisting of alanine aminotransferase (“ALT”), serum glutamic-pyruvic transaminase (“SGPT”), aspartate aminotransferase (“AST,” “SGOT”), ALT/AST ratios, serum aldolase, alkaline phosphatase (“ALP”), ammonia levels, bilirubin, gamma-glutamyl transpeptidase (“GGTP,” “γ-GTP,” “GGT”), leucine aminopeptidase (“LAP”), liver biopsy, liver ultrasonography, liver nuclear scan, 5′-nucleotidase, and blood protein.

30. A compound as recited in claim 1 for use as a medicament.

31. A compound as recited in claim 1 for use in the manufacture of a medicament for the prevention or treatment of a disorder ameliorated by the modulation of D2 receptors, 5-HT1A receptors, or 5-HT2A receptors.

32. A compound of formula II: 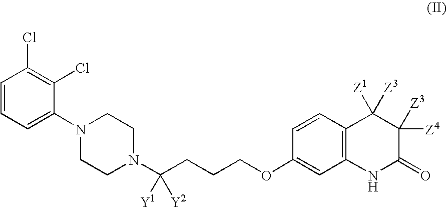
or a pharmaceutically acceptable salt, solvate, or hydrate thereof, wherein:
each Y is independently selected from hydrogen and deuterium; and
each Z is independently selected from hydrogen and deuterium, wherein:
at least one Y or Z is deuterium.

33. The compound of claim 32, wherein each of Y¹ and Y² is the same.

34. The compound of claim 33, wherein Y¹ and Y² are simultaneously deuterium.

35. The compound of claim 32, wherein each of Z¹ and Z² is the same.

36. The compound of claim 35, wherein Z¹ and Z² are simultaneously deuterium.

37. The compound of claim 32, wherein each of Z³ and Z⁴ is the same.

38. The compound of claim 37, wherein Z³ and Z⁴ are simultaneously deuterium.

39. The compound of claim 32, wherein at least two of Y¹, Y², Z¹, Z², Z³, and Z⁴ are deuterium.

40. The compound of claim 39, wherein at least three of Y¹, Y², Z¹, Z², Z³, and Z⁴ are deuterium.

41. A compound of formula III: 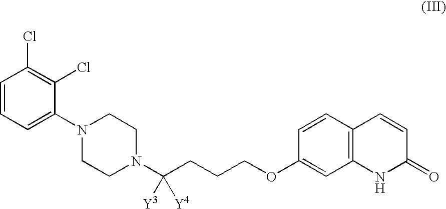
or a pharmaceutically acceptable salt, solvate, or hydrate thereof, wherein:
Y³ is deuterium; and
Y⁴ is selected from hydrogen or deuterium.

42. The compound of claim 41, wherein Y⁴ is deuterium.

43. A compound of this invention selected from the group consisting of: 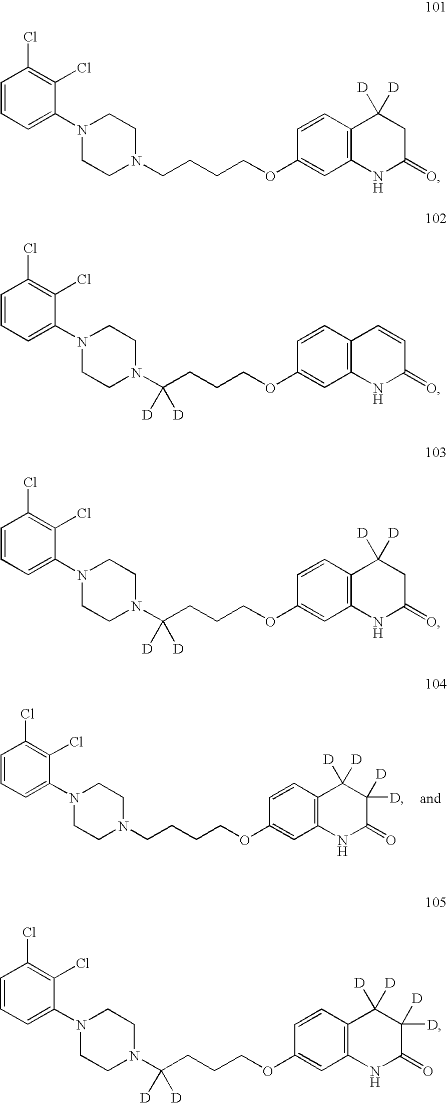
or a HCl salt thereof.

44. The compound of claim 32, wherein each atom not designated as deuterium is present at its naturally abundant isotopic state.

45. A composition comprising:
a. an effective amount of a compound of claim 32; and
b. an acceptable carrier.

46. The composition of claim 45, wherein said composition is formulated for pharmaceutical use; and the carrier is a pharmaceutically acceptable carrier.

47. The composition of claim 46, wherein the composition is formulated for oral administration.

48. The composition of claim 47, wherein the composition is in the form of a pill, capsule or tablet.

49. The composition of claim 48 in dosage unit form, comprising from 0.1 to 250 mg of said compound.

50. The composition of claim 49, comprising from 2 to 50 mg of said compound.

51. The composition of claim 46 further comprising an effective amount of a second therapeutic agent, wherein said second therapeutic agent is useful for treating or preventing a disease or a condition selected from schizophrenia, depression, bipolar depression, depressive disorder, refractive bipolar disorder, autism, alcoholism, cocaine dependency, attention deficit hyperactivity disorder, mood disorders, post traumatic stress disorder, premenstrual dysphoric disorder, nausea, psychotic disorder, tardive dyskinesia, epilepsy, compulsivity, impulsivity, cognition enhancement, weight management, sexual disorders including Hypoactive Sexual Desire Disorder, loss of sexual desire, lack of sexual desire, decreased sexual desire, inhibited sexual desire, loss of libido, libido disturbance, and frigidity.

52. The composition according to claim 51, wherein said second therapeutic agent is selected from a NK3 receptor antagonist; a Gly Transporter Type I inhibitor; memantine; an AMPA receptor potentiator; a GABA modulator, anticonvulsant, or benzodiazepine; an antidepressant; a nicotinic receptor agonist or antagonist; a serotonin reuptake inhibitor; sabcomeline, a M1/M4 receptor agonist; an opioid antagonist; D-cycloserine; lamotrigine; methylphenidate; divalproex; clozapine; H1-receptor; an adenosine A2a receptor antagonist; COX-2 inhibitor; an azabicyclo compound for treating CNS disorders; flibanserin; lithium; a pharmaceutically acceptable salt of any of the foregoing additional therapeutic agents; and combinations of the foregoing therapeutic agents.

53. A substantially isolated isomer of a compound of claim 32.

54. An article of manufacture comprising separate dosage forms of:
a. a compound of claim 32; and
b. a second therapeutic agent, wherein both dosage forms are in a single container.

55. A method for treating a patient suffering from or susceptible to a disorder beneficially treated by an atypical antipsychotic agent, comprising the step of administering to the patient in need thereof a composition according to claim 46.

56. The method according to claim 55, wherein the disorder is selected from schizophrenia, mania or bipolar disorder.

57. The method according to claim 55, wherein the disorder is selected from major depressive disorder, ADHD, autism, conduct disorder, anxiety disorder, social anxiety disorder, substance abuse, prodromal psychosis, Tourette's disorder, Asperger's disorder, pervasive developmental disorder, or alcoholism.

58. The method according to claim 55, comprising the additional step of coadministering to the patient in need thereof a second therapeutic agent selected from a NK3 receptor antagonist; a Gly Transporter Type I inhibitor; memantine; an AMPA receptor potentiator; a GABA modulator, anticonvulsant, or benzodiazepine; an antidepressant; a nicotinic receptor agonist or antagonist; a serotonin reuptake inhibitor; sabcomeline, a M1/M4 receptor agonist; an opioid antagonist; D-cycloserine; lamotrigine; methylphenidate; divalproex; clozapine; H1-receptor; an adenosine A2a receptor antagonist; COX-2 inhibitor; an azabicyclo compound for treating CNS disorders; flibanserin; lithium; a pharmaceutically acceptable salt of any of the foregoing second therapeutic agents; or combinations of the foregoing second therapeutic agents.

59. The method of claim 58, wherein:
a. the patient suffering from or susceptible to schizophrenia or bipolar disorder; and the second therapeutic agent is selected from clozapine, depakote ER, or lamotrigine;
b. the patient suffering from or susceptible to ADHD; and the second therapeutic agent is methylphenidate;
c. the patient suffering from or susceptible to autism; and the second therapeutic agent is D-cycloserine; or
d. the patient suffering from or susceptible to alcoholism; and the second therapeutic agent is an opioid antagonist.

60. A pharmaceutical composition for use in the treatment of a condition selected from schizophrenia, bipolar disorder, bipolar mania, autism, alcoholism, agitation, attention deficit/hyperactivity disorder, anxiety, behavioral disorder, dementia, Alzheimer's dementia, Asperger's disorder, conduct disorder, depression, drug dependency, insulin resistance, mania, obsessive-compulsive disorder, Parkinson's disease, psychosis associated with dementia, drug-induced psychosis, pervasive developmental disorder, prodromal schizophrenia, prodromal psychoses, schizoaffective disorder, social anxiety, tic, and Tourette's disorder, said composition comprising a compound of claim 32; and a pharmaceutically acceptable carrier.

61. The composition according to claim 60, wherein the use is the treatment of schizophrenia, mania or bipolar disorder.

62. The composition according to claim 60, additionally comprising a second therapeutic agent selected from clozapine, depakote ER, and lamotrigine.

63. A composition comprising:
a. an effective amount of a compound of claim 41; and
b. an acceptable carrier.

64. The composition of claim 63, wherein said composition is formulated for pharmaceutical use; and the carrier is a pharmaceutically acceptable carrier.

65. The composition of claim 64, wherein the composition is formulated for oral administration.

66. The composition of claim 65, wherein the composition is in the form of a pill, capsule or tablet.

67. The composition of claim 66 in dosage unit form, comprising from 0.1 to 250 mg of said compound.

68. The composition of claim 67, comprising from 2 to 50 mg of said compound.

69. The composition of claim 64 further comprising an effective amount of a second therapeutic agent, wherein said second therapeutic agent is useful for treating or preventing a disease or a condition selected from schizophrenia, depression, bipolar depression, depressive disorder, refractive bipolar disorder, autism, alcoholism, cocaine dependency, attention deficit hyperactivity disorder, mood disorders, post traumatic stress disorder, premenstrual dysphoric disorder, nausea, psychotic disorder, tardive dyskinesia, epilepsy, compulsivity, impulsivity, cognition enhancement, weight management, sexual disorders including Hypoactive Sexual Desire Disorder, loss of sexual desire, lack of sexual desire, decreased sexual desire, inhibited sexual desire, loss of libido, libido disturbance, and frigidity.

70. The composition according to claim 69, wherein said second therapeutic agent is selected from a NK3 receptor antagonist; a Gly Transporter Type I inhibitor; memantine; an AMPA receptor potentiator; a GABA modulator, anticonvulsant, or benzodiazepine; an antidepressant; a nicotinic receptor agonist or antagonist; a serotonin reuptake inhibitor; sabcomeline, a M1/M4 receptor agonist; an opioid antagonist; D-cycloserine; lamotrigine; methylphenidate; divalproex; clozapine; H1-receptor; an adenosine A2a receptor antagonist; COX-2 inhibitor; an azabicyclo compound for treating CNS disorders; flibanserin; lithium; a pharmaceutically acceptable salt of any of the foregoing additional therapeutic agents; and combinations of the foregoing therapeutic agents.

71. A substantially isolated isomer of a compound of claim 41.

72. An article of manufacture comprising separate dosage forms of:
a. a compound of claim 41; and
b. a second therapeutic agent, wherein both dosage forms are in a single container.

73. A method for treating a patient suffering from or susceptible to a disorder beneficially treated by an atypical antipsychotic agent, comprising the step of administering to the patient in need thereof a composition according to claim 64.

74. The method according to claim 73, wherein the disorder is selected from schizophrenia, mania or bipolar disorder.

75. The method according to claim 73, wherein the disorder is selected from major depressive disorder, ADHD, autism, conduct disorder, anxiety disorder, social anxiety disorder, substance abuse, prodromal psychosis, Tourette's disorder, Asperger's disorder, pervasive developmental disorder, or alcoholism.

76. The method according to claim 73, comprising the additional step of coadministering to the patient in need thereof a second therapeutic agent selected from a NK3 receptor antagonist; a Gly Transporter Type I inhibitor; memantine; an AMPA receptor potentiator; a GABA modulator, anticonvulsant, or benzodiazepine; an antidepressant; a nicotinic receptor agonist or antagonist; a serotonin reuptake inhibitor; sabcomeline, a M1/M4 receptor agonist; an opioid antagonist; D-cycloserine; lamotrigine; methylphenidate; divalproex; clozapine; H1-receptor; an adenosine A2a receptor antagonist; COX-2 inhibitor; an azabicyclo compound for treating CNS disorders; flibanserin; lithium; a pharmaceutically acceptable salt of any of the foregoing second therapeutic agents; or combinations of the foregoing second therapeutic agents.

77. The method of claim 76, wherein:
a. the patient suffering from or susceptible to schizophrenia or bipolar disorder; and the second therapeutic agent is selected from clozapine, depakote ER, or lamotrigine;
b. the patient suffering from or susceptible to ADHD; and the second therapeutic agent is methylphenidate;
c. the patient suffering from or susceptible to autism; and the second therapeutic agent is D-cycloserine; or
d. the patient suffering from or susceptible to alcoholism; and the second therapeutic agent is an opioid antagonist.

78. A pharmaceutical composition for use in the treatment of a condition selected from schizophrenia, bipolar disorder, bipolar mania, autism, alcoholism, agitation, attention deficit/hyperactivity disorder, anxiety, behavioral disorder, dementia, Alzheimer's dementia, Asperger's disorder, conduct disorder, depression, drug dependency, insulin resistance, mania, obsessive-compulsive disorder, Parkinson's disease, psychosis associated with dementia, drug-induced psychosis, pervasive developmental disorder, prodromal schizophrenia, prodromal psychoses, schizoaffective disorder, social anxiety, tic, and Tourette's disorder, said composition comprising a compound of claim 41; and a pharmaceutically acceptable carrier.

79. The composition according to claim 78, wherein the use is the treatment of schizophrenia, mania or bipolar disorder.

80. The composition according to claim 78, additionally comprising a second therapeutic agent selected from clozapine, depakote ER, and lamotrigine.

81. A deuterium-enriched compound of formula IV or a pharmaceutically acceptable salt thereof: 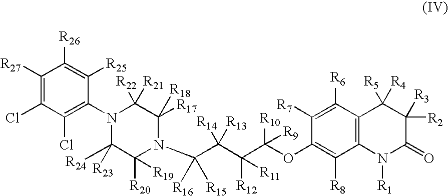
wherein R₁-R₂₇ are independently selected from H and D;
and the abundance of deuterium in R₁-R₂₇ is at least 4%.

82. A deuterium-enriched compound of claim 81, wherein the abundance of deuterium in R₁-R₂₇ is selected from at least 4%, at least 7%, at least 15%, at least 22%, at least 30%, at least 37%, at least 44%, at least 52%, at least 59%, at least 67%, at least 74%, at least 81%, at least 89%, at least 96%, and 100%.

83. A deuterium-enriched compound of claim 81, wherein the abundance of deuterium in R₁ is 100%.

84. A deuterium-enriched compound of claim 81, wherein the abundance of deuterium in R₂-R₃ is selected from at least 50% and 100%.

85. A deuterium-enriched compound of claim 81, wherein the abundance of deuterium in R₆-R₈ and R₂₅-R₂₇ is selected from at least 17%, at least 33%, at least 50%, at least 67%, at least 83%, and 100%.

86. A deuterium-enriched compound of claim 81, wherein the abundance of deuterium in R₁ and R₂-R₃ is selected from at least 33%, at least 67%, and 100%.

87. A deuterium-enriched compound of claim 81, wherein the abundance of deuterium in R₁, R₆-R₈, and R₂₅-R₂₇ is selected from at least 14%, at least 29%, at least 43%, at least 57%, at least 71%, at least 86%, and 100%.

88. A deuterium-enriched compound of claim 81, wherein the abundance of deuterium in R₂-R₃, R₆-R₈, and R₂₅-R₂₇ is selected from at least 13%, at least 25%, at least 38%, at least 50%, at least 63%, at least 75%, at least 88%, and 100%.

89. A deuterium-enriched compound of claim 81, wherein the abundance of deuterium in R₁, R₂-R₃, R₆-R₈, and R₂₅-R₂₇ is selected from at least 11%, at least 22%, at least 33%, at least 44%, at least 56%, at least 67%, at least 78%, and 100%.

90. A deuterium-enriched compound of claim 81, wherein the abundance of deuterium in R₆-R₂₇ is selected from at least 5%, at least 9%, at least 14%, at least 18%, at least 23%, at least 27%, at least 32%, at least 36%, at least 41%, at least 45%, at least 50%, at least 55%, at least 59%, at least 64%, at least 68%, at least 73%, at least 77%, at least 82%, at least 86%, at least 91%, at least 95%, and 100%.

91. A deuterium-enriched compound of claim 81, wherein the abundance of deuterium in R₂-R₅ is selected from at least 25%, at least 50%, at least 75%, and 100%.

92. A deuterium-enriched compound of claim 81, wherein the abundance of deuterium in R₉-R₁₆ is selected from at least 13%, at least 25%, at least 38%, at least 50%, at least 63%, at least 75%, at least 88%, and 100%.

93. A deuterium-enriched compound of claim 81, wherein the abundance of deuterium in R₁₇-R₂₄ is selected from at least 13%, at least 25%, at least 38%, at least 50%, at least 63%, at least 75%, at least 88%, and 100%.

94. A deuterium-enriched compound of claim 81, wherein said compound is selected from the group consisting of:

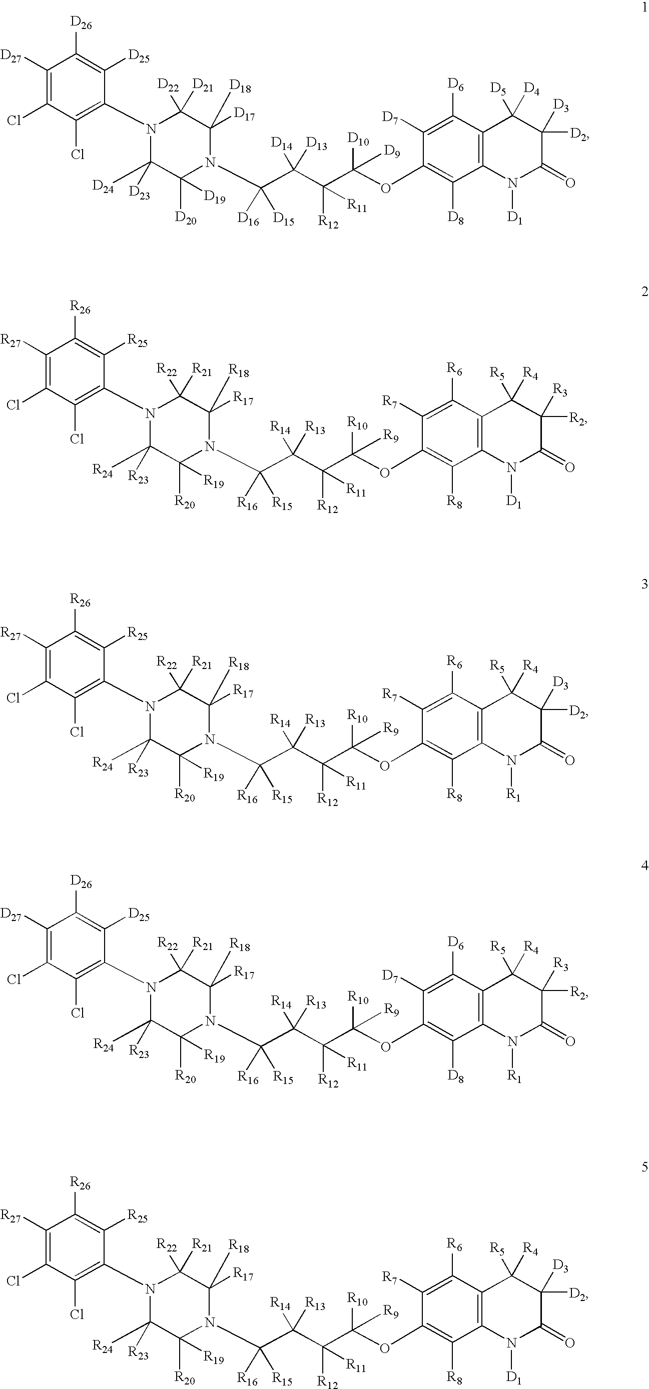

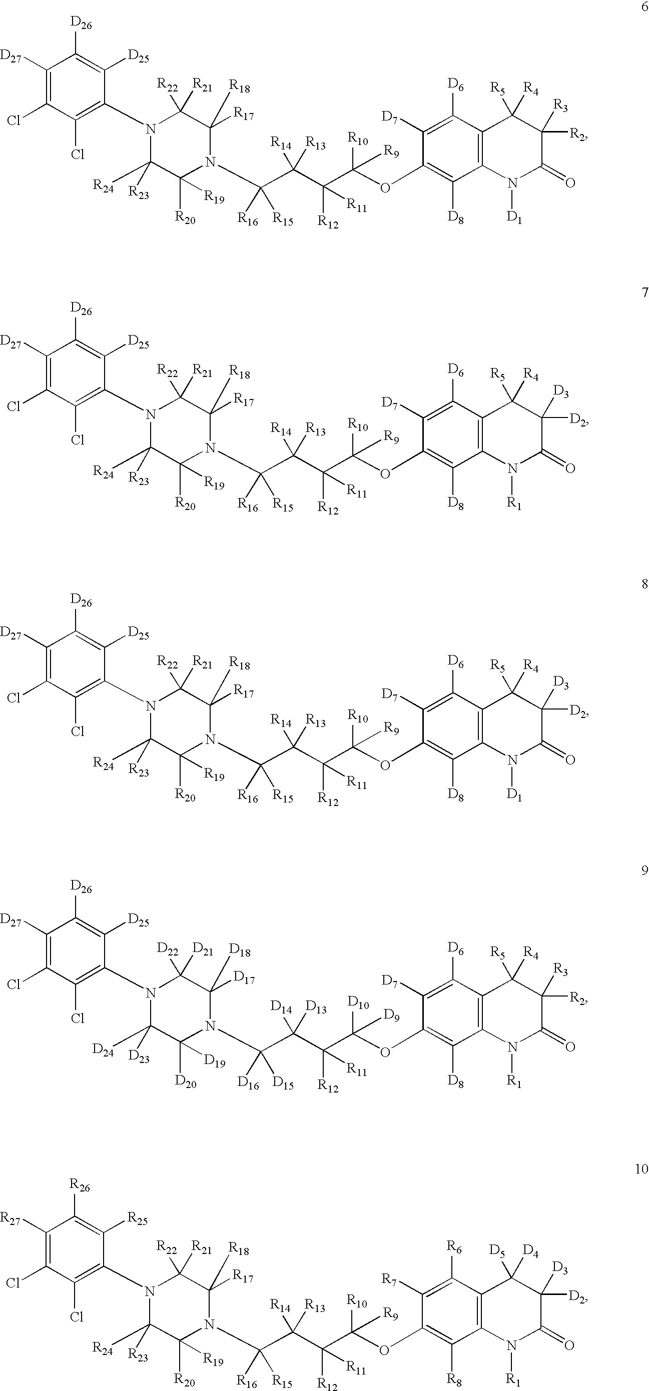

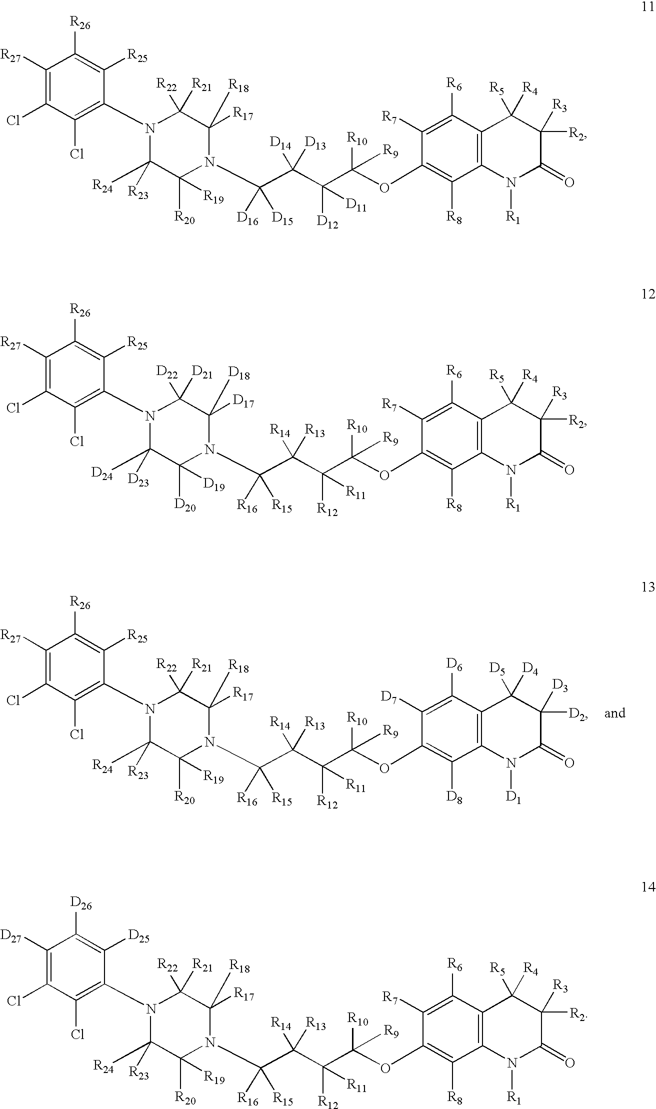

95. A deuterium-enriched compound of claim 81, wherein said compound is selected from the group consisting of:

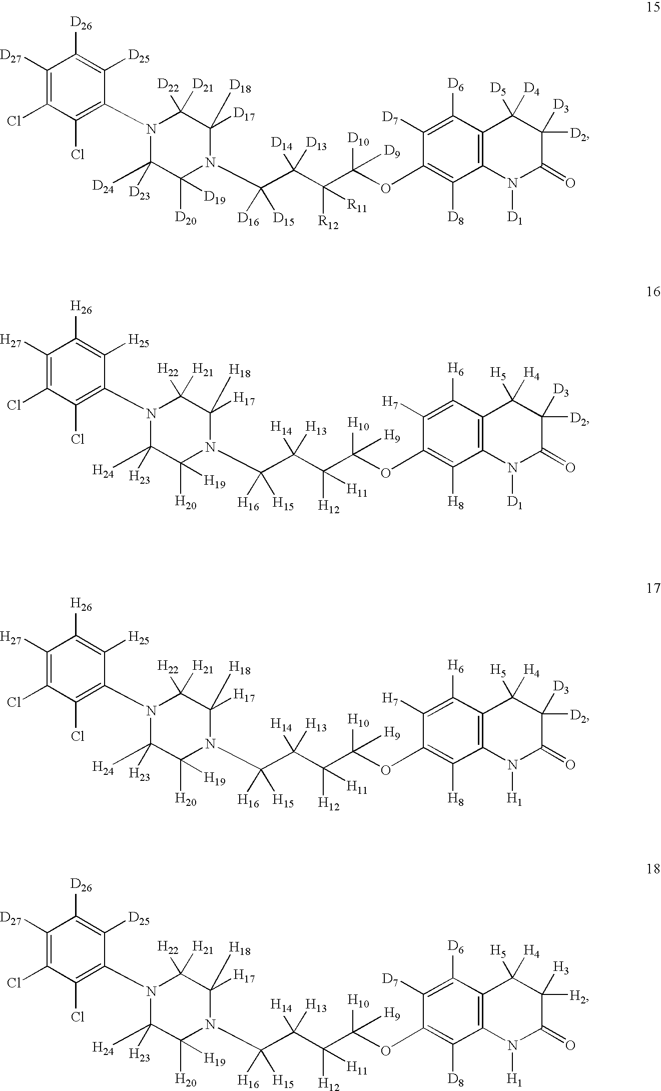

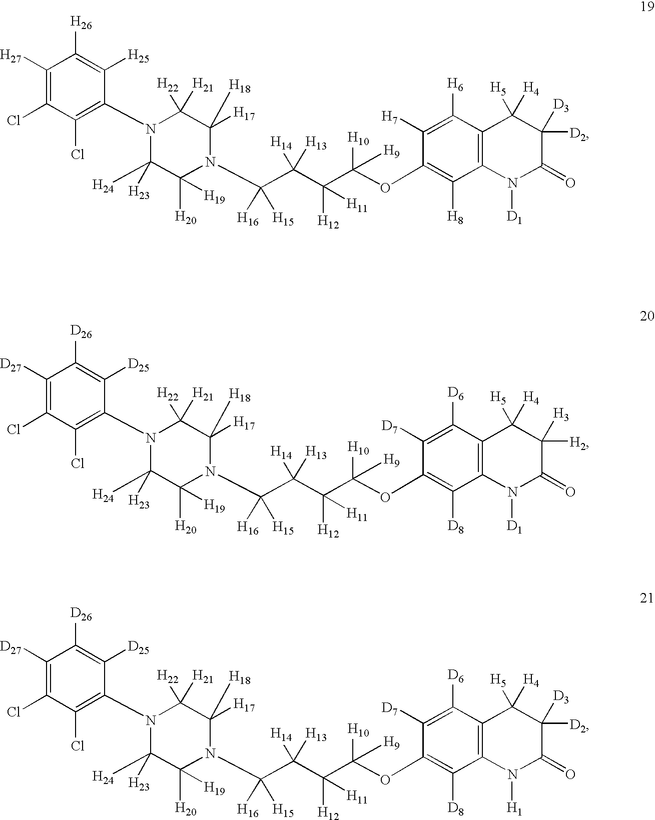

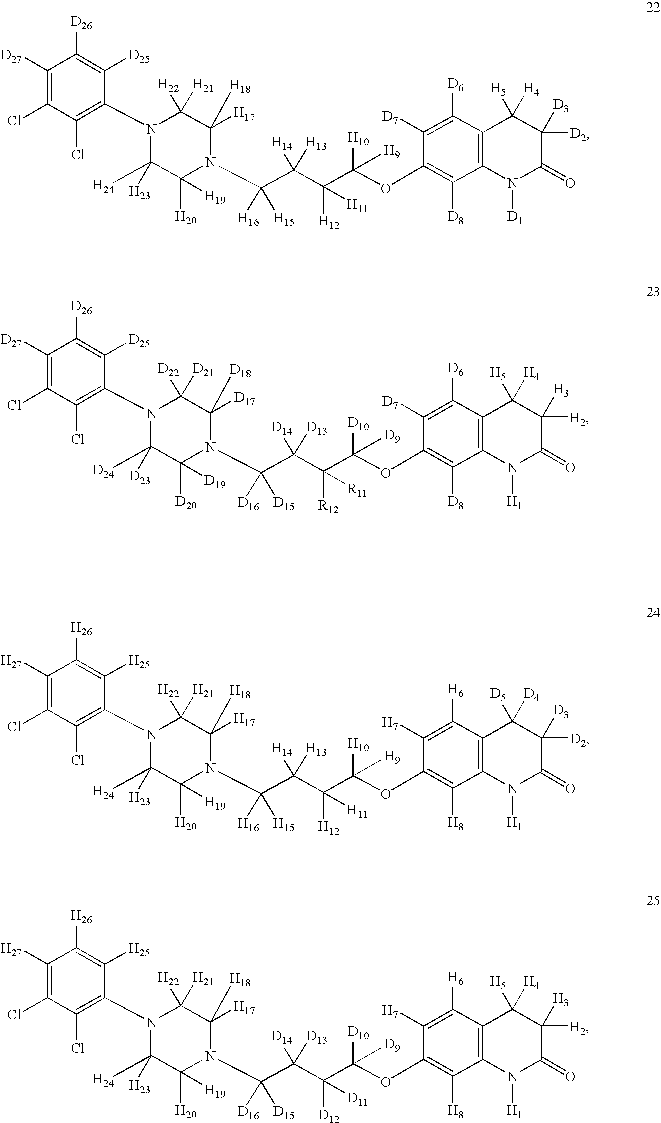

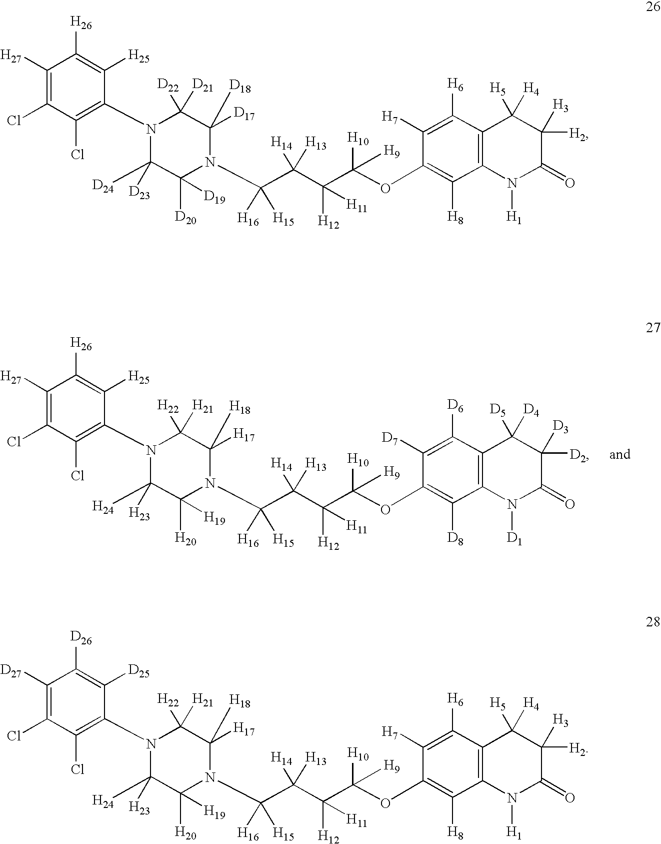

96. An isolated deuterium-enriched compound of structural formula IV, or a pharmaceutically acceptable salt thereof: 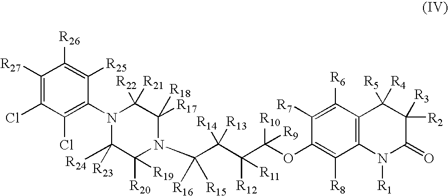
wherein R₁-R₂₇ are independently selected from H and D;
and the abundance of deuterium in R₁-R₂₇ is at least 4%.

97. An isolated deuterium-enriched compound of claim 96, wherein the abundance of deuterium in R₁-R₂₇ is selected from at least 4%, at least 7%, at least 15%, at least 22%, at least 30%, at least 37%, at least 44%, at least 52%, at least 59%, at least 67%, at least 74%, at least 81%, at least 89%, at least 96%, and 100%.

98. An isolated deuterium-enriched compound of claim 96, wherein the abundance of deuterium in R₁ is 100%.

99. An isolated deuterium-enriched compound of claim 96, wherein the abundance of deuterium in R₂-R₃ is selected from at least 50% and 100%.

100. An isolated deuterium-enriched compound of claim 96, wherein the abundance of deuterium in R₆-R₈ and R₂₅-R₂₇ is selected from at least 17%, at least 33%, at least 50%, at least 67%, at least 83%, and 100%.

101. An isolated deuterium-enriched compound of claim 96, wherein the abundance of deuterium in R₁ and R₂-R₃ is selected from at least 33%, at least 67%, and 100%.

102. An isolated deuterium-enriched compound of claim 96, wherein the abundance of deuterium in R₁, R₆-R₈, and R₂₅-R₂₇ is selected from at least 14%, at least 29%, at least 43%, at least 57%, at least 71%, at least 86%, and 100%.

103. An isolated deuterium-enriched compound of claim 96, wherein the abundance of deuterium in R₂-R₃, R₆-R₈, and R₂₅-R₂₇ is selected from at least 13%, at least 25%, at least 38%, at least 50%, at least 63%, at least 75%, at least 88%, and 100%.

104. An isolated deuterium-enriched compound of claim 96, wherein the abundance of deuterium in R₁, R₂-R₃, R₆-R₈, and R₂₅-R₂₇ is selected from at least 11%, at least 22%, at least 33%, at least 44%, at least 56%, at least 67%, at least 78%, and 100%.

105. An isolated deuterium-enriched compound of claim 96, wherein the abundance of deuterium in R₆-R₂₇ is selected from at least 5%, at least 9%, at least 14%, at least 18%, at least 23%, at least 27%, at least 32%, at least 36%, at least 41%, at least 45%, at least 50%, at least 55%, at least 59%, at least 64%, at least 68%, at least 73%, at least 77%, at least 82%, at least 86%, at least 91%, at least 95%, and 100%.

106. An isolated deuterium-enriched compound of claim 96, wherein the abundance of deuterium in R₂-R₅ is selected from at least 25%, at least 50%, at least 75%, and 100%.

107. An isolated deuterium-enriched compound of claim 96, wherein the abundance of deuterium in R₉-R₁₆ is selected from at least 13%, at least 25%, at least 38%, at least 50%, at least 63%, at least 75%, at least 88%, and 100%.

108. An isolated deuterium-enriched compound of claim 96, wherein the abundance of deuterium in R₁₇-R₂₄ is selected from at least 13%, at least 25%, at least 38%, at least 50%, at least 63%, at least 75%, at least 88%, and 100%.

109. An isolated deuterium-enriched compound of claim 96, wherein the compound is selected from the group consisting of:

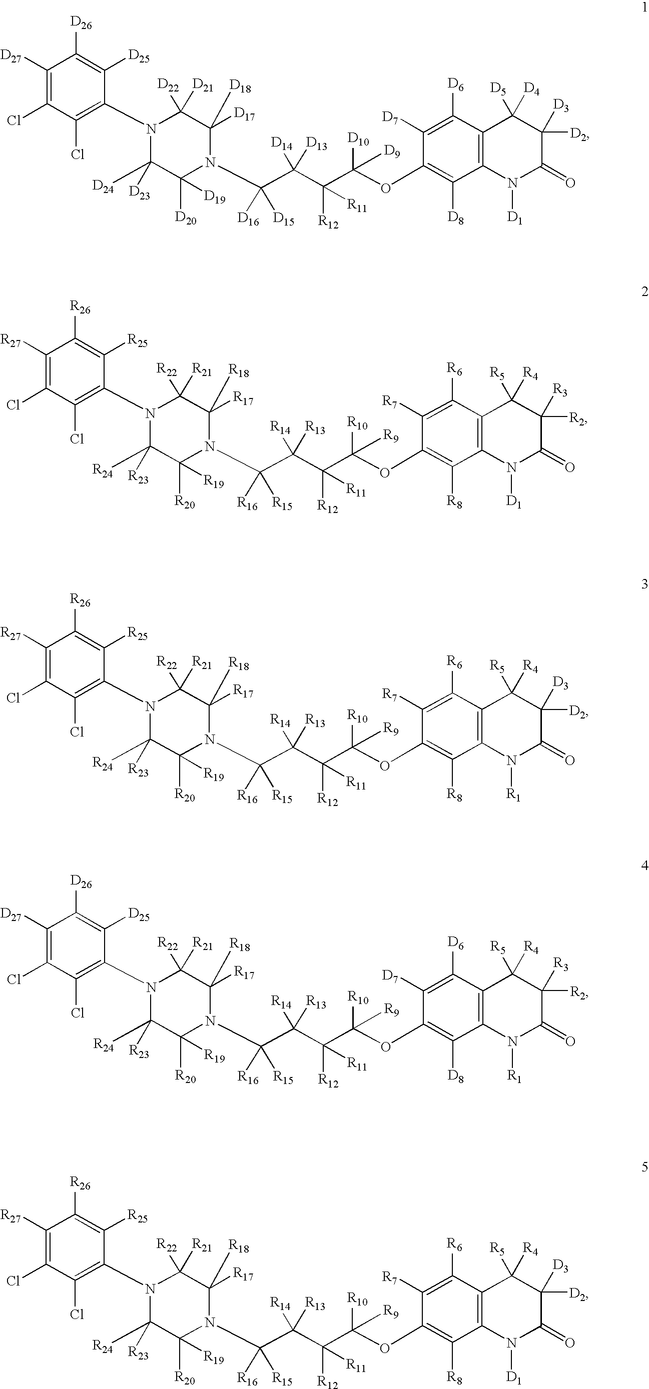

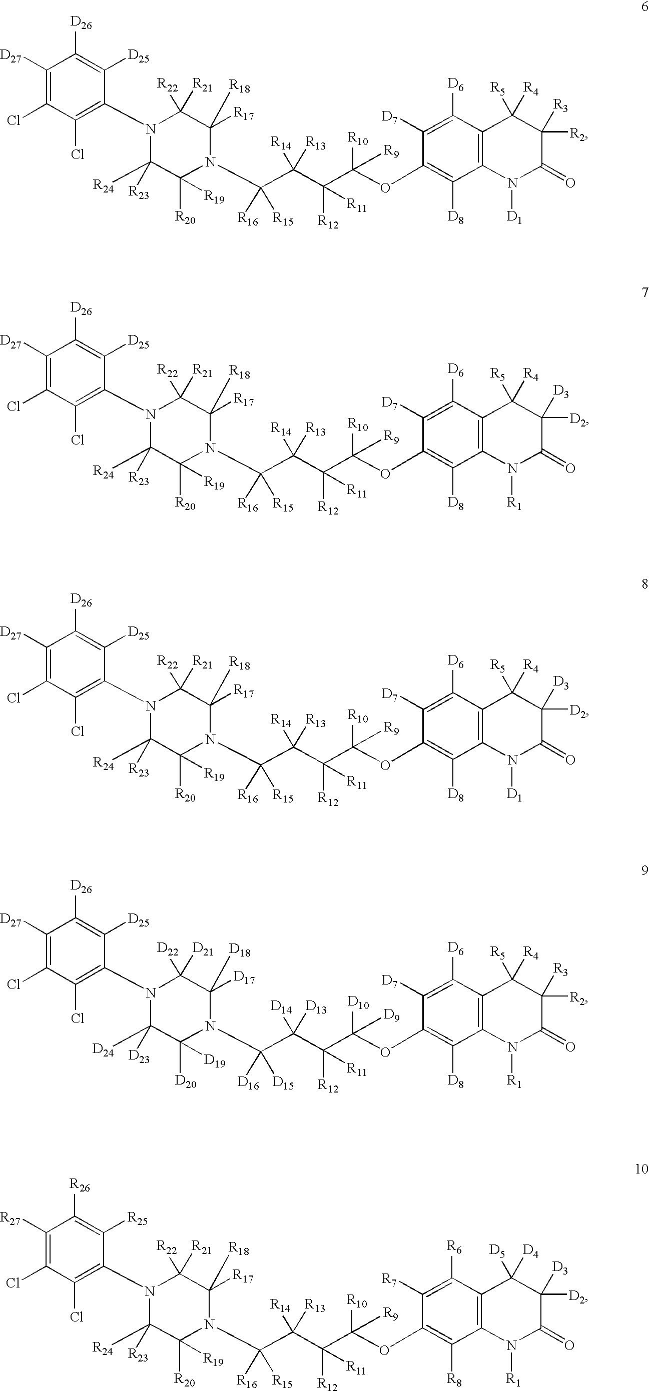

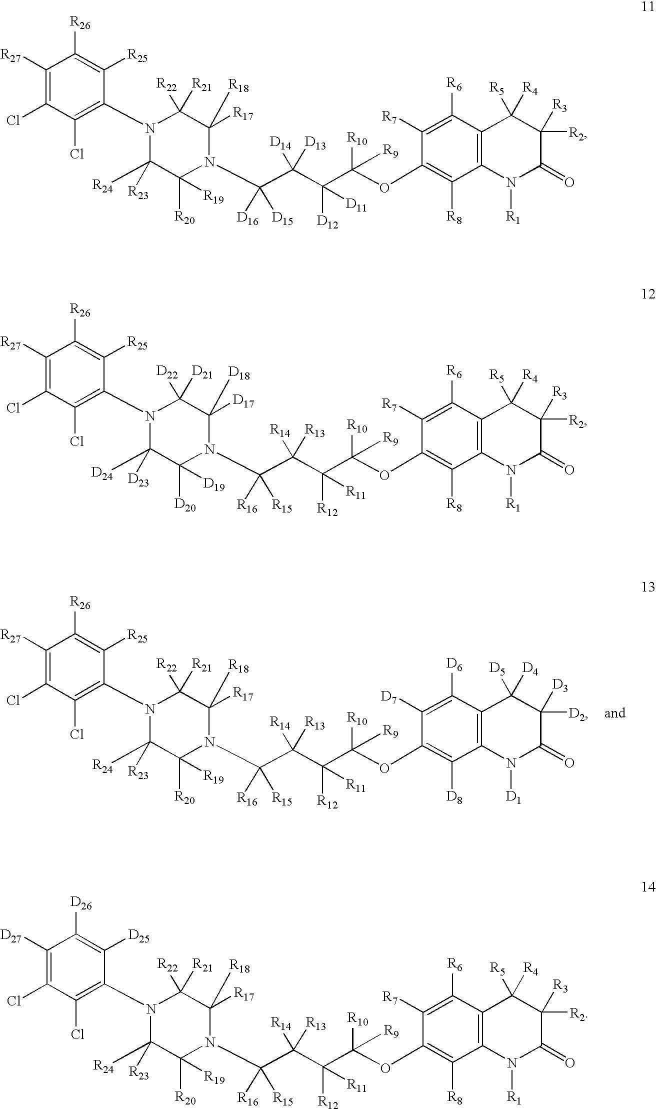

110. An isolated deuterium-enriched compound of claim 96, wherein the compound is selected from the group consisting of:

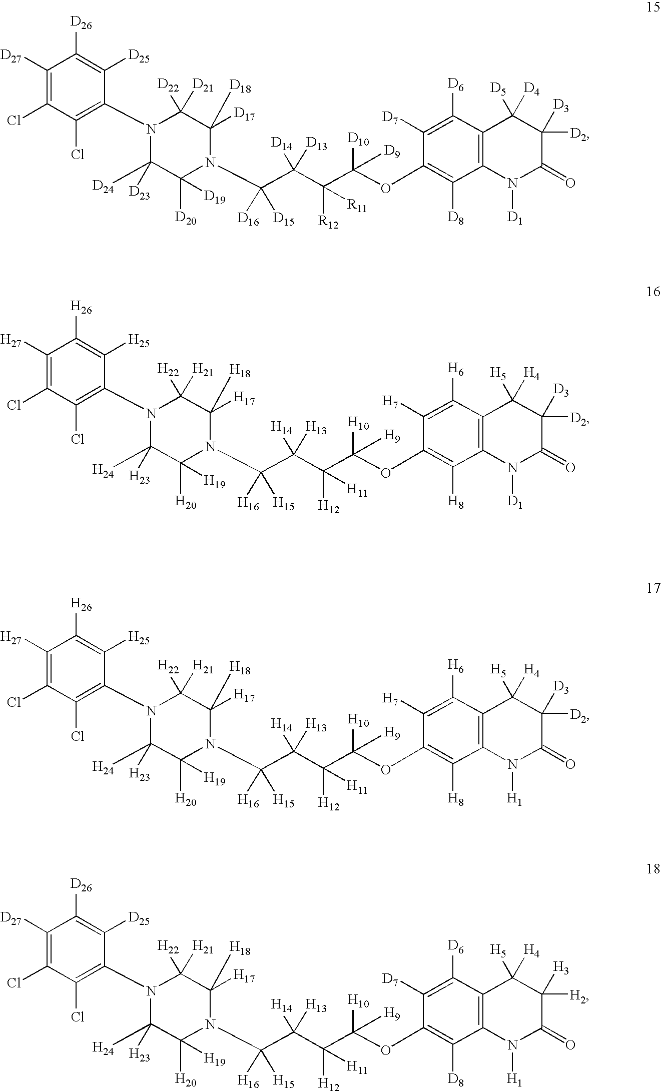

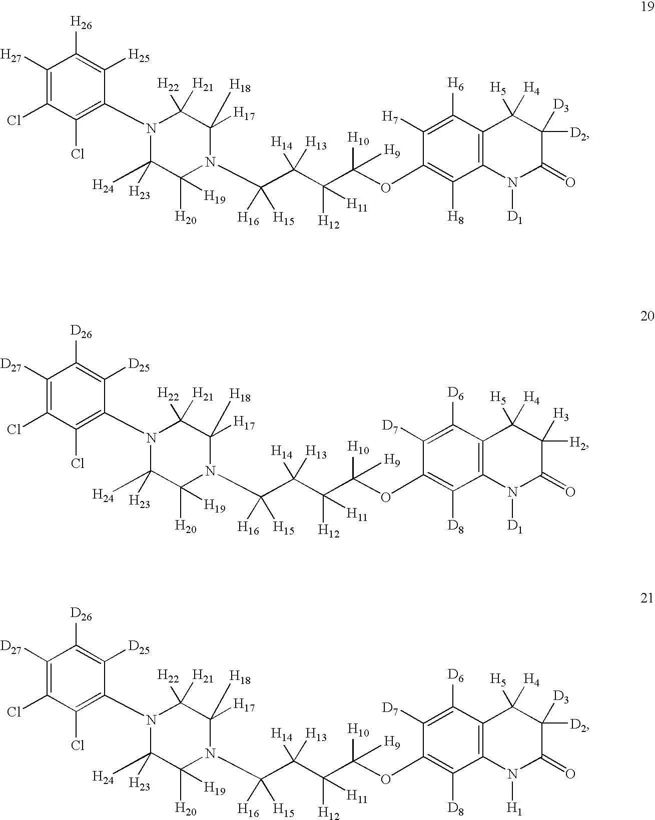

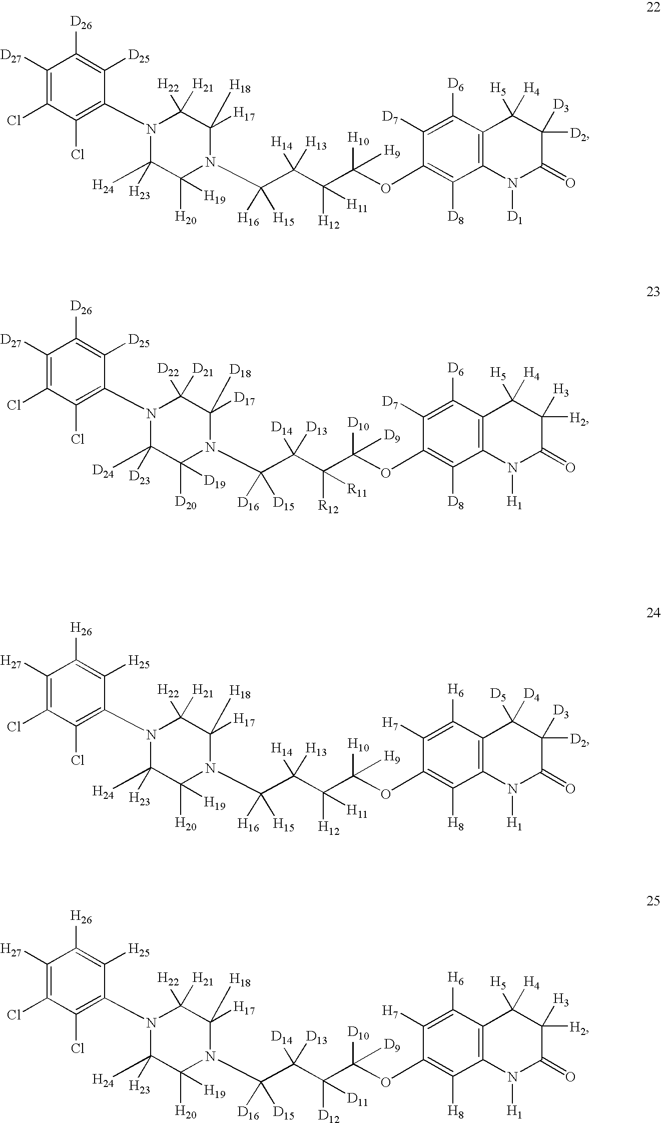

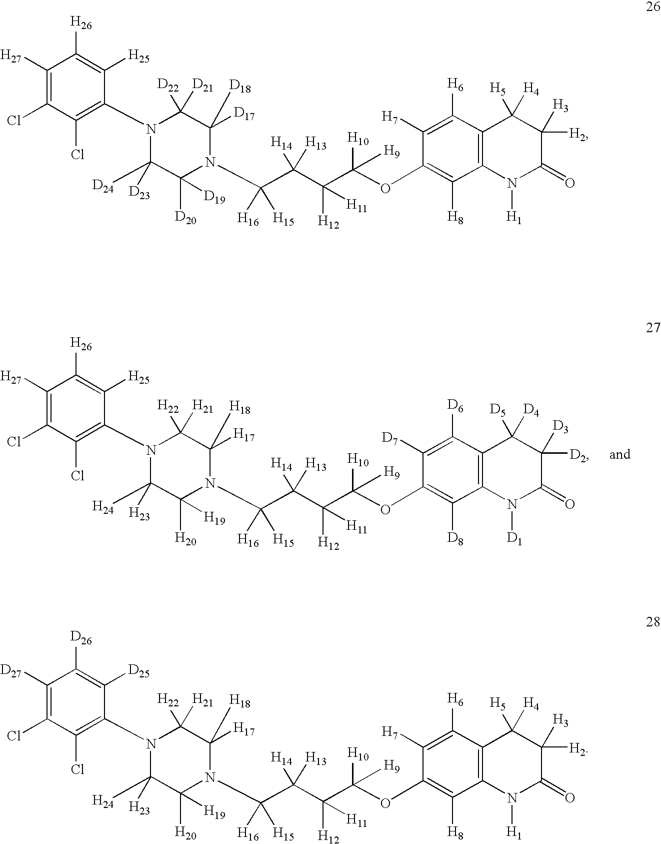

111. A mixture of deuterium-enriched compounds of formula IV, or a pharmaceutically acceptable salt thereof: 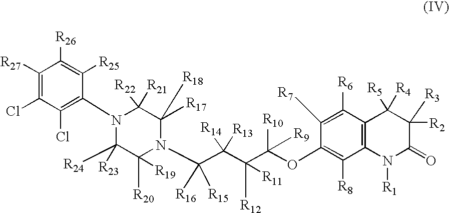
wherein R₁-R₂₇ are independently selected from H and D;
and the abundance of deuterium in R₁-R₂₇ is at least 4%.

112. A mixture of deuterium-enriched compound of claim 111, wherein the abundance of deuterium in R₁-R₂₇ is selected from at least 4%, at least 7%, at least 15%, at least 22%, at least 30%, at least 37%, at least 44%, at least 52%, at least 59%, at least 67%, at least 74%, at least 81%, at least 89%, at least 96%, and 100%.

113. A mixture of deuterium-enriched compound of claim 111, wherein the abundance of deuterium in R₁ is 100%.

114. A mixture of deuterium-enriched compound of claim 111, wherein the abundance of deuterium in R₂-R₃ is selected from at least 50% and 100%.

115. A mixture of deuterium-enriched compound of claim 111, wherein the abundance of deuterium in R₆-R₈ and R₂₅-R₂₇ is selected from at least 17%, at least 33%, at least 50%, at least 67%, at least 83%, and 100%.

116. A mixture of deuterium-enriched compound of claim 111, wherein the abundance of deuterium in R₁ and R₂-R₃ is selected from at least 33%, at least 67%, and 100%.

117. A mixture of deuterium-enriched compound of claim 111, wherein the abundance of deuterium in R₁, R₆-R₈, and R₂₅-R₂₇ is selected from at least 14%, at least 29%, at least 43%, at least 57%, at least 71%, at least 86%, and 100%.

118. A mixture of deuterium-enriched compound of claim 111, wherein the abundance of deuterium in R₂-R₃, R₆-R₈, and R₂₅-R₂₇ is selected from at least 13%, at least 25%, at least 38%, at least 50%, at least 63%, at least 75%, at least 88%, and 100%.

119. A mixture of deuterium-enriched compound of claim 111, wherein the abundance of deuterium in R₁, R₂-R₃, R₆-R₈, and R₂₅-R₂₇ is selected from at least 11%, at least 22%, at least 33%, at least 44%, at least 56%, at least 67%, at least 78%, and 100%.

120. A mixture of deuterium-enriched compound of claim 111, wherein the abundance of deuterium in R₆-R₂₇ is selected from at least 5%, at least 9%, at least 14%, at least 18%, at least 23%, at least 27%, at least 32%, at least 36%, at least 41%, at least 45%, at least 50%, at least 55%, at least 59%, at least 64%, at least 68%, at least 73%, at least 77%, at least 82%, at least 86%, at least 91%, at least 95%, and 100%.

121. A mixture of deuterium-enriched compound of claim 111, wherein the abundance of deuterium in R₂-R₅ is selected from at least 25%, at least 50%, at least 75%, and 100%.

122. A mixture of deuterium-enriched compound of claim 111, wherein the abundance of deuterium in R₉-R₁₆ is selected from at least 13%, at least 25%, at least 38%, at least 50%, at least 63%, at least 75%, at least 88%, and 100%.

123. A mixture of deuterium-enriched compound of claim 111, wherein the abundance of deuterium in R₁₇-R₂₄ is selected from at least 13%, at least 25%, at least 38%, at least 50%, at least 63%, at least 75%, at least 88%, and 100%.

124. A mixture of deuterium-enriched compounds of claim 111, wherein the compounds are selected from the group consisting of:

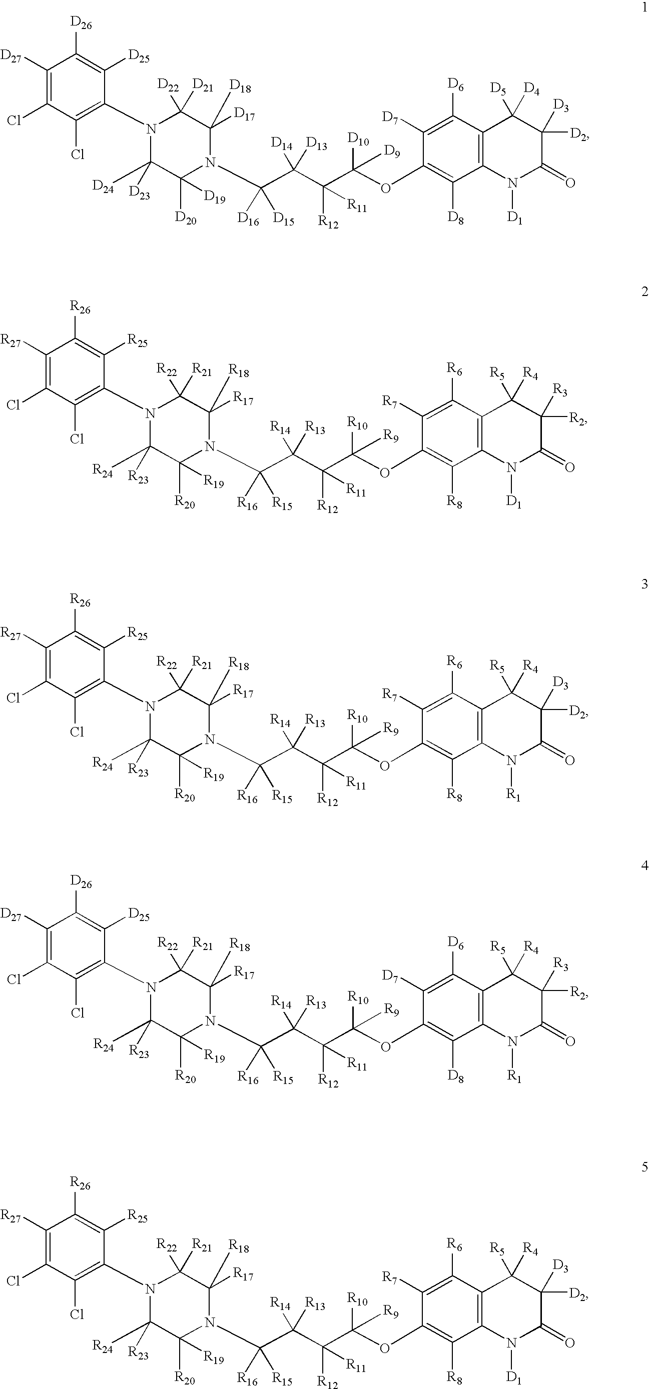

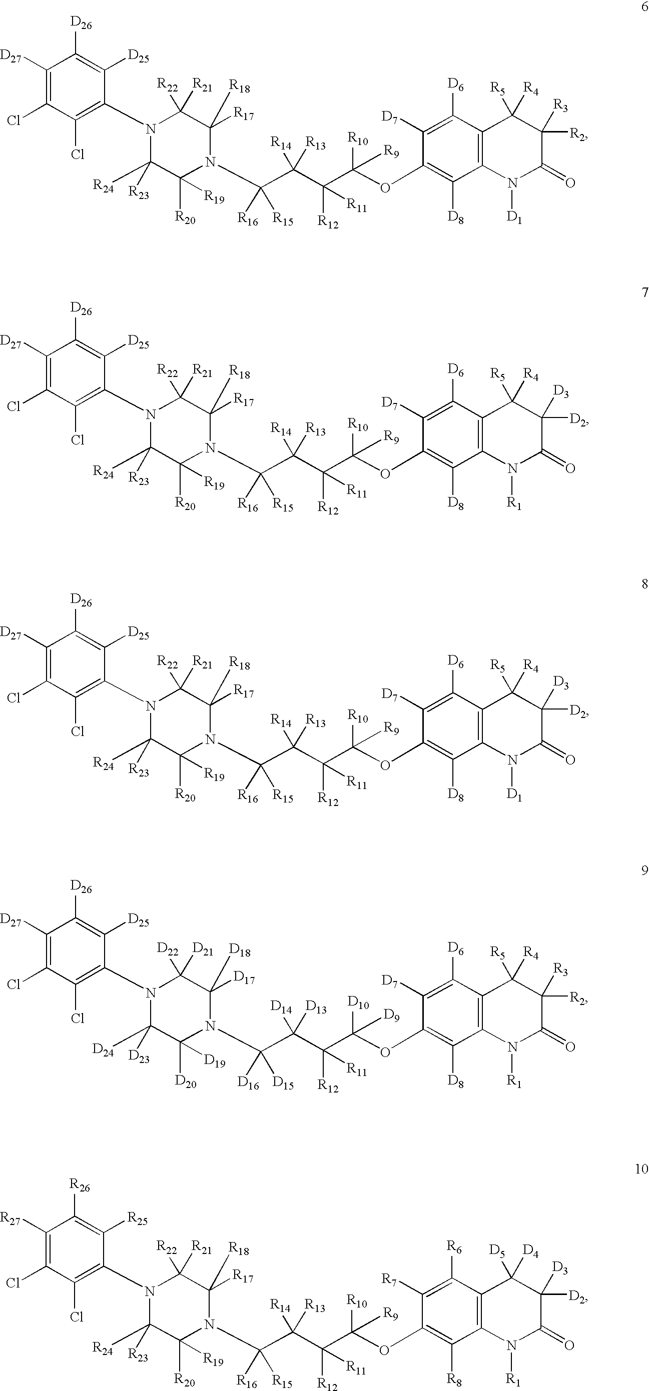

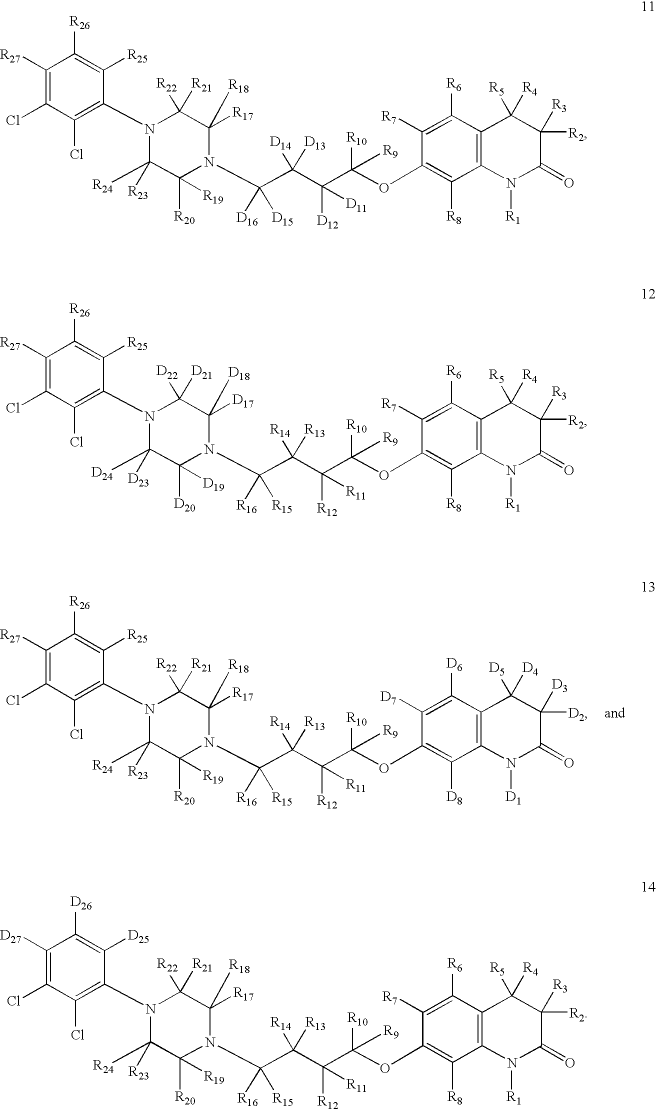

125. A mixture of deuterium-enriched compounds of claim 111, wherein the compounds are selected from the group consisting of:

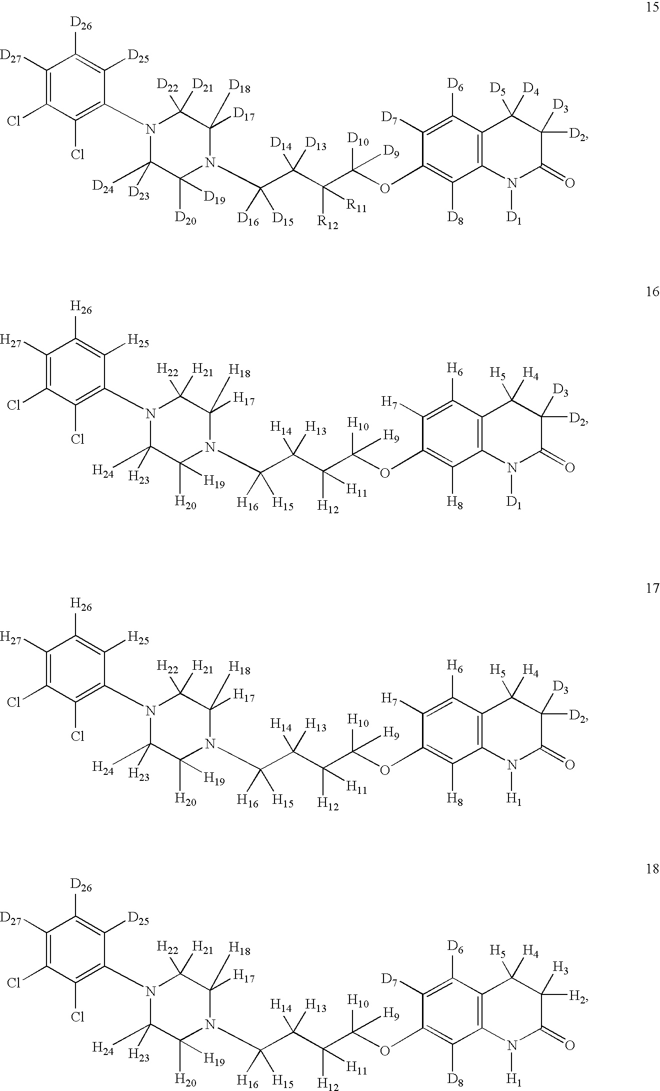

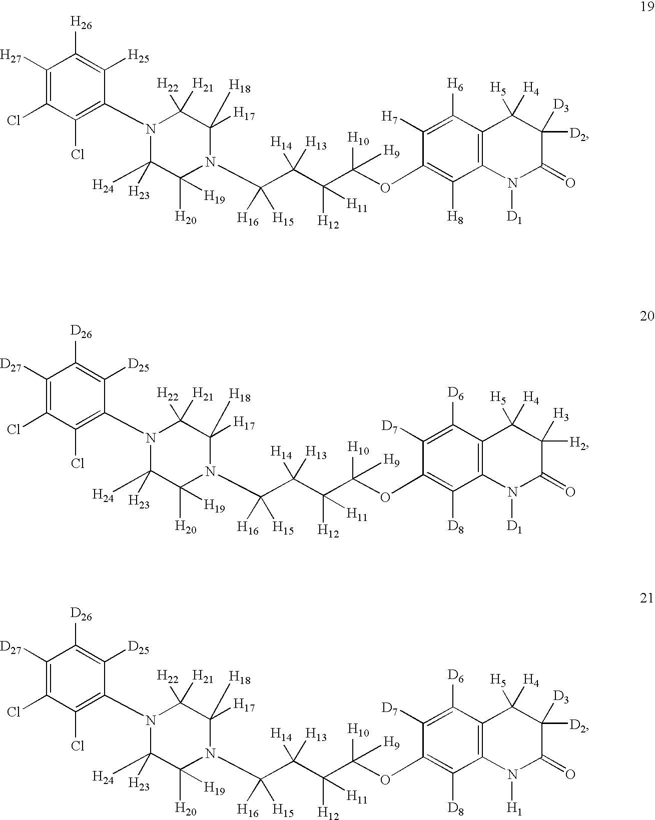

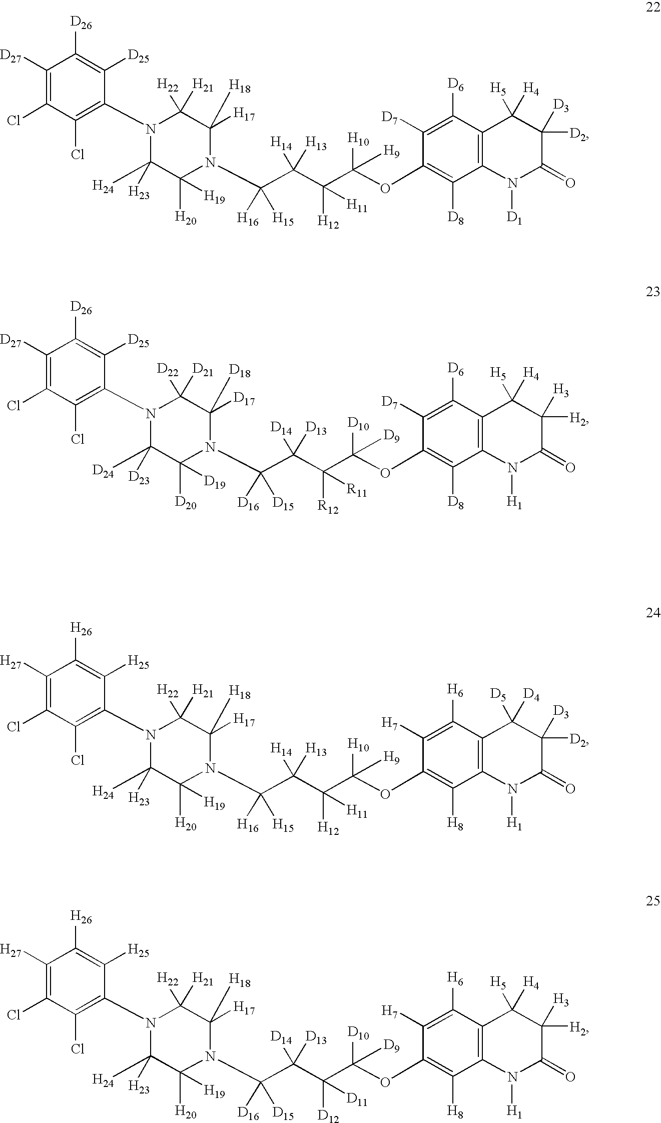

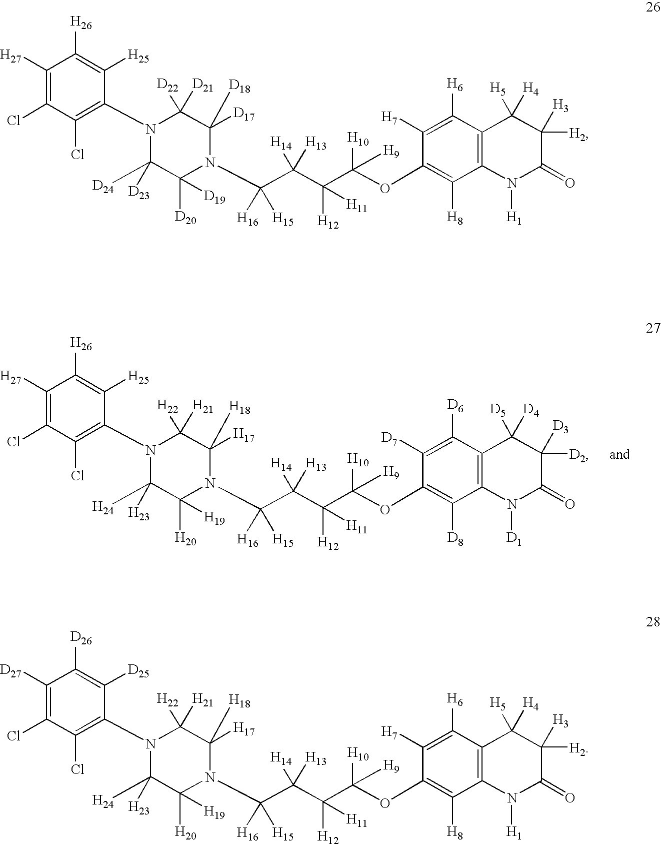

126. A pharmaceutical composition, comprising: a pharmaceutically acceptable carrier and a therapeutically effective amount of a compound of claim 81, or a pharmaceutically acceptable salt form thereof.

127. A method for treating schizophrenia comprising: administering, to a patient in need thereof, a therapeutically effective amount of a compound of claim 81, or a pharmaceutically acceptable salt form thereof.
